(19) United States
(12) Patent Application Publication
(10) Pub. No.: US 2025/0304550 A1
(43) Pub. Date: Oct. 2, 2025

(54) BROMODOMAIN AND EXTRA-TERMINAL (BET) SUBFAMILY BROMODOMAIN 1 (BD1) SELECTIVE INHIBITORS AND METHODS USING SAME

(71) Applicant: Baylor College of Medicine, Houston, TX (US)

(72) Inventors: Martin Matzuk, Houston, TX (US); Zhifeng Yu, Houston, TX (US); Ramkumar Modukuri, Houston, TX (US); Feng Li, Houston, TX (US); Damian Young, Houston, TX (US); Melek Nihan Ucisik, Houston, TX (US); John Faver, Houston, TX (US); Zhi Tan, Houston, TX (US); Zhuang Jin, Houston, TX (US); Justin Anglin, Houston, TX (US); Mingxing Teng, Houston, TX (US); Choel Kim, Houston, TX (US)

(21) Appl. No.: 18/854,888

(22) PCT Filed: Apr. 6, 2023
(86) PCT No.: PCT/US2023/017664

§ 371 Date: Oct. 7, 2024

Related U.S. Application Data
(60) Provisional application No. 63/328,325, filed on Apr. 7, 2022.

(51) Int. Cl. C07D 401/14; A61K 31/4545; A61K 31/4709; A61K 31/496; A61P 35/00; C07D 211/16; C07D 401/06

(52) U.S. Cl. CPC C07D 401/14; A61K 31/4545; A61K 31/4709; A61K 31/496; A61P 35/00; C07D 211/16; C07D 401/06

(57) ABSTRACT

The present disclosure relates to compounds which inhibit bromodomain testis (BRDT). In certain embodiments, the compound is a bromodomain and extra-terminal (BET) subfamily bromodomain 1 (BD1) selective compound. In certain embodiments, the compound selectively inhibits BD-1 over bromodomain 2 (BD2). The present disclosure further provides a method of inhibiting BRDT in a male subject, the method comprising administering to the male subject a therapeutically effective amount of a compound of the disclosure, whereby the male subject is provided a contraceptive effect. The present disclosure further provides a method of inhibiting BRD2, BRD3, BRD4, and/or BRDT in a subject with a cancer, inflammatory condition, infectious disease, and/or metabolic disorder in which one or more of BET proteins are regulators, the method comprising administering to the subject a therapeutically effective amount of a compound of the disclosure, thereby treating, preventing, and/or ameliorating the condition, disease, and/or disorder.

### CROSS-REFERENCE TO RELATED APPLICATIONS

This application claims priority under 35 U.S.C. § 119(e) to U.S. Provisional Patent Application No. 63/328,325, filed Apr. 7, 2022, which is incorporated herein by reference in its entirely.

### STATEMENT REGARDING FEDERALLY SPONSORED RESEARCH OR DEVELOPMENT

This invention was made with government support under Grant No. HD087157 awarded by the National Institutes of Health. The government has certain rights in the invention.

### BACKGROUND

BRDT (a testis-specific bromodomain-containing protein) is a member of the Bromodomain and Extra-Terminal (BET) family (along with BRD2, BRD3, and BRD4) and contains two tandem bromodomains. Bromodomain-containing proteins are implicated in cancer, inflammation, infectious disease, and metabolic disorders. BRDT is a validated germ cell target for nonhormonal contraceptive development. First generation pan-BET inhibitors such as JQ1 (tert-butyl (S)-2-(4-(4-chlorophenyl)-2,3,9-trimethyl-6H-thieno[3,2-f][1,2,4]triazolo[4,3-a][1,4]diazepin-6-yl)acetate) have been demonstrated as useful tool compounds in these studies, but the short half-life and rapid metabolism of JQ1 has motivated researchers to develop other pan-BET inhibitors.

Unfortunately, dose-limiting thrombocytopenia appears to be a common side effect among all pan-BET inhibitors, which may be due to poor selectivity among BET family members and between BD1 and BD2. Thus, the challenge of overcoming off-target tissue toxicity while maintaining anti-tumor efficacy remain for all pan-BET small molecule inhibitors.

Recent evidence indicates small molecule inhibition of the bromodomain 1 (BDT) of BRD4 has similar effects to inhibition of both bromodomains, while inhibition of BD2 may have more selective effects. In analogous fashion, it is the first bromodomain of BRDT (BRDT-BD1) that has been shown to be specifically essential for fertility in mice. Therefore BD1-specific BET inhibitors have been sought as both disease-fighting therapeutics and for eventual translation as non-hormonal male contraceptives.

Thus, there is a need in the art for selective BET-BD1 inhibitors and methods of use thereof. The present disclosure addresses this need.

### BRIEF SUMMARY OF THE INVENTION

In one aspect, the present disclosure provides certain compounds, including but not limited to compounds of formula (I), or a salt, solvate, prodrug, isotopologue, tautomer, or stereoisomer thereof:

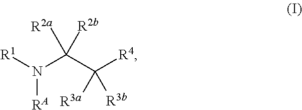

wherein the various substituents in the compound of formula (I) are defined elsewhere herein. The present disclosure further provides pharmaceutical compositions comprising the compounds of the present disclosure. In certain embodiments, the pharmaceutical compositions of the present disclosure comprise a pharmaceutically acceptable carrier.

In another aspect, the present disclosure provides a method of inhibiting bromodomain testis (BRDT) in a male subject. In certain embodiments, the method comprises administering to the subject a therapeutically effective amount of at least one compound of the present disclosure and/or a pharmaceutical composition of the present disclosure.

In another aspect, the present disclosure provides a method of promoting male contraception and/or infertility in a male subject. In certain embodiments, the method comprises administering to the subject a therapeutically effective amount of at least one compound of the present disclosure and/or a pharmaceutical composition of the present disclosure.

In another aspect, the present disclosure provides a method of minimizing and/or reducing spermatozoa number and/or motility in a male subject. In certain embodiments, the method comprises administering to the subject a therapeutically effective amount of at least one compound of the present disclosure and/or a pharmaceutical composition of the present disclosure.

In another aspect, the present disclosure provides a method of treating, preventing, and/or ameliorating cancer, an inflammatory condition, and/or a metabolic disorder in a subject. In certain embodiments, the method comprises administering to the subject a therapeutically effective amount of at least one compound of the present disclosure and/or a pharmaceutical composition of the present disclosure.

### BRIEF DESCRIPTION OF THE FIGURES

The drawings illustrate generally, by way of example, but not by way of limitation, various embodiments of the present application.

FIGS. 1A-1B show comparisons of normalized enrichment from parallel DNA-Encoded Chemistry Technology (DEC-Tec) screens against the isolated bromodomains of BRDT, wherein for each highlighted library member, the building blocks for cycles 1, 2, and 3 are shown from top to bottom. Selected library members are highlighted which show significant and selective enrichment for BRDT-BD1 compared to BRDT-BD2 (FIG. 1A), and binding of the library members to BRDT-BD1 shows competition with JQ1 (FIG. 1B).

FIG. 2 shows the structures and data relevant to the Mosher's amide analysis: Δ δ^{SR}(=δ_{S}−δ_{R}) data for the 4−3 (R,R) (R-MTPA amide) and 4-3 (R,S) (S-MTPA amide).

FIG. 3 shows activity of BD1-specific compounds from DNA-encoded chemical libraries. BRDT-BD1 and BRD4-BD1 inhibition of compounds 1, 2, and 3, compared to JQ1 as measured by AlphaScreen. Comparative potencies of each compound against the first and second bromodomains of BRDT and BRD4 are shown, with fold selectivity for BD1 versus BD2 provided immediately below. GraphPad Prism software was used to generate inhibition curves and to determine IC₅₀ values.

FIGS. 4A-4C show the selectivity of compounds of the present disclosure for BD1 and the BET family. FIG. 4A: BROMOscan bromodomain profiling of compound 4 on various bromodomains; phylogenetic tree of bromodomains demonstrating preferential binding of 4 for the BET subfamily BD1 domains using the BROMOscan bromodomain competition binding assay, wherein: I comprises CECR2, FALZ, GCN5L2, and PCAF; II comprises BRDT(1), BRD4(1), BRD3(1), BRD2(1), BRD2(2), BRD3(2), BRD4(2), and BRDT(2); III comprises CREBBP, EP300, BRWD3(2), PHIP(2), WDR9(2), BAZ1B, BRD8(1), and BRD8(2); IV comprises BRD1, BRPF3, BRPF1, BRD7, BRD9, BAZIA, ATAD2A, and ATAD2B; V comprises BAZ2A, BAZ2B, SP140, LOC93349, SP100, SP110, TRM33, TRIM24, and TRIM66; VI comprises MLL, and TRM28; VII comprises ASH1L, ZMYND11, TAF1L(2), FAF1(2), PRKCBP1, TAF1L(1), PHIP(1), WDR9(1), and BRWD3(1); and VIII comprises PB1(2), PB1(3), PB1(1), PB1(4), PB1(6), PB1(5), SMARCA4, and SMARCA2. FIG. 4B: BromoKdELECT dose-response curves which indicate that compounds 2 and 4 are strong binders of the BET subfamily BD1 domains, with the highest affinity for BRDT-BD1. FIG. 4C: BromoKdELECT summary table which indicates that compounds 2 and 4 are strong binders of the BET subfamily BD1 domains, with the highest affinity for BRDT-BD1.

FIGS. 5A-5E show detailed interactions between BRDT-BD1 and compound 4 (CDD-956). FIG. 5A: 2Fo-Fc map of compound 4 in complex with BRDT-BD1 contoured at 1σ. FIG. 5B: detailed interaction between BRDT-BD1 and compound 4; carbon atoms of compound 4 and an ordered water molecule that mediates the Y66-methylquinoline interaction are indicated; the ZA loop and αC helix are indicated; key interacting residues are shown in sticks. FIG. 5C: views of the compound 4 binding surface; the surface, except for side chain non-carbon atoms, oxygen atoms, nitrogen atoms, and sulfur atoms are indicated; FIG. 5D: the BRDT-BD2/CDD-1302 complex (PDB ID: 7L99) is superimposed with the BRDT-BD1/CDD-956 (compound 4) complex; the electrostatic potential surface belonging to the BRDT-BD1/CDD-956 (compound 4) complex is shown; the structures are aligned with an RMSD value of 0.56 Å between shared 100 CA atoms; both inhibitors occupy the KAc pocket but they access different adjacent grooves. FIG. 5E: the BRD4-BD1/iBET-BD1 complex (PDB ID: 6SWN) is superimposed with the BRDT-BD1/CDD-956 (compound 4) complex; they are aligned with an RMSD value of 0.95 Å between shared 95 CA atoms; both inhibitors occupy the KAc pocket, but only compound 4 extends significantly from this pocket.

FIGS. 6A-6B show four molecules capture in the BRDT-BD1/CDD-956 (compound 4) crystal. FIG. 6A: four BRDT-BD1 protein molecules are captured in the asymmetric unit with one molecule of compound 4 bound to each protein molecule. Bound compound 4 is shown in sticks. FIG. 6B: superimposition of four BRDT-BD1/CDD-956 (compound 4) complexes in the asymmetric unit; each of the four molecular units is nearly identical (showing RMSD values less than 0.5 Å between shared ˜100 CA atoms); each molecule is shown in cartoon with side chains of key compound 4 interacting residues shown in sticks.

FIG. 7 provides a LIGPLOT diagram showing interactions between BRDT-BD1 and compound 4.

FIGS. 8A-8E show that compounds 2 and 4 maintain BD1 selectivity, potency, and activity in cellular models; for all experiments JQ1 is used as a control. *=p<0.05, **=0.001<p<0.01, ***=0.0001<p<0.001, and ****=p<0.001. FIG. 8A: NanoBRET assays of compounds 2-5. FIG. 8B: Viability IC₅₀ of compounds of the present disclosure on 4 AML ccell lines after 72 hours of treatment by CellTiterGlo detection; results were normalized to DMSO-treated cells for the same length of time. FIG. 8C: Effect of compounds 2 and 4 on the cell cycle of MV4; 11 and MOLM-13 cells after 24 hours of treatment at the IC₅₀. FIG. 8D: Detection of Annexin V+ cells after 72 hours of treatment with compounds 2 and 4. FIG. 8E: Expression of MYC and GAPDH by RT-qPCR after 8 hours of treatment with compound 2 and 4.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g, the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “atm” as used herein refers to a pressure in atmospheres under standard conditions. Thus, 1 atm is a pressure of 101 kPa, 2 atm is a pressure of 202 kPa, and so on.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7.

Cycloalkyl groups further include polycyclic cycloalkyl groups such as, but not limited to, norbomyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonv)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl. 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanvl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3—, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “(+)-JQ1” or “JQ1” as used herein refers to

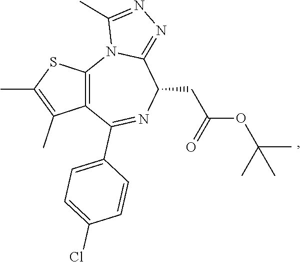 [SMILES: Cc1sc2c(c1C)C(c1ccc(Cl)cc1)=N[C@@H](CC(=O)OC(C)(C)C)c1nnc(C)n1-2]

also known as tert-butyl (S)-2-(4-(4-chlorophenyl)-2,3,9-trimethyl-6H-thieno[3,2-f][1,2,4]triazolo[4,3-a][1,4]diazepin-6-yl)acetate.

The term “monovalent” as used herein refers to a substituent connecting via a single bond to a substituted molecule. When a substituent is monovalent, such as, for example, F or C₁, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀)hydrocarbyl, wherein R can be hydrogen (in examples that include other carbon atoms) or a carbon-based moiety, and wherein the carbon-based moiety can be substituted or unsubstituted.

The term “room temperature” as used herein refers to a temperature of about 15° C. to 28° C.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

Certain abbreviations are used herein, including: DIEA, N,N-diisopropylethylamine; DECL, DEC-Tec, DNA-encoded chemistry technology; DNA-encoded compound library; DME, 1,2-dimethoxyethane; DMF, N,N-dimethylformamide; EtOAc, ethyl acetate; FMOC, fluorenylmethyloxycarbonyl, HATU, O-(7-azabenzotriazol-1-yl)-N,N,N′,N′-tetramethyluronium hexafluorophosphate; HLM, human liver microsomes; NaOH_{(aq)}, aqueous sodium hydroxide solution; MLM, mouse liver microsomes; Na₂CO₃₍ₛₐₜ₎, saturated aqueous sodium carbonate solution; Na₂SO₄, sodium sulfate; and tᵣ, retention time.

### DESCRIPTION

BD1 and BD2 share a high degree of sequence homology between BET family members. However, more significant structural differences exist between BD1 and BD2 themselves that can be exploited for selective ligand development to either domain. The compound RVX-208 was an early BD2-specific compound; however, it exhibited only moderate BD2 potency (>100 nM, IC₅₀=2 μM in a BRDT-BD2 AlphaScreen assay) and modest selectivity over BD1 domains (˜10 fold). More recent efforts have shown synthesis of pan-BD2 inhibitors with significantly enhanced potency and selectivity [e.g., GSK046 (Science 2020, 368:387-394), ABBV-744 (Nature 2020, 578:306-310), and others (Proc. Natl. Acad. Sci. USA 2021, 118)].

To identify novel BRDT-BD1 inhibitors, a screening campaign was initiated, as described herein, using DNA-encoded chemical libraries (DECLs), which are becoming an established lead generation platform for various therapeutic targets.

Given that BRDT-BD1 is necessary for fertility in mice and for contraceptive effects in the testes, a parallel screen of BRDT-BD1 and BRDT-BD2 were performed with the DNA-encoded chemical library. Herein is described the discovery of a DEC-Tec inhibitor series that is both potent and highly selective for BD1 of BET subfamily members versus BD2.

Confirmed by BROMOscan, subnanomolar K_{d} values were determined for all BET BD1 domains, and >5000-fold selectivity was identified over BET BD2 domains by BromoKdELECT for BRD3, BRD4, and BRDT, while BRD2 shows 42-fold BD1 selectivity versus BD2. Molecular docking studies confirmed that the stereochemistry of the compounds drives the selectivity of BD1 versus BD2. BD1-specificity was maintained in cell lines and marked anti-leukemic activity was seen. In summary, this work reinforces the importance of chirality to achieve BD1 selectivity, and these new, highly potent BD1 inhibitors are useful tools both in the study of spermatogenesis and for translational studies in other disease models.

### Compounds

In one aspect, the present disclosure provides a compound of formula (I), or a salt, solvate, prodrug, isotopologue, tautomer, or stereoisomer thereof:

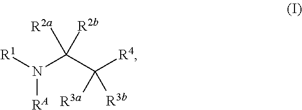

wherein:

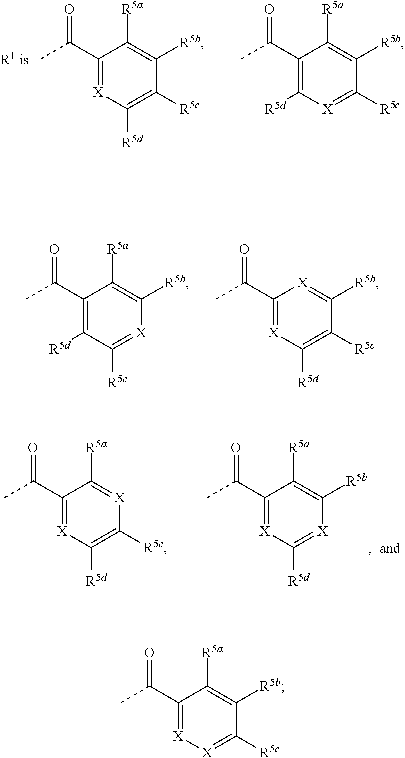

R^{2a} and R^{2b} are each independently selected from the group consisting of H, optionally substituted C₁-C₆ alkyl, and C(═O)N(R^{a})(R^{b}),
   wherein at least one of R^{2a} and R^{2b} is H;
R^{3a} and R^{3b} are each independently selected from the group consisting of H and C₁-C₆ alkyl;
R⁴ is

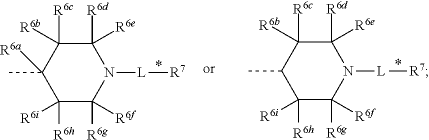

R^{5a}, R^{5b}, R^{5c}, R^{5d}, and R^{5e}, if present, are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂OR^{a}, S(═O)₂R^{a}, S(═O)₂N(R^{a})(R^{b}), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
   wherein two vicinal substituents selected from the group consisting of R^{5a}, R^{5b}, R^{5c}, R^{5d}, and R₅ₑ, if present, may combine to form an optionally substituted phenyl or optionally substituted C₂-C₈ heteroaryl;
R^{6a}, R^{6b}, R^{6c}, R^{6d}, R^{6e}, R^{6f}, R^{6g}, R^{6h}, and R⁶ⁱ, if present, are each independently selected from the group consisting of H, halogen, optionally substituted C₁-C₆ alkyl, C₁-C₆ alkoxy, C₁-C₆ haloalkyl, C₁-C₆ haloalkoxy, optionally substituted C₃-C₈ cycloalkyl, and optionally substituted C₂-C₈ heterocyclyl;
R⁷ is

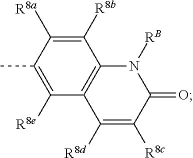

R^{8a}, R^{8b}, R^{8c}, R^{8d}, and R^{8e} are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂OR^{a}, S(═O)₂R^{a}, S(═O)₂N(R^{a})(R^{b}), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
X is selected from the group consisting of N and C(R^{5e});
L is selected from the group consisting of —C(═O)(optionally substituted C₁-C₃ alkylene)-*, —(C═O)—*, and -(optionally substituted C₁-C₃ alkylene)C(═O)—*,
   wherein * indicates the bond between L and R⁷;
R^{A} and R^{B} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl; and
R^{a} and R^{b} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl.

In certain embodiments, the compound of formula (I), is

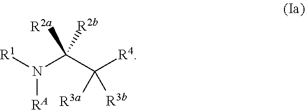

In certain embodiments, the compound of formula (I) is

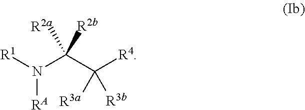

In certain embodiments, X is N. In certain embodiments, X is CH.

In certain embodiments, R^{5a} is H. In certain embodiments, R^{5a} is D. In certain embodiments, R^{5a} is methyl. In certain embodiments, R^{5a} is ethyl. In certain embodiments, R^{5a} is CF₃. In certain embodiments, R^{5a} is phenyl. In certain embodiments, R^{5a} is phenyl substituted with at least one substituent selected from the group consisting of C₁-C₆ alkyl and halogen. In certain embodiments, R^{5a} is 2,4-dimethylphenyl. In certain embodiments, R^{5a} is 4-chlorophenyl. In certain embodiments, R^{5a} is 3-fluoro-4-chlorophenyl.

In certain embodiments, R^{5b} is H. In certain embodiments, R^{5b} is D. In certain embodiments, R^{5b} is methyl. In certain embodiments, R^{5b} is ethyl. In certain embodiments, R^{5b} is CF₃. In certain embodiments, R^{5b} is phenyl. In certain embodiments, R^{5b} is phenyl substituted with at least one substituent selected from the group consisting of C₁-C₆ alkyl and halogen. In certain embodiments, R^{5b} is 2,4-dimethylphenyl. In certain embodiments, R^{5b} is 4-chlorophenyl. In certain embodiments, R^{5b} is 3-fluoro-4-chlorophenyl.

In certain embodiments, Rio is H. In certain embodiments, R^{5c} is D. In certain embodiments, R^{5c} is methyl. In certain embodiments, R^{5c} is ethyl. In certain embodiments, Rio is CF₃. In certain embodiments, R^{5c} is phenyl. In certain embodiments, R^{5c} is phenyl substituted with at least one substituent selected from the group consisting of C₁-C₆ alkyl and halogen. In certain embodiments, Rio is 2,4-dimethylphenyl. In certain embodiments, R^{5c} is 4-chlorophenyl. In certain embodiments, R^{5c}, is 3-fluoro-4-chlorophenyl.

In certain embodiments, R^{5d} is H. In certain embodiments, R^{5d} is D. In certain embodiments, R^{5d} is methyl. In certain embodiments, R^{5d} is ethyl. In certain embodiments, R^{5d} is CF₃. In certain embodiments, R^{5d} is phenyl. In certain embodiments, R^{5d} is phenyl substituted with at least one substituent selected from the group consisting of C₁-C₆ alkyl and halogen. In certain embodiments, R^{5d} is 2,4-dimethylphenyl. In certain embodiments, R^{5d} is 4-chlorophenyl. In certain embodiments, R^{5d} is 3-fluoro-4-chlorophenyl.

In certain embodiments, R^{5e} is H. In certain embodiments, R^{5e} is D. In certain embodiments, R^{5e} is methyl. In certain embodiments, R^{5e} is ethyl. In certain embodiments, R^{5e} is CF₃. In certain embodiments, R^{5e} is phenyl. In certain embodiments, R^{5e} is phenyl substituted with at least one substituent selected from the group consisting of C₁-C₆ alkyl and halogen. In certain embodiments, R^{5e} is 2,4-dimethylphenyl. In certain embodiments, R^{5e} is 4-chlorophenyl. In certain embodiments, R⁵ is 3-fluoro-4-chlorophenyl.

In certain embodiments, R¹ is

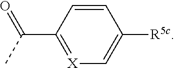

In certain embodiments, R¹ is

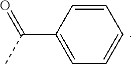

In certain embodiments, R¹ is

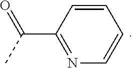

In certain embodiments, R¹ is

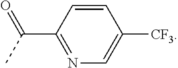

In certain embodiments, R¹ is

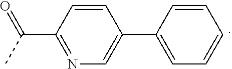

In certain embodiments, R¹ is

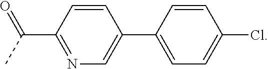

In certain embodiments, R¹ is

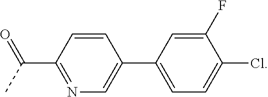

In certain embodiments, R¹ is

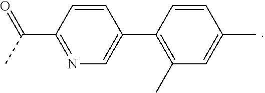

In certain embodiments, R¹ is

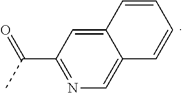

In certain embodiments, R¹ is

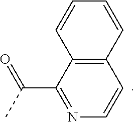

In certain embodiments, R^{2a} is H. In certain embodiments, R^{2a} is D. In certain embodiments, R^{2a} is methyl. In certain embodiments, R^{2a} is ethyl.

In certain embodiments, R^{2b} is H. In certain embodiments, R^{2b} is D. In certain embodiments, R^{2b} is methyl. In certain embodiments, R^{2b} is ethyl.

In certain embodiments, R^{2a} is H and R^{2b} is H. In certain embodiments, R^{2a} is C(═O)NHMe and R^{2b} is H. In certain embodiments, R^{2a} is H and R^{2b} is C(═O)NHMe. In certain embodiments, R^{2a} is Me and R^{2b} is H. In certain embodiments, R^{2a} is H and R^{2b} is Me.

In certain embodiments, R^{3a} is H. In certain embodiments, R^{3a} is D. In certain embodiments, R^{3a} is methyl. In certain embodiments, R^{3a} is ethyl.

In certain embodiments, R^{3b} is H. In certain embodiments, R^{3b} is D. In certain embodiments, R^{3b} is methyl. In certain embodiments, R^{3b} is ethyl.

In certain embodiments, R^{6a} is H. In certain embodiments, R^{6a} is D. In certain embodiments, R^{6a} is methyl. In certain embodiments, R^{6a} is ethyl.

In certain embodiments, R^{6b} is H. In certain embodiments, R^{6b} is D. In certain embodiments, R^{6b} is methyl. In certain embodiments, R^{6b} is ethyl.

In certain embodiments, R^{6c} is H. In certain embodiments, R^{6c} is D. In certain embodiments, R^{6c} is methyl. In certain embodiments, R^{6c} is ethyl.

In certain embodiments, R^{6d} is H. In certain embodiments, R^{6d} is D. In certain embodiments, R^{6d} is methyl. In certain embodiments, R^{6d} is ethyl.

In certain embodiments, R^{6e} is H. In certain embodiments, R^{6e} is D. In certain embodiments, R^{6e} is methyl. In certain embodiments, R^{6e} is ethyl.

In certain embodiments, R^{6f} is H. In certain embodiments. R^{6f} is D. In certain embodiments, R^{6f} is methyl. In certain embodiments, R^{6f} is ethyl.

In certain embodiments, R^{6g} is H. In certain embodiments, R^{6g} is D. In certain embodiments, R^{6g} is methyl. In certain embodiments, R^{6g} is ethyl.

In certain embodiments, R^{6h} is H. In certain embodiments, R^{6h} is D. In certain embodiments, R^{6h} is methyl. In certain embodiments, R^{6h} is ethyl.

In certain embodiments, R⁶ⁱ is H. In certain embodiments, R⁶ⁱ is D. In certain embodiments, R⁶ⁱ is methyl. In certain embodiments, R⁶ⁱ is ethyl.

In certain embodiments, at least one of R^{6a}, R^{6b}, R^{6c}, R^{6d}, R^{6e}, R^{6f}, R^{6g}, R^{6h} and R⁶ⁱ is H. In certain embodiments, at least two of R^{6a}, R^{6b}, R^{6c}, R^{6d}, R^{6e}, R^{6f}, R^{6g}, R^{6h}, and R⁶ⁱ are H.

In certain embodiments, at least three of R^{6a}, R^{6b}, R^{6c}, R^{6d}, R^{6e}, R^{6f}, R^{6g}, R^{6h}, and R⁶ⁱ are H. In certain embodiments, at least four of R^{6a}, R^{6b}, R^{6c}, R^{6d}, R^{6e}, R^{6f}, R^{6g}, R^{6h}, and R⁶ⁱ are H. In certain embodiments, at least five of R^{6a}, R^{6b}, R^{6c}, R^{6d}, R^{6e}, R^{6f}, R^{6g}, R^{6h}, and R⁶ⁱ are H. In certain embodiments, at least six of R^{6a}, R^{6b}, R^{6c}, R^{6d}, R^{6e}, R^{6f}, R^{6g}, R^{6h}, and R⁶ⁱ are H. In certain embodiments, at least seven of R^{6a}, R^{6b}, R^{6c}, R^{6d}, R^{6c}, R^{6f}, R^{6g}, R^{6h}, and R⁶ⁱ are H. In certain embodiments, at least eight of R^{6a}, R^{6b}, R^{6c}, R^{6d}, R^{6e}, R^{6f}, R^{6g}, R^{6h}, and R⁶ⁱ are H. In certain embodiments, each of R^{6a}, R^{6b}, R^{6c}, R^{6d}, R^{6e}, R^{6f}, R^{6g}, R^{6h}, and R⁶ⁱ is H.

In certain embodiments, L is —C(═O)CH₂—*.

In certain embodiments, R^{8a} is H. In certain embodiments, R^{8a} is D. In certain embodiments, R^{8a} is methyl. In certain embodiments, R^{8a} is ethyl.

In certain embodiments, R^{8b} is H. In certain embodiments, R^{8b} is D. In certain embodiments, R^{8b} is methyl. In certain embodiments, R^{8b} is ethyl.

In certain embodiments, R^{8c} is H. In certain embodiments, R^{8c} is D. In certain embodiments, R^{8c} is methyl. In certain embodiments, R^{8c} is ethyl.

In certain embodiments, R^{8d} is H. In certain embodiments, R^{8d} is D. In certain embodiments, R^{8d} is methyl. In certain embodiments, R^{8d} is ethyl.

In certain embodiments, R^{8e} is H. In certain embodiments, R^{8e} is D. In certain embodiments, R^{8e} is methyl. In certain embodiments, R^{8e} is ethyl.

In certain embodiments, R⁷ is

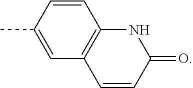

In certain embodiments, R⁷ is

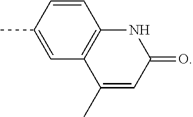

In certain embodiments, R⁴ is

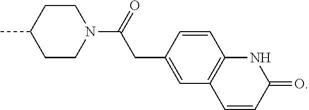

In certain embodiments, R⁴ is

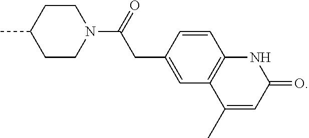

In certain embodiments, R⁴ is

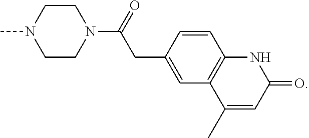

In certain embodiments, each occurrence of alkyl, haloalkyl, alkoxy, haloalkoxy, cycloalkyl, heterocyclyl, benzyl, phenyl, naphthyl, and heteroaryl is independently optionally substituted with at least one substituent selected from the group consisting of C₁-C₆ alkyl, C₃-C₈ cycloalkyl, C₃-C₆ allyl, C₃-C₆ propargyl, C₁-C₆ hydroxyalkyl, halogen, NO₂, CN, OH, NH₂, NH(C₁-C₆ alkyl), N(C₁-C₆ alkyl)₂, NH(C₆-C₁₀ aryl), N(C₆-C₁₀ aryl)₂, C₁-C₆ alkoxy, C₃-C₈ cycloalkoxy, C₁-C₃ haloalkyl, C₁-C₆ haloalkoxy, C₃-C₈ halocycloalkoxy, benzyl, phenyl, naphthyl, C₂-C₈ heterocyclyl, C(═O)H, C(═O)(C₁-C₆ alkyl), C(═O)(C₆-C₁₀ aryl), C(═O)O(benzyl), C(═O)(C₃-C₈ cycloalkyl), C(═O)OH, C(═O)O(C₁-C₆ alkyl), C(═O)O(C₆-C₁₀ aryl), OC(═O)H, OC(═O)(C₁-C₆ alkyl), OC(═O)(C₆-C₁₀ aryl), OC(═O)OH, OC(═O)O(C₁-C₆ alkyl), OC(═O)O(C₆-C₁₀ aryl), SH, S(C₁-C₆ alkyl), S(C₆-C₁₀ aryl), S(═O)(C₁-C₆ alkyl), S(═O)(C₆-C₁₀ aryl), S(═O)₂₀H, S(═O)₂₀(C₁-C₆ alkyl), S(═O)₂₀(C₆-Cia aryl), S(═O)₂(C₁-C₆ alkyl), S(═O)₂(C₆-C₁₀ aryl), S(═O)₂NH₂, S(═O)₂NH(C₁-C₆ alkyl), S(═O)₂N(C₁-C₆ alkyl)₂, S(═O)₂NH(C₆-C₁₀ aryl), S(═O)₂N(C₆-C₁₀ aryl)₂, S(═O)₂NHC(═O)NH₂, S(═O)₂N(C₁-C₆ alkyl)C(═O)NH₂, S(═O)₂NHC(═O)NH(C₁-C₆ alkyl), S(═O)₂N(C₁-C₆ alkyl)C(═O)NH(C₁-C₆ alkyl), S(═O)₂NHC(═O)NH(C₆-C₁₀ aryl), NHS(═O)₂(C₁-C₆ alkyl), N(C₁-C₆ alkyl)S(═O)₂(C₁-C₆ alkyl), NHS(═O)₂(C₆-C₁₀ aryl), N(C₁-C₆ alkyl)S(═O)₂(C₆-C₁₀ aryl), NHC(═O)H, NHC(═O)(C₁-C₆ alkyl), N(C₁-C₆ alkyl)C(═O)(C₁-C₆ alkyl), NHC(═O)(C₆-C₁₀ aryl), N(C₁-C₆ alkyl)C(═O)(C₆-C₁₀ aryl), C(═O)NH₂, C(═O)NH(C₁-C₆ alkyl), C(═O)N(C₁-C₆ alkyl)₂, C(═O)NH(C₆-C₁₀ aryl), C(═O)N(C₁-C₆ alkyl)(C₆-C₁₀ aryl), and C(═O)N(C₆-C₁₀ aryl)₂.

In certain embodiments, each optional substituent in the alkyl, haloalkyl, alkoxy, haloalkoxy, cycloalkyl, heterocyclyl, benzyl, phenyl, naphthyl, and heteroaryl is further optionally substituted with at least one substituent selected from the group consisting of C₁-C₆ alkyl, C₁-C₃ haloalkyl, C₁-C₃ haloalkoxy, C₁-C₆ heteroalkyl, halogen, CN, NO₂, OH, O(C₁-C₆ alkyl), NH₂, NH(C₁-C₆ alkyl), N(C₁-C₆ alkyl)₂, C(═O)OH, C(═O)O(C₁-C₆ alkyl), C(═O)NH₂, C(═O)NH(C₁-C₆ alkyl), C(═O)N(C₁-C₆ alkyl)₂, NH(C═NH)NH₂, and imidazolyl.

In certain embodiments, the compound is selected from the group consisting of.
5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(R)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(S)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
N-(1-(methylamino)-1-oxo-3-(1-(2-(2-oxo-1,2-dihydroquinolin-6-yl)acetyl)piperidin-4-yl)propan-2-yl)-5-phenylpicolinamide;
(R)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
(S)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(R)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(S)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
5-(2,4-dimethylphenyl)-N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)benzamide;
N-(2-(1-(2-(4-methyl-2-oxo-1,2-dihydroquinolin-6-yl)acetyl)piperidin-4-yl)ethyl)quinoline-2-carboxamide;
N-(2-(1-(2-(4-methyl-2-oxo-1,2-dihydroquinolin-6-yl)acetyl)piperidin-4-yl)ethyl)isoquinoline-3-carboxamide;
N-(2-(1-(2-(4-methyl-2-oxo-1,2-dihydroquinolin-6-yl)acetyl)piperidin-4-yl)ethyl)isoquinoline-1-carboxamide; and
N-(2-(4-(2-(4-methyl-2-oxo-1,2-dihydroquinolin-6-yl)acetyl)piperazin-1-yl)ethyl)picolinamide.

TABLE 1

| Cmpd No. | Compound |
|---|---|
| 1 (CDD- 724) | 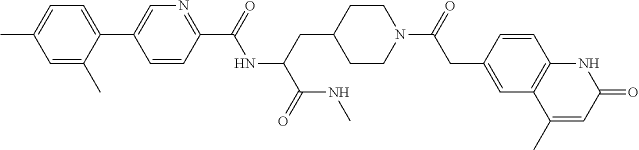 [SMILES: CNC(=O)C(CC1CCN(C(=O)Cc2ccc3[nH]c(=O)cc(C)c3c2)CC1)NC(=O)c1ccc(-c2ccc(C)cc2C)cn1] 5-(2,4-dimethylphenyl)-N-(3-(1-(2-(4-methyl-2-oxo-1,2-dihydroquinolin-6- yl) acetyl) piperidin-4-yl)-1-(methylamino)-1-oxopropan-2-yl)picolinamide |
| 2 (CDD- 787) | 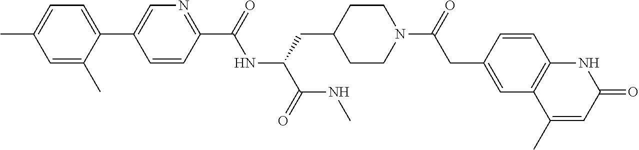 [SMILES: CNC(=O)[C@@H](CC1CCN(C(=O)Cc2ccc3[nH]c(=O)cc(C)c3c2)CC1)NC(=O)c1ccc(-c2ccc(C)cc2C)cn1] (R)-5-(2,4-dimethylphenyl)-N-(3-(1-(2-(4-methyl-2-oxo-1,2-dihydroquinolin- 6-yl) acetyl) piperidin-4-yl)-1-(methylamino)-1-oxopropan-2-yl)picolinamide |
| 3 (CDD- 786) | 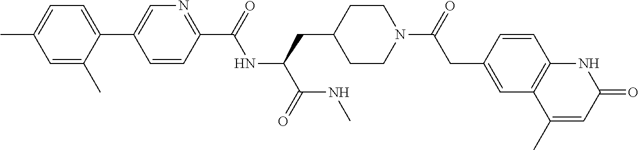 [SMILES: CNC(=O)[C@H](CC1CCN(C(=O)Cc2ccc3[nH]c(=O)cc(C)c3c2)CC1)NC(=O)c1ccc(-c2ccc(C)cc2C)cn1] (S)-5-(2,4-dimethylphenyl)-N-(3-(1-(2-(4-methyl-2-oxo-1,2-dihydroquinolin- 6-yl) acetyl) piperidin-4-yl)-1-(methylamino)-1-oxopropan-2-yl)picolinamide |
| 4 (CDD- 956) | 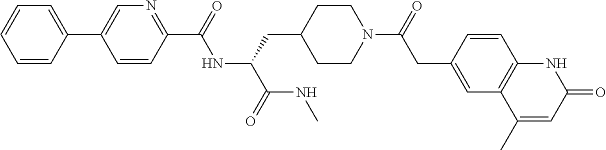 [SMILES: CNC(=O)[C@@H](CC1CCN(C(=O)Cc2ccc3[nH]c(=O)cc(C)c3c2)CC1)NC(=O)c1ccc(-c2ccccc2)cn1] (R)-N-(3-(1-(2-(4-methyl-2-oxo-1,2-dihydroquinolin-6-yl)acetyl)piperidin-4- yl)-1-(methylamino)-1-oxopropan-2-yl)-5-phenylpicolinamide |
| 5 (CDD- 2107) | 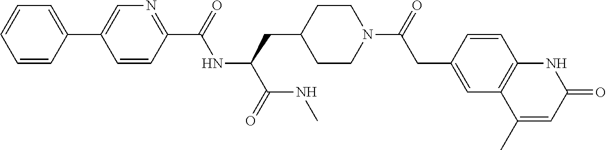 [SMILES: CNC(=O)[C@H](CC1CCN(C(=O)Cc2ccc3[nH]c(=O)cc(C)c3c2)CC1)NC(=O)c1ccc(-c2ccccc2)cn1] (S)-N-(3-(1-(2-(4-methyl-2-oxo-1,2-dihydroquinolin-6-yl)acetyl)piperidin-4- yl)-1-(methylamino)-1-oxopropan-2-yl)-5-phenylpicolinamide |
| 6 (CDD- 818) | 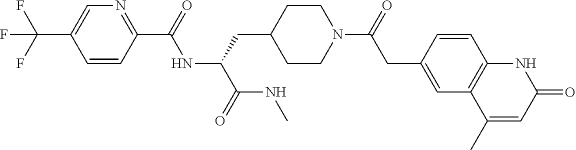 [SMILES: CNC(=O)[C@@H](CC1CCN(C(=O)Cc2ccc3[nH]c(=O)cc(C)c3c2)CC1)NC(=O)c1ccc(C(F)(F)F)cn1] (R)-N-(3-(1-(2-(4-methyl-2-oxo-1,2-dihydroquinolin-6-yl)acetyl)piperidin-4- yl)-1-(methylamino)-1-oxopropan-2-yl)-5-(trifluoromethyl)picolinamide |
| 7 (CDD- 819) | 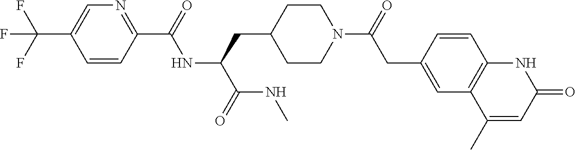 [SMILES: CNC(=O)[C@H](CC1CCN(C(=O)Cc2ccc3[nH]c(=O)cc(C)c3c2)CC1)NC(=O)c1ccc(C(F)(F)F)cn1] (S)-N-(3-(1-(2-(4-methyl-2-oxo-1,2-dihydroquinolin-6-yl)acetyl)piperidin-4- yl)-1-(methylamino)-1-oxopropan-2-yl)-5-(trifluoromethyl)picolinamide |
| 8 (CDD- 981) | 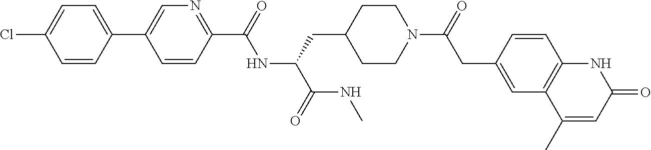 [SMILES: CNC(=O)[C@@H](CC1CCN(C(=O)Cc2ccc3[nH]c(=O)cc(C)c3c2)CC1)NC(=O)c1ccc(-c2ccc(Cl)cc2)cn1] (R)-5-(4-chlorophenyl)-N-(3-(1-(2-(4-methyl-2-oxo-1,2-dihydroquinolin-6- yl)acetyl)piperidin-4-yl)-1-(methylamino)-1-oxopropan-2-yl)picolinamide |
| 9 (CDD- 982) | 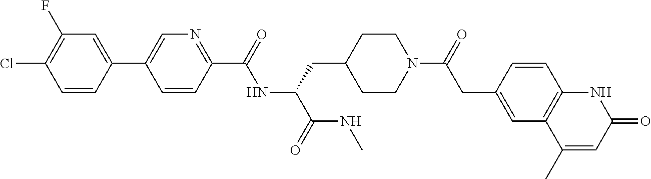 [SMILES: CNC(=O)[C@@H](CC1CCN(C(=O)Cc2ccc3[nH]c(=O)cc(C)c3c2)CC1)NC(=O)c1ccc(-c2ccc(Cl)c(F)c2)cn1] (R)-5-(4-chloro-3-fluorophenyl)-N-(3-(1-(2-(4-methyl-2-oxo-1,2- dihydroquinolin-6-yl)acetyl)piperidin-4-yl)-1-(methylamino)-1-oxopropan-2- yl)picolinamide |
| 10 (CDD- 986) | 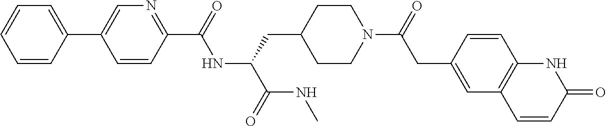 [SMILES: CNC(=O)[C@@H](CC1CCN(C(=O)Cc2ccc3[nH]c(=O)ccc3c2)CC1)NC(=O)c1ccc(-c2ccccc2)cn1] (R)-N-(1-(methylamino)-1-oxo-3-(1-(2-(2-oxo-1,2-dihydroquinolin-6- yl)acetyl)piperidin-4-yl)propan-2-yl)-5-phenylpicolinamide |
| 11 (CDD- 784) | 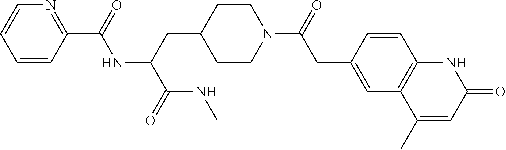 [SMILES: CNC(=O)C(CC1CCN(C(=O)Cc2ccc3[nH]c(=O)cc(C)c3c2)CC1)NC(=O)c1ccccn1] N-(3-(1-(2-(4-methyl-2-oxo-1,2-dihydroquinolin-6-yl)acetyl)piperidin-4-yl)- 1-(methylamino)-1-oxopropan-2-yl)picolinamide |
| 12 (CDD- 785) | 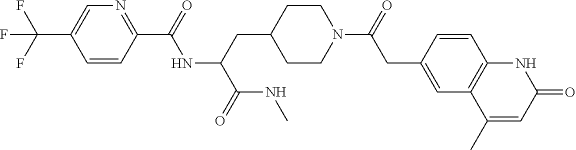 [SMILES: CNC(=O)C(CC1CCN(C(=O)Cc2ccc3[nH]c(=O)cc(C)c3c2)CC1)NC(=O)c1ccc(C(F)(F)F)cn1] N-(3-(1-(2-(4-methyl-2-oxo-1,2-dihydroquinolin-6-yl)acetyl)piperidin-4-yl)- 1-(methylamino)-1-oxopropan-2-yl)-5-(trifluoromethyl)picolinamide |
| 13 (CDD- 1146) | 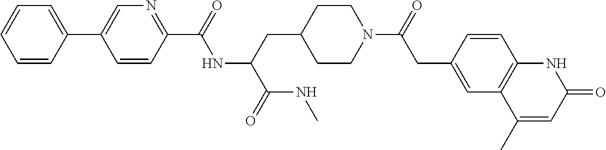 [SMILES: CNC(=O)C(CC1CCN(C(=O)Cc2ccc3[nH]c(=O)cc(C)c3c2)CC1)NC(=O)c1ccc(-c2ccccc2)cn1] N-(3-(1-(2-(4-methyl-2-oxo-1,2-dihydroquinolin-6-yl)acetyl)piperidin-4-yl)- 1-(methylamino)-1-oxopropan-2-yl)-5-phenylpicolinamide |
| 14 (CDD- 906) | 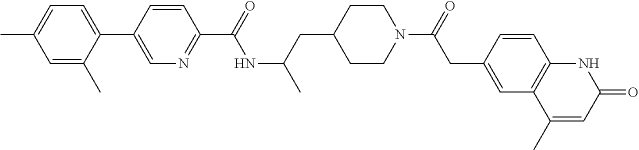 [SMILES: Cc1ccc(-c2ccc(C(=O)NC(C)CC3CCN(C(=O)Cc4ccc5[nH]c(=O)cc(C)c5c4)CC3)nc2)c(C)c1] 5-(2,4-dimethylphenyl)-N-(1-(1-(2-(4-methyl-2-oxo-1,2-dihydroquinolin-6- yl)acetyl)piperidin-4-yl)propan-2-yl)picolinamide |
| 15 (CDD- 813) | 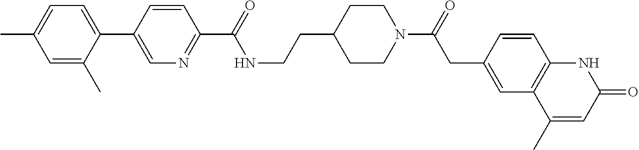 [SMILES: Cc1ccc(-c2ccc(C(=O)NCCC3CCN(C(=O)Cc4ccc5[nH]c(=O)cc(C)c5c4)CC3)nc2)c(C)c1] 5-(2,4-dimethylphenyl)-N-(2-(1-(2-(4-methyl-2-oxo-1,2-dihydroquinolin-6- yl)acetyl)piperidin-4-yl)ethyl)picolinamide |
| 16 (CDD- 767) | 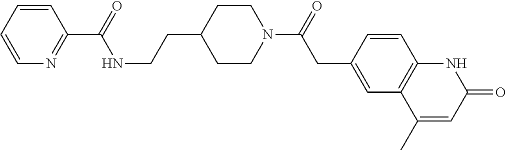 [SMILES: Cc1cc(=O)[nH]c2ccc(CC(=O)N3CCC(CCNC(=O)c4ccccn4)CC3)cc12] N-(2-(1-(2-(4-methyl-2-oxo-1,2-dihydroquinolin-6-yl)acetyl)piperidin-4- yl)ethyl)picolinamide |
| 17 (CDD- 779) | 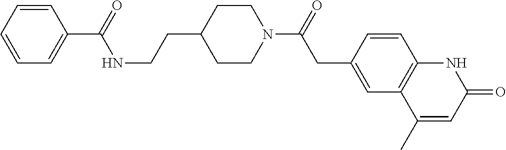 [SMILES: Cc1cc(=O)[nH]c2ccc(CC(=O)N3CCC(CCNC(=O)c4ccccc4)CC3)cc12] N-(2-(1-(2-(4-methyl-2-oxo-1,2-dihydroquinolin-6-yl)acetyl)piperidin-4- yl)ethyl)benzamide |
| 18 (CDD- 855) | 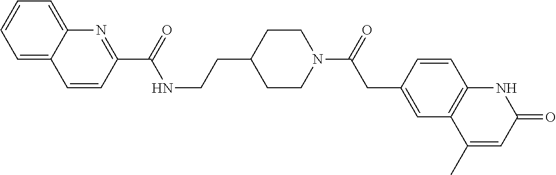 [SMILES: Cc1cc(=O)[nH]c2ccc(CC(=O)N3CCC(CCNC(=O)c4ccc5ccccc5n4)CC3)cc12] N-(2-(1-(2-(4-methyl-2-oxo-1,2-dihydroquinolin-6-yl)acetyl)piperidin-4- yl)ethyl)quinoline-2-carboxamide |
| 19 (CDD- 854) | 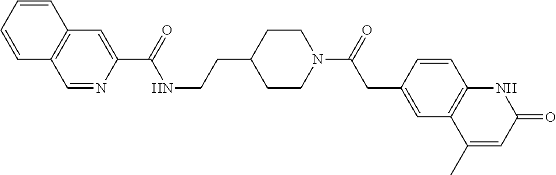 [SMILES: Cc1cc(=O)[nH]c2ccc(CC(=O)N3CCC(CCNC(=O)c4cc5ccccc5cn4)CC3)cc12] N-(2-(1-(2-(4-methyl-2-oxo-1,2-dihydroquinolin-6-yl)acetyl)piperidin-4- yl)ethyl)isoquinoline-3-carboxamide |
| 20 (CDD- 853) | 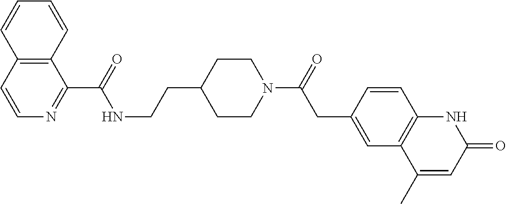 [SMILES: Cc1cc(=O)[nH]c2ccc(CC(=O)N3CCC(CCNC(=O)c4nccc5ccccc45)CC3)cc12] N-(2-(1-(2-(4-methyl-2-oxo-1,2-dihydroquinolin-6-yl)acetyl)piperidin-4- yl)ethyl)isoquinoline-1-carboxamide |
| 21 (CDD- 778) | 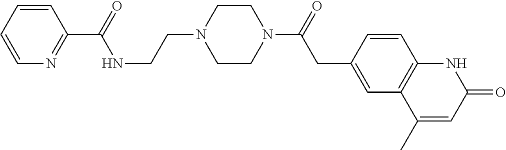 [SMILES: Cc1cc(=O)[nH]c2ccc(CC(=O)N3CCN(CCNC(=O)c4ccccn4)CC3)cc12] N-(2-(4-(2-(4-methyl-2-oxo-1,2-dihydroquinolin-6-yl)acetyl)piperazin-1- yl)ethyl)picolinamide |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from.

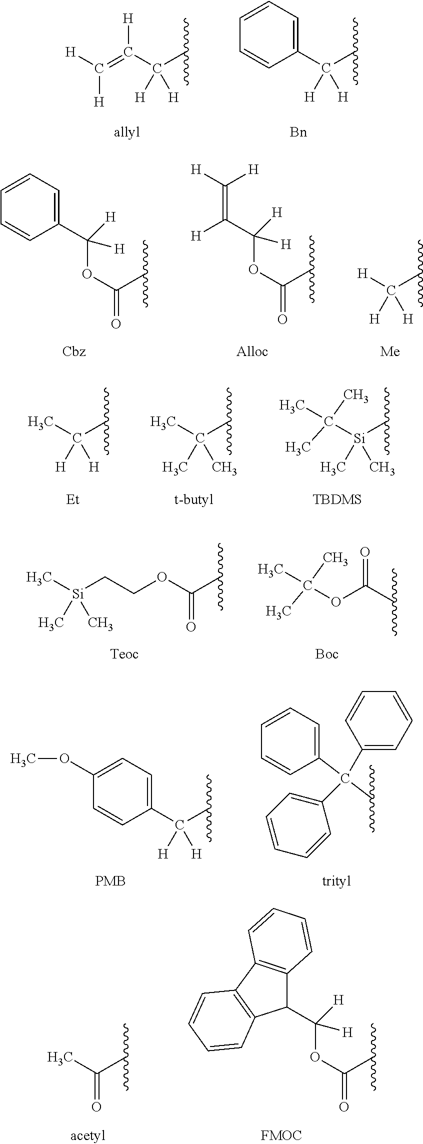

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Preparation of Compounds

In certain embodiments, a compound of formula (I) may be prepared according to Schemes 1-3.

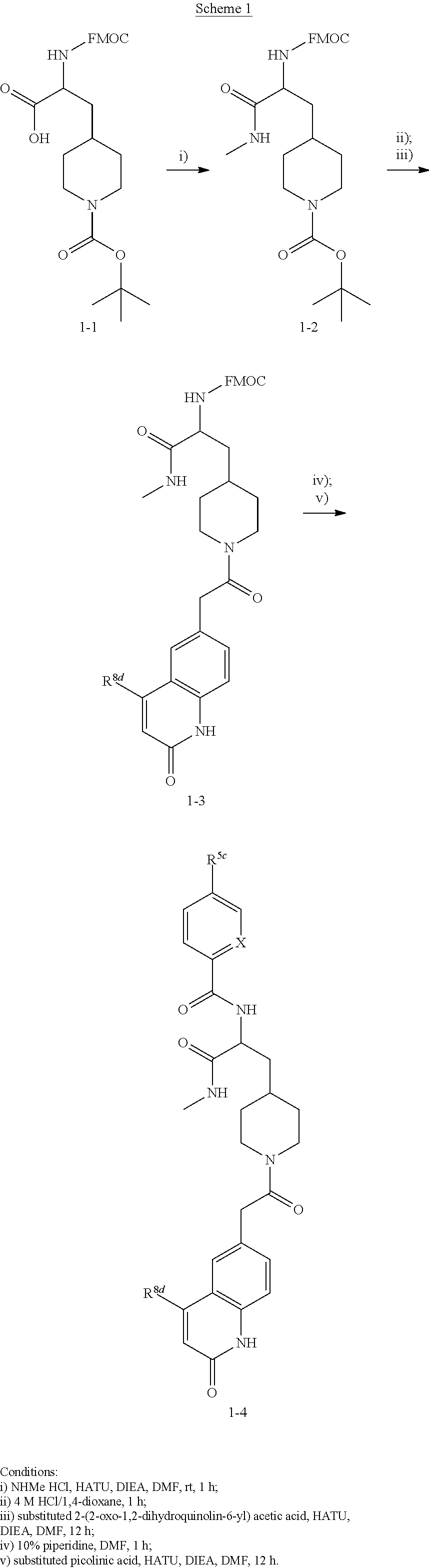

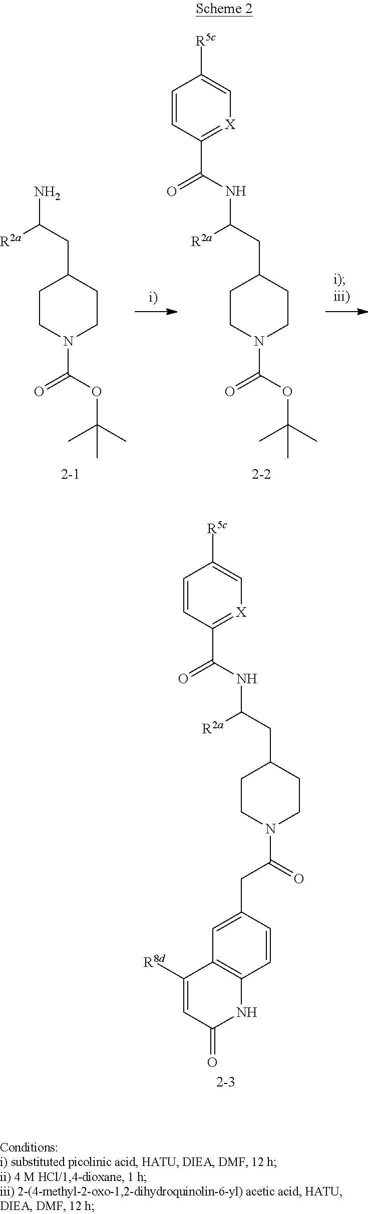

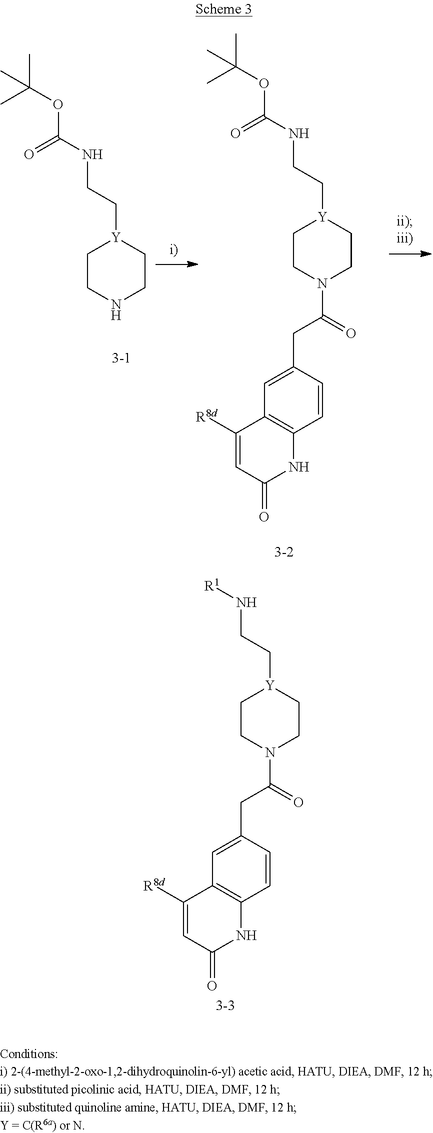

### Compositions

In one aspect, the present disclosure provides a pharmaceutical composition comprising the compound of the present disclosure and a pharmaceutically acceptable carrier.

The compositions containing the compound(s) described herein include a pharmaceutical composition comprising at least one compound as described herein and at least one pharmaceutically acceptable carrier. In some embodiments, the pharmaceutical composition comprises Kolliphor EL, and aqueous buffer, or a combination thereof. In certain embodiments, the aqueous buffer comprises phosphate buffered saline (PBS). In some embodiments, the aqueous buffer comprises 1×PBS. In certain embodiments, the pharmaceutical composition comprises about 5%, about 10%, about 15%, about 20%, about 25%, about 30%, about 35%, or about 40% Kolliphor EL. In some embodiments, the pharmaceutical composition comprises about 20% Kolliphor EL in 1×PBS.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In some embodiments, the composition is formulated as a pill, tablet, gelcap, or capsule for oral administration.

### Methods

In one aspect, the present disclosure provides a method of inhibiting bromodomain testis (BRDT) in a male subject. In certain embodiments, the method comprises administering to the male subject a therapeutically effective amount of at least one compound of the present disclosure, and/or the pharmaceutical composition of the present disclosure.

In another aspect, the present disclosure provides a method of promoting male contraception and/or infertility in a male subject. In certain embodiments, the method comprises administering to the male subject a therapeutically effective amount of at least one compound of the present disclosure, and/or the pharmaceutical composition of the present disclosure.

In another aspect, the present disclosure provides a method of minimizing and/or reducing spermatozoa number and/or motility in a male subject. In certain embodiments, the method comprises administering to the male subject a therapeutically effective amount of at least one compound of the present disclosure, and/or the pharmaceutical composition of the present disclosure.

In certain embodiments, the compound provides a contraceptive effect in the male subject.

In another aspect, the present disclosure provides a method of treating, preventing, and/or ameliorating cancer in a subject, the method comprising administering to the subject a therapeutically effective amount of at least one compound of the present disclosure and/or a pharmaceutical composition of the present disclosure.

In another aspect, the present disclosure provides a method of treating, preventing, and/or ameliorating an infectious disease in a subject, the method comprising administering to the subject a therapeutically effective amount of at least one compound of the present disclosure and/or a pharmaceutical composition of the present disclosure.

In certain embodiments, the infectious disease is a viral infection. In certain embodiments, the viral infection is caused by a coronavirus. In certain embodiments, the coronavirus is SARS-CoV-2.

In another aspect, the present disclosure provides a method of treating, preventing, and/or ameliorating an inflammatory condition in a subject, the method comprising administering to the subject a therapeutically effective amount of at least one compound of the present disclosure and/or a pharmaceutical composition of the present disclosure.

In another aspect, the present disclosure provides a method of treating, preventing, and/or ameliorating a metabolic disorder in a subject, the method comprising administering to the subject a therapeutically effective amount of at least one compound of the present disclosure and/or a pharmaceutical composition of the present disclosure.

In another aspect, the present disclosure provides a method of inhibiting BRD2, BRD3, BRD4, and/or BRDT in a subject with cancer, an inflammatory condition, an infectious disease, and/or metabolic disorder in which one or more of BET proteins are regulators, thereby treating, preventing, and/or ameliorating the condition, disease, and/or disorder, the method comprising administering to the subject a therapeutically effective amount of a compound of the disclosure and/or a pharmaceutical composition of the present disclosure.

In certain embodiments, the compound inhibits BRDT bromodomain-1 (BRDT-BD2).

In certain embodiments, the compound selectively inhibits BRDT-BD1 over BRDT bromodomain-2 (BRDT-BD2).

In certain embodiments, the compound inhibits BRD4 bromodomain-1 (BRD4-BD1).

In certain embodiments, the compound selectively inhibits BRD4-BD1 over BRD4 bromodomain-2 (BRD-BD2).

In certain embodiments, the compound inhibits BRD3 bromodomain-1 (BRD3-BD1).

In certain embodiments, the compound selectively inhibits BRD3-BD1 over BRD3 bromodomain-2 (BRD3-BD2).

In certain embodiments, the compound inhibits BRD2 bromodomain-1 (BRD2-BD1).

In certain embodiments, the compound selectively inhibits BRD2-BD1 over BRD2 bromodomain-2 (BRD2-BD2).

In certain embodiments, the subject is a mammal. In other embodiments, the mammal is a human.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional agent that inhibits BRDT, BRD4, BRD3, BRD2, or a combination thereof.

The compound of formula (I) can be administered to the subject using any administration route known to a person of skill in the art. Exemplary routes of administration are described elsewhere herein. In some embodiments, a composition comprising a compound of formula (I) is orally administered to the subject. In some embodiments, a pill, tablet, gelcap, or capsule comprising a compound of formula (I) is orally administered to the subject.

The compound of formula (I) can be administered to the subject in any dosage with any timing of dosage administration necessary to inhibit BRDT, BRD4, BRD3, BRD2, or a combination thereof and to provide a desired therapeutic effect. In some embodiments, the compound of formula (I) is administered to the male subject in order to provide a contraceptive effect in the subject. Although not wishing to be limited by theory, it is believed that inhibition of BRDT function in spermatocytes and spermatids leads to a reduction in spermatozoa number and motility. Therefore, in some embodiments, the dosage of the compound of formula (I) and the timing of dosage administration provide a contraceptive effect in the male subject. In some embodiments, the compound of formula (I) is administered to the subject daily. In certain embodiments, the compound of formula (I) is administered to the subject at a dosage of between about 10 mg/kg and 100 mg/kg, about 20 mg/kg and 90 mg/kg, about 30 mg/kg and 80 mg/kg, about 40 mg/kg and 70 mg/kg, or about 40 mg/kg and 60 mg/kg. In some embodiments, the compound of formula (I) is administered daily to the subject at a dosage of between about 40 mg/kg and 60 mg/kg to provide a contraceptive effect.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### Materials and Instrumentation

All reactions involving air-sensitive reagents were carried out in anhydrous solvents under an atmosphere of nitrogen. Reagents and solvents purchased from commercial supplies were used as received. Reactions were monitored by thin-layer chromatography (TLC) on Baker-flex® silica gel plates (IB2-F) using UV-light (254 and 365 nm) detection or high-performance liquid chromatography/mass spectrometry (HPLC-MS). Column chromatography was carried out using Teledyne ISCO CombiFlash system equipped with either a silica or C-18 column. NMR spectra were recorded at room temperature using a Bruker Avance III HD 600 MHz spectrometer (¹H NMR at 600 MHz and ¹³C NMR at 150 MHz) or a Bruker Avance III HD 800 MHz spectrometer (¹3C NMR at 200 MHz). Chemical shifts (δ) are reported in parts per million (ppm) with reference to solvent signals [¹H-NMR; DMSO-d₆ (2.50 ppm); ¹³C-NMR: DMSO-d₆ (39.51 ppm)]. Signal patterns are reported as s (singlet), d (doublet), t (triplet), q (quartet), h (heptet), m (multiplet) and br (broad). Coupling constants (J) are given in Hz. HRMS measurements were performed using ThermoFisher Scientific Q Exactive instrument.

Chiral analysis of compounds was performed using a Chiralpak_IH_3 column; Mobile phase: solvent A1 (heptane) and solvent B2 (ethanol); Gradient: isocratic, heptane/ethanol (40/60) for 15 minutes; Flow rate: 0.75 mL/min; Detection: UV 254 nm; Temperature: ambient; Concentration: 1 mM (injection volume 5 μL); Instrument: analytical HPLC.

### Production of Recombinant Bromodomain Proteins

Recombinant, His6-tagged BRDT-BD1 and BD2 were produced as previously described. In brief, cDNA encoding human BRDT and BRD4 BD1 and BD2 bromodomains subcloned into pET15b or pET28b bacterial vectors (Addgene, USA) with an N-terminal polyhistidine tag (6-His; linear) for expression and purification. His-tagged recombinant protein was isolated and eluted by immobilized metal affinity chromatograph followed by size exclusion gel filtration chromatography. Each BD was confirmed to be properly folded and active by a fluorescence thermal shift stability assay and AlphaScreen with biotinylated JQ1 as ligand, respectively.

Dec-Tec Affinity Selection with Bromodomain Proteins

To identify BRDT-BD1 selective compounds, the DEC-Tec library pool was screened. Five screening conditions were utilized: 1) absence of bromodomain proteins (bead binding non-target control, NTC), 2) presence of His-BRDT-BD1 at 0.3 μM, 3) presence of His-BRDT-BD2 at 0.3 μM (a counter-screen for bromodomain selective compounds), 4) presence of His-BRDT-BD1 plus JQ1 at 100 M, and 5) presence of His-BRDT-BD2 plus JQ1 at 100 μM. After three rounds of DEC-Tec selection, the DNA barcode from the last round of selection was PCR amplified. Following cleanup by Agencourt AMPure XP beads and quantitation with Agilent high sensitivity DNA kit using a Bioanalyzer, the DNA was sequenced in a single-read 105-cycle sequencing on an Illumina NextSeq 500 instrument.

### Bromodomain Proximity Assay

The AlphaScreen™ assay was performed following previous publication with minor modifications from the manufacturer's protocol (PerkinElmer, USA). A 20-μL reaction was set up in a PerkinElmer 384-well AlphaPlate where His-bromodomain at 10 nM was incubated with biotinylated JQ1 at 10 nM, nickel chelate acceptor beads at 12.5 g/mL, and tested compound at various concentrations for 15 min at room temperature, followed by the addition of streptavidin donor beads at 12.5 μg/mL and another 60-min incubation at room temperature. The plate was read on a Tecan Infinite M1000 Pro plate reader.

### Thermal Shift Assay

The dye SYPRO Orange (ThermoFisher Scientific, USA) was used to perform the protein thermal shift assay. The assay was set up on a 384-well Roche plate where His-bromodomain at a concentration of 2 μM was incubated with the test compound at various concentrations, and SYPRO Orange dye at 5× in a 10-μL reaction. The melting curve experiment and data analysis was run on a Roche Lightcycler 480 real-time PCR instrument.

### Metabolic Stability Assay in Liver Microsomes

Compounds 2 (CDD-787) and 4 (CDD-956) (2.0 M) were incubated in the mouse or human liver microsomes (0.5 mg protein/mL) at 37° C. in the presence of NADPH (1.0 mM). The samples were collected at specific time-points 0, 5, 10, 20, 40 and 60 min, in duplicate. The reactions were terminated by the addition of an equivalent volume of ice-cold CH₃OH, and subsequently vortexed. The reaction mixtures were centrifuged at 15,000 g for 15 min. Supernatant (5 μL) was analyzed by ultra-high performance liquid chromatography (UHPLC)-MS/MS analysis (QQQ, Agilent 6490) equipped with 50 mm×4.6 mm column (XDB C-18, Agilent Technologies, USA). LC-MS/MS (Agilent Technologies, QQQ, Santa Clara, CA) was operated in positive mode with electrospray ionization. Ultrapure nitrogen was applied as the sheath gas and the collision gas. The capillary gas temperature was set at 280° C. and the capillary voltage was set at 3.6 kV. The column temperature was maintained at 40° C. The flow rate was at 0.3 mL/min with a 50% mobile phase (acetonitrile containing 0.1% formic acid) in a 6-min run. Compounds 2 (CDD-787) and 4 (CDD-956) were measured using multiple-reaction monitoring method with the mass transition m/z 594.3>318.1 for 2; m/z 566.3>290.1 for 4. JQ1 (mass transition: m/z 457.4>341.3) and alprazolam (m/z 309.2>281.3) were used as the short and long half-life control, respectively.

### BROMOscan Bromodomain Profiling

BROMOscan bromodomain profiling was provided by Eurofins DiscoverX Corp. (San Diego, CA, USA, www.discoverx.com). Determination of the K_{d} between test compounds and DNA tagged bromodomains was achieved through binding competition against a proprietary reference immobilized ligand.

### BromoKdELECT Assays

Assays were performed at Eurofins DiscoverX (Fremont, CA) using the following procedures. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, and 1 mM DTT) to remove unbound ligand and to reduce non-specific phage binding. Binding reactions were assembled by combining bromodomains, liganded affinity beads, and test compounds in 1× binding buffer (16% SeaBlock, 0.32×PBS, 0.02% BSA, 0.04% Tween 20, 0.004% Sodium azide, and 7.9 mM DTT). Test compounds were prepared as 1000× stocks in 100% DMSO and subsequently diluted 1:25 in monoethylene glycol (MEG). The compounds were then diluted directly into the assays such that the final concentrations of DMSO and MEG were 0.1% and 2.4%, respectively. All reactions were performed in polypropylene 384 well plates in a final volume of 0.02 ml. The assay plates were incubated at room temperature with shaking for 1 hour and the affinity beads were washed with wash buffer (1×PBS with 0.05% Tween 20). The beads were then re-suspended in elution buffer (1×PBS with 0.05% Tween 20 and 2 μM of non-biotinylated affinity ligand) and incubated at room temperature with shaking for 30 minutes. The bromodomain concentration in the eluates was measured by qPCR.

### Crystallization, Data Collection, and Structure Determination

BRDT-BD1 was co-crystallized with compound 4 by the hanging drop vapor diffusion method. For crystallization, purified BRDT-BD1 at 3.5 mg/ml was mixed with a 5 molar excess of compound 4. The protein-inhibitor mixture was concentrated using Amicon Ultra-centrifugal filters (Millipore Sigma) to 24 mg/ml. Mosquito (TTP labtech) was used to dispense equal volumes of protein and reservoir (250 nl each) against 70 μl reservoir buffer in 96 wells crystallization tray (96-Well clear polystyrene microplate from SPT Labtech). Crystals of the BRDT-BD1/CDD-956 (compound 4) complex were observed after 2 days in drop containing 1.26 M Ammonium Sulfate, 0.1 M Tris (pH 8.5), and 0.2 M Lithium sulfate. The crystals were cryo-cooled in the same solution with 20% glycerol. The diffraction data were collected at Advanced Photo Source (APS) (Lemont, Illinois, USA). The data was integrated and sealed by using iMosfim and SCALA. The crystal structure of the BRDT-BD1/CDD-956 (compound 4) complex was determined by molecular replacement in PHENIX using a crystal structure of the human BRDT BD1 (PDB ID: 4FLP) as a search model. Compound 4 was fitted manually into electron density by using COOT. The final models have gone through several rounds of refinement using Phenix.refine, followed by manual model building using COOT. For all structural analysis and preparation of figures, the visualization program PyMOL was used.

### Nanobret Target Engagement Intracellular BET Bromodomain Assay

The NanoLuc-BRDT-BD1 and NanoLuc-BRDT-BD2 vectors were constructed by subcloning the same BRDT bromodomain sequences applied for protein expression into the NanoLuc-BRD4-BD1 and NanoLuc-BRD4-BD2 vectors (Promega, USA) to replace the corresponding BRD4 bromodomain sequences. The NanoBRET tracer competition assay was performed in transiently transfected HEK293 cells expressing each NanoLuc-bromodomain vector on a 384-well plate following the manufacturer's protocol (Promega, USA). Tracer titration was performed for each NanoLuc fusion vector to determine the optimized tracer concentration. The fusion protein was allowed to express for 36 h. The cells were then preincubated with tested compounds at different concentrations for 2 h followed by 2 h of incubation with tracer. Freshly prepared NanoBRET Nano-Glo substrate plus extracellular NanoLuc inhibitor were then added to initiate the subsequent bioluminescence resonance energy transfer (BRET) measurements using a CLARIOstar Plus BMG LABTECH plate reader. Data was analyzed by measuring the ratio of acceptor emission to donor emission (BRET ratio) and normalized by subtracting no-tracer-control-background.

### Cell Culture

AML cell lines MV4; 11, MOLM-13, THP-1, and Kasumil were obtained from the Texas Children's Hospital. Cell lines were incubated at 37° C. under 5% CO₂, in RPMI-1640 plus 10% FBS and 1% Penicillin/Streptomycin. Mycoplasma testing was performed using the LookOut Mycoplasma qPCR Detection Kit (Sigma) at entry into the lab. Cell line identity was confirmed annually using STR fingerprinting at the Cytogenetics and Cell Authentication Core at MD Anderson Cancer Center. Cells used for experiments were passaged less than 20 times thawing.

### Cell Viability Testing

Dose-response testing of hits were conducted on 40,000 cells/mL plated in 384-well plates with 4 ten-fold dilutions of tamibarotene and read out by CellTiterGlo (Promega) at 72 hours of treatment per manufacturer instructions. There were at least 3 biologic replicates for each condition, and experiments were independently repeated three times to confirm similar results. Analysis was done using GraphPad Prism normalizing to the CellTiterGlo signal of DMSO-treated samples as indicated.

### Flow Cytometry

Cells were harvested after 24 hours treatment for cell cycle analysis. After a PBS rinse, cells were fixed overnight in 70% methanol at −20° C. The following day, samples were rinsed with 0.5% BSA in PBS, then incubated in 100 g/mL RNAse/PBS at 37° C. for 15 minutes. Then cells were resuspended in 50 μg/mL PI in PBS. The samples were then analyzed on the BD canto II flow cytometer. For apoptosis detection, cells were harvested at 72 hours of treatment. Media was aspirated to leave a residual of 300-400 μL. DAPI and AlexaFluor 488-conjugated Annexin V antibody (1:200 dilution, ThermoFisher A13201) was added to the tube in equal volume, and samples were analyzed in the LSR I flow cytometer. For both experiments, 3 biologic replicates were averaged for each condition, and experiments were all repeated three times to confirm results. Results were analyzed using FlowJo v10 and plotted in GraphPad Prism using an unpaired t-test to compare statistical significance.

### Quantitative RT-PCR Analysis

Expression of MYC and GAPDH were determined using real-time quantitative reverse transcriptase-polymerase chain reaction (qPCR). N=3 technical replicates per sample and the experiment was performed at least once to confirm results on the same extracted RNA. Primers for MYC, GAPDH, and housekeeping control ACTB were ordered from Sigma-Millipore. Relative expression was calculated using the comparative ΔΔCt method normalizing to ACTB and DMSO. All results were plotted in GraphPad Prism using an unpaired t-test to determine statistical significance.

### Example 1: DEC-Tec Affinity Selection with Bromodomain Proteins

To identify small molecules that bind specifically to BRDT-BD1, fifty unique DNA-encoded chemical libraries (DECLs) cumulatively containing >4.5 billion drug-like compounds were pooled together for parallel screening of both BRDT-BD1 versus BRDT-BD2. Each library was first quantified by qPCR, and library pooling was conducted to have ˜1 million copies of each compound present in the pool. BRDT-BD1 and BRDT-BD2 were individually subjected to three rounds of affinity selection at a protein concentration of 0.3 μM. Two control affinity selections were performed in parallel: 1) without protein as a no-target control to identify any nonspecific bead binders, and 2) with the addition of 100 μM of the pan-bromodomain inhibitor JQ1 to identify competitive BRDT bromodomain binders.

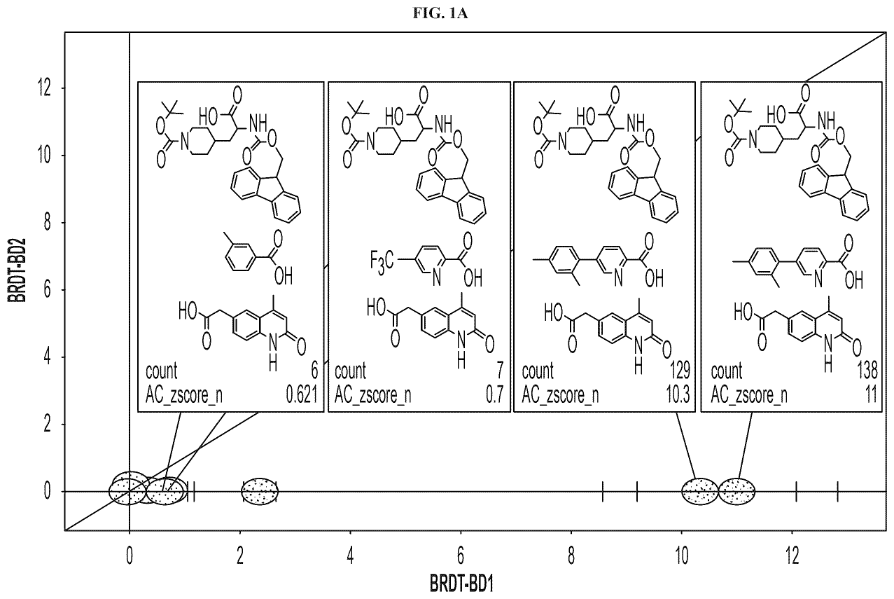

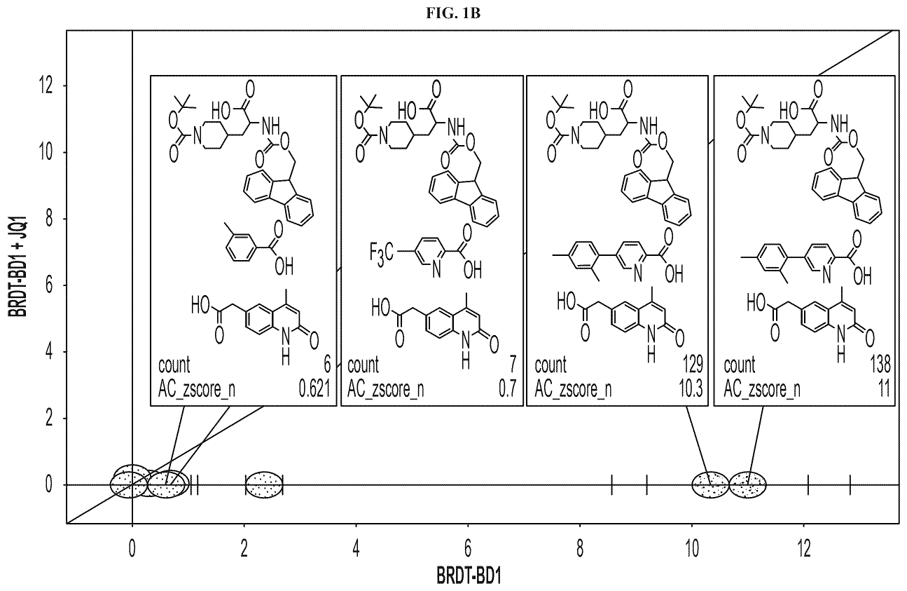

Illumina next-generation sequencing of the amplified, eluted binders of the targets identified a chemical series consistently enriched with excellent structure-enrichment relationships (SER) from one of the DEC-Tec libraries (FIGS. 1A-1B). As shown in FIGS. 1A-1B, strong multiple enrichments were identified with different DNA linkers; these hits had a good, normalized Z-score metric for BRDT-BD1 but not BRDT-BD2. These hits also demonstrated competition in the DEC-Tec screen with reference compound JQ1.

The observed chemical series contained propanoic acid substitution in cycle 1, substituted pyridine or phenyl as cycle 2, and 2-(2-oxo-1,2-dihydroquinolin-6-yl) acetic acid in cycle 3. From SER studies, different substitutions on pyridine cycle 2 resulted in different Z-scores. Altogether, the strong enrichment and reasonable SER suggested a promising 3-cycle chemical series and inspired further investigation of these compounds through synthesis of molecules off-DNA.

### Example 2: Compound Synthesis

### tert-butyl 4-(2-((((9H-fluoren-9-yl)methoxy)carbonyl)amino)-3-(methylamino)-3-oxopropyl)piperidine-1-carboxylate

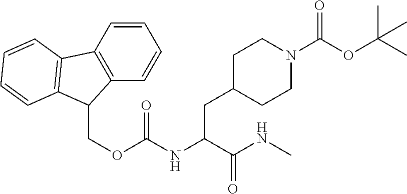 [SMILES: CNC(=O)C(CC1CCN(C(=O)OC(C)(C)C)CC1)NC(=O)OCC1c2ccccc2-c2ccccc21]

To a solution of 2-((((9H-fluoren-9-yl)methoxy)carbonyl)amino)-3-(1-(tert-butoxycarbonyl)piperidin-4-yl)propanoic acid (494.58 mg, 1 mmol, 1 equiv.), methylamine HCl (202.5 mg, 3 equiv.), and HATU (1140 mg, 3 mmol, 3 equiv.) in anhydrous DMF (10 mL) was added DIEA (522 μL, 3 mmol, 3 equiv.) under a nitrogen atmosphere. The reaction mixture was stirred at room temperature for 1 h and then quenched by the addition of water. The aqueous layer was extracted twice with EtOAc, and the combined organic extracts were washed with brine, dried over anhydrous Na₂SO₄, filtered and concentrated. The residue was purified by column chromatography on silica gel (CH₃OH/CH₂Cl₂, 1:99 to 5:95) to provide the title compound as an oil (507 mg, 98% yield). ¹H NMR (600 MHz, CD₃OD) δ 7.80 (d, J=7.5 Hz, 2H), 7.66 (t, J=7.9 Hz, 2H), 7.39 (t, J=7.3 Hz, 2H), 7.31 (t, J=7.4 Hz, 2H), 4.47 (dd, J=10.6, 6.7 Hz, 1H), 4.37 (dd, J=10.6, 6.5 Hz, 1H), 4.21 (t, J=6.5 Hz, 1H), 4.11 (dd, J=9.9, 4.9 Hz, 1H), 4.04-4.02 (m, 2H), 2.72-2.65 (m, 5H), 1.72-1.47 (m, 5H), 1.44 (s, 9H), 1.13-1.00 (m, 2H); HRMS (ESI) m/z calcd for C₂₉H₃₇N₃O₅ [M+H]+ 508.2806, found 508.2895.

### tert-butyl (R)-4-(2-((((9H-fluoren-9-yl)methoxy)carbonyl)amino)-3-(methylamino)-3-oxopropyl)piperidine-1-carboxylate

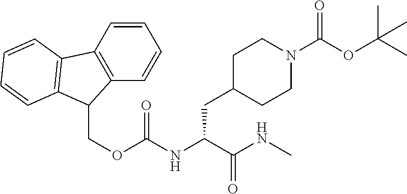 [SMILES: CNC(=O)[C@@H](CC1CCN(C(=O)OC(C)(C)C)CC1)NC(=O)OCC1c2ccccc2-c2ccccc21]

The title compound was prepared in a manner analogous to that which is described for tert-butyl 4-(2-((((9H-fluoren-9-yl)methoxy)carbonyl)amino)-3-(methylamino)-3-oxopropyl)piperidine-1-carboxylate (98% yield, white solid). ¹H NMR (600 MHz, CD₃OD) δ 7.80 (d, J=7.5 Hz, 2H), 7.66 (t, J=7.9 Hz, 2H), 7.39 (t, J=7.4 Hz, 2H), 7.31 (t, J=7.4 Hz, 2H), 4.48 (dd, J=10.6, 6.8 Hz, 1H), 4.37 (dd, J=10.6, 6.5 Hz, 1H), 4.22 (t, J=6.5 Hz, 1H), 4.11 (dd, J=9.9, 4.9 Hz, 1H), 4.04-4.02 (m, 2H), 2.71-2.65 (m, 5H), 1.73-1.47 (m, 5H), 1.44 (s, 9H), 1.13-1.00 (m, 2H); HRMS (ESI) m/z calcd for C₂₉H₃₇N₃O₅ [M+H]+ 508.2806, found 508.2794; [α]25/D+2.7±1°, c=1% in DMF.

### tert-butyl (S)-4-(2-((((9H-fluoren-9-yl)methoxy)carbonyl)amino)-3-(methylamino)-3-oxopropyl)piperidine-1-carboxylate

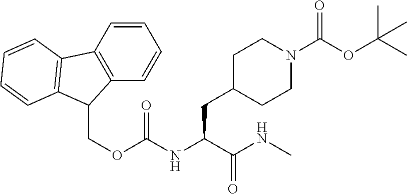 [SMILES: CNC(=O)[C@H](CC1CCN(C(=O)OC(C)(C)C)CC1)NC(=O)OCC1c2ccccc2-c2ccccc21]

The title compound was prepared in a manner analogous to that which is described for tert-butyl 4-(2-((((9H-fluoren-9-yl)methoxy)carbonyl)amino)-3-(methylamino)-3-oxopropyl)piperidine-1-carboxylate (98% yield, oil). ¹H NMR (600 MHz, CD₃OD) δ 7.80 (d, J=7.5 Hz, 2H), 7.66 (t, J=8.0 Hz, 2H), 7.39 (t, J=7.4 Hz, 2H), 7.31 (t, J=7.4 Hz, 2H), 4.48 (dd, J=10.6, 6.8 Hz, 1H), 4.37 (dd, J=10.6, 6.4 Hz, 1H), 4.22 (t, J=6.5 Hz, 1H), 4.11 (dd, J=9.9, 4.9 Hz, 1H), 4.04-4.02 (m, 2H), 2.71-2.65 (m, 5H), 1.72-1.47 (m, 5H), 1.44 (s, 9H), 1.13-1.00 (m, 2H); HRMS (ESI) m/z calcd for C₂₉H₃₇N₃O₅ [M+H]+ 508.2806, found 508.2800; [α]25/D−2.3±1°, c=1% in DMF.

### (9H-fluoren-9-yl)methyl (3-(1-(2-(4-methyl-2-oxo-1,2-dihydroquinolin-6-yl)acetyl)piperidin-4-yl)-1-(methylamino)-1-oxopropan-2-yl)carbamate

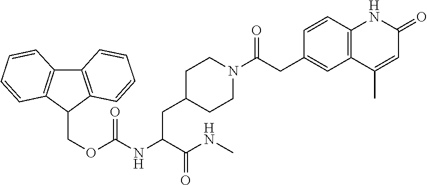 [SMILES: CNC(=O)C(CC1CCN(C(=O)Cc2ccc3[nH]c(=O)cc(C)c3c2)CC1)NC(=O)OCC1c2ccccc2-c2ccccc21]

To a mixture of tert-butyl 4-(2-((((9H-fluoren-9-yl)methoxy)carbonyl)amino)-3-(methylamino)-3-oxopropyl)piperidine-1-carboxylate (407 mg, 1 mmol, 0.6 eq) in 1,4-dioxane (20 mL) was added HCl/1,4-dioxane (4 M, 4 mL, 1.5 eq.) dropwise via syringe at 20° C., and the resulting reaction mixture stirred for 1 h. After completion of the reaction all solvents were removed in vacuo and kept overnight dry in high vacuo. To the same vessel was added a mixture of DIEA (522 L, 3 mmol, 2 eq), 2-(4-methyl-2-oxo-1,2-dihydroquinolin-6-yl) acetic acid (325 mg, 1.5 mmol, 1 eq), and HATU (1140 mg, 3 mmol, 3 equiv.) in anhydrous DMF (10 ml) under nitrogen, and the mixture was stirred for 12 h. The aqueous layer was extracted twice with EtOAc, and the combined organic extracts were washed with brine, dried over anhydrous Na₂SO₄, filtered and concentrated. The residue was purified by column chromatography on silica gel (CH₃OH/CH₂Cl₂, 1:99 to 5:95) to afford the title compound as a white solid (510 mg, 84% yield). ¹H NMR (600 MHz, DMSO-d₆) δ 11.57 (s, 1H), 7.89 (d, J=7.5 Hz, 2H), 7.85-7.84 (m, 1H), 7.72 (t, J=6.6 Hz, 2H), 7.53 (s, 1H), 7.51 (t, J=8.7 Hz, 1H), 7.42-7.40 (m, 2H), 7.36-7.30 (m, 3H), 7.24 (d, J=8.3 Hz, 1H), 6.39 (s, 1H), 6.28 (s, 1H), 4.36-4.28 (m, 2H), 4.25-4.20 (m, 2H), 3.99-3.96 (m, 2H), 3.76 (brs, 2H), 2.97-2.87 (m, 1H), 2.59-2.54 (m, 4H), 2.39 (s, 3H), 1.66-1.59 (m, 2H), 1.51-1.43 (m, 2H), 0.99-0.85 (m, 2H); HRMS (ESI) m/z calcd for C₃₆H₃₈N₄O₅ [M+H]+ 607.2915, found 607.2903.

### (9H-fluoren-9-yl)methyl (R)-(3-(1-(2-(4-methyl-2-oxo-1,2-dihydroquinolin-6-yl)acetyl)piperidin-4-yl)-1-(methylamino)-1-oxopropan-2-yl)carbamate

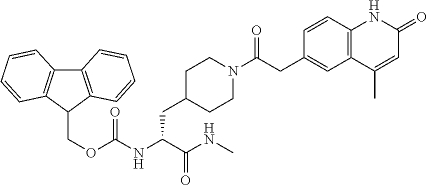 [SMILES: CNC(=O)[C@@H](CC1CCN(C(=O)Cc2ccc3[nH]c(=O)cc(C)c3c2)CC1)NC(=O)OCC1c2ccccc2-c2ccccc21]

The title compound was prepared in a manner analogous to that which is described for (9H-fluoren-9-yl)methyl (3-(1-(2-(4-methyl-2-oxo-1,2-dihydroquinolin-6-yl)acetyl)piperidin-4-yl)-1-(methylamino)-1-oxopropan-2-yl)carbamate (84% yield, white solid). ¹H NMR (600 MHz, DMSO-d₆) δ 11.57 (s, 1H), 7.89 (d, 0.1=7.5 Hz, 2H), 7.85-7.83 (m, 1H), 7.72 (t, 0.1=6.6 Hz, 2H), 7.53 (s, 1H), 7.50 (t, J=8.7 Hz, 1H), 7.43-7.40 (m, 2H), 7.36-7.30 (m, 3H), 7.24 (d, J=8.3 Hz, 1H), 6.39 (s, 1H), 6.28 (s, 1H), 4.36-4.30 (m, 2H), 4.25-4.20 (m, 2H), 3.98-3.96 (m, 2H), 3.76 (brs, 2H), 2.97-2.87 (m, 1H), 2.58-2.54 (m, 4H), 2.39 (s, 3H), 1.66-1.59 (m, 2H), 1.51-1.40 (m, 2H), 0.97-0.84 (m, 2H); HRMS (ESI) m/z calcd for C₃₆H₃₈N₄O₅ [M+H]+ 607.2915, found 607.2912; [α]25/D+1.7+1°, c=1% in DMF.

### (9H-fluoren-9-yl)methyl (S)-(3-(1-(2-(4-methyl-2-oxo-1,2-dihydroquinolin-6-yl)acetyl)piperidin-4-yl)-1-(methylamino)-1-oxopropan-2-yl)carbamate

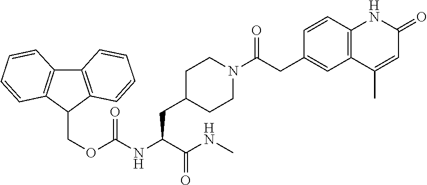 [SMILES: CNC(=O)[C@H](CC1CCN(C(=O)Cc2ccc3[nH]c(=O)cc(C)c3c2)CC1)NC(=O)OCC1c2ccccc2-c2ccccc21]

The title compound was prepared in a manner analogous to that which is described for (9H-fluoren-9-yl)methyl (3-(1-(2-(4-methyl-2-oxo-1,2-dihydroquinolin-6-yl)acetyl)piperidin-4-yl)-1-(methylamino)-1-oxopropan-2-yl)carbamate (84% yield, oil). ¹H NMR (600 MHz, DMSO-d₆) δ 11.57 (s, 1H), 7.89 (d, J=7.5 Hz, 2H), 7.84-7.83 (m, 1H), 7.72 (t, J=6.6 Hz, 2H), 7.53 (s, 1H), 7.50 (t, J=8.7 Hz, 1H), 7.42-7.39 (m, 2H), 7.35-7.30 (m, 3H), 7.24 (d, J=8.3 Hz, 1H), 6.38 (s, 1H), 6.27 (s, 1H), 4.35-4.29 (m, 2H), 4.24-4.19 (m, 2H), 3.99-3.93 (m, 2H), 3.75 (brs, 2H), 2.96-2.86 (m, 1H), 2.57-2.55 (m, 4H), 2.38 (s, 3H), 1.67-1.60 (m, 2H), 1.50-1.38 (m, 2H), 0.96-0.84 (m, 2H); HRMS (ESI) m/z calcd for C₃₆H₃₈N₄O₅ [M+H]+ 607.2915, found 607.2902; [α]25/D−1.0±1°, c=1% in DMF.

### (9H-fluoren-9-yl)methyl (R)-(1-(methylamino)-1-oxo-3-(1-(2-(2-oxo-1,2-dihydroquinolin-6-yl)acetyl)piperidin-4-yl)propan-2-yl)carbamate

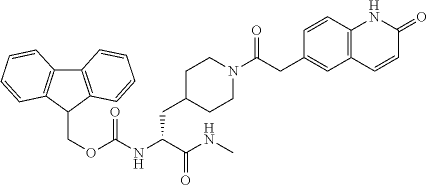 [SMILES: CNC(=O)[C@@H](CC1CCN(C(=O)Cc2ccc3[nH]c(=O)ccc3c2)CC1)NC(=O)OCC1c2ccccc2-c2ccccc21]

The title compound was prepared in a manner analogous to that which is described for (9H-fluoren-9-yl)methyl (3-(1-(2-(4-methyl-2-oxo-1,2-dihydroquinolin-6-yl)acetyl)piperidin-4-yl)-1-(methylamino)-1-oxopropan-2-yl)carbamate (84% yield, white solid). ¹H NMR (600 MHz, CD₃OD) δ 7.88-7.87 (m, 1H), 7.75 (d, J=7.1 Hz, 2H), 7.61 (brs, 2H), 7.49 (brs, 1H), 7.42 (d, J=6.6 Hz, 2H), 7.37-7.26 (m, 5H), 6.6 (m, 1H), 4.52-4.44 (m, 2H), 4.37-4.33 (m, 1H), 4.18 (t, J=6.4 Hz, 1H), 4.11-4.08 (m, 1H), 3.96 (brs, 1H), 3.80 (s, 2H), 3.03-2.93 (m, 1H), 2.70 (s, 3H), 2.63-2.55 (m, 1H), 1.78-1.52 (m, 5H), 1.10-0.87 (m, 2H); HRMS (ESI) m/z calcd for C₃₅H₃₆N₄O₅ [M+H]+ 593.2758, found 593.2754; [α]25/D+1.3+1°, c=1% in DMF.

### 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (CDD-724) (1)

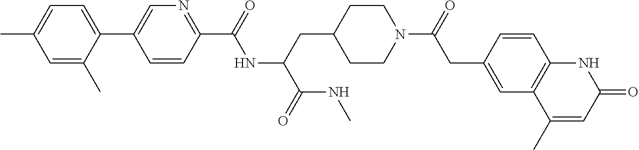 [SMILES: CNC(=O)C(CC1CCN(C(=O)Cc2ccc3[nH]c(=O)cc(C)c3c2)CC1)NC(=O)c1ccc(-c2ccc(C)cc2C)cn1]

(9H-fluoren-9-yl)methyl (3-(1-(2-(4-methyl-2-oxo-1,2-dihydroquinolin-6-yl)acetyl)piperidin-4-yl)-1-(methylamino)-1-oxopropan-2-yl)carbamate (322 mg, 0.84 mmol, 1 eq) was dissolved in DMF (3 mL) containing piperidine (10% v/v), and stirred for 1 h at room temperature. Next, all solvents were removed in vacuo and placed under high vacuum overnight. To the same vessel was added a mixture of DIEA (439 μL, 2.5 mmol, 3 eq), 5-(2,4-dimethylphenyl) picolinic acid (140 mg, 0.7 mmol, 0.8 eq) and HATU (957.6 mg, 2.5 mmol, 3 equiv.) in anhydrous DMF (8 mL) under a nitrogen atmosphere. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to afford the title compound as a white solid (327 mg, 68%). ¹H NMR (600 MHz, DMSO-d₆) δ 11.56 (s, 1H), 8.66 (t, J=9.4 Hz, 1H), 8.63 (s, 1H), 8.10-8.08 (m, 2H), 7.98 (dd, J=7.9, 2.1 Hz, 1H), 7.53 (brs, 1H), 7.34 (d, J=7.9 Hz, 1H), 7.23 (d, J=8.3 Hz, 1H), 7.18-7.17 (m, 2H), 7.14 (d, J=7.8 Hz, 1H), 6.38 (s, 1H), 4.58-4.56 (m, 1H), 4.36-4.31 (m, 1H), 3.99-3.93 (m, 1H), 3.75 (s, 2H), 2.97-2.91 (m, 1H), 2.6 (d, J=4.5 Hz, 2H), 2.50-2.46 (m, 1H), 2.39 (d, J=3.1 Hz, 3H), 2.33 (s, 3H), 2.23 (s, 3H), 1.8 (brs, 1H), 1.69-1.52 (m, 4H), 0.97-0.94 (m, 2H); 13C NMR (150 MHz, DMSO-d₆) δ 171.8, 168.4, 163.1, 161.5, 148.3, 147.6, 147.6, 139.6, 138.0, 137.8, 137.2, 134.9, 134.0, 131.4, 131.3, 129.7, 129.4, 126.9, 124.7, 121.5, 120.9, 119.4, 115.3, 50.3, 45.4, 41.3, 32.6, 32.1, 31.9, 31.5, 30.9, 25.6, 20.7, 19.9, 18.5; HRMS (ESI) m/z calcd for C₃₅H₃₉N₅O₄ [M+H]+ 594.3075, found 594.3068. Chiral HPLC: Enantiomer I (50.651%, tᵣ=7.522 min); Enantiomer II (49.349%, tᵣ=12.01 min).

### (R)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide (CDD-787) (2)

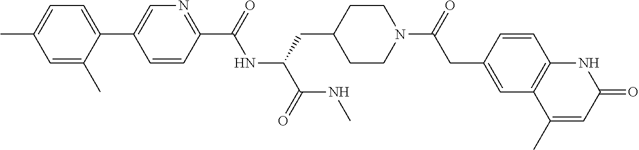 [SMILES: CNC(=O)[C@@H](CC1CCN(C(=O)Cc2ccc3[nH]c(=O)cc(C)c3c2)CC1)NC(=O)c1ccc(-c2ccc(C)cc2C)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (60% yield, white solid). ¹H NMR (600 MHz, DMSO-d₆) δ 11.56 (s, 1H), 8.66 (t, J=9.4 Hz, 1H), 8.63 (s, 1H), 8.10-8.08 (m, 2H), 7.98 (dd, J=7.9, 2.1 Hz, 1H), 7.53 (brs, 1H), 7.34 (d, J=7.9 Hz, 1H), 7.23 (d, J=8.3, Hz, 1H), 7.19-7.18 (m, 2H), 7.14 (d, J=7.8 Hz, 1H), 6.38 (s, 1H), 4.58-4.56 (m, 1H), 4.36-4.31 (m, 1H), 3.99-3.93 (m, 1H), 3.75 (s, 2H), 2.98-2.91 (m, 1H), 2.6 (d, J=4.5 Hz, 2H), 2.50-2.46 (m, 1H), 2.39 (d, J=3.1 Hz, 3H), 2.33 (s, 3H), 2.23 (s, 3H), 1.8 (brs, 1H), 1.71-1.52 (m, 4H), 0.99-0.95 (m, 2H); 13C NMR (150 MHz, DMSO-d₆) δ 171.8, 168.5, 163.2, 161.5, 148.4, 147.7, 139.6, 138.0, 137.8, 137.2, 134.9, 134.0, 131.3, 129.7, 129.4, 126.9, 124.7, 121.5, 120.9, 119.4, 115.3, 50.3, 45.4, 41.3, 32.6, 32.1, 31.9, 31.6, 30.9, 25.6, 20.7, 19.9, 18.5; HRMS (ESI) m/z calcd for C₃₅H₃₉N₅O₄ [M+H]+ 594.3075, found 594.3072; [α]25/D+16.2±1°, c=1% in DMF. Chiral HPLC: Enantiomer I (not observed); Enantiomer II (100%, tᵣ=12.102)
(S)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide (CDD-786) (3)

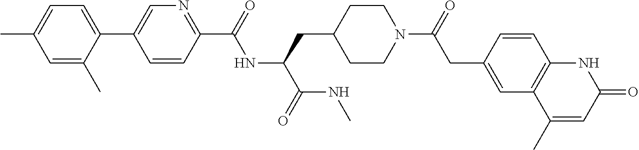 [SMILES: CNC(=O)[C@H](CC1CCN(C(=O)Cc2ccc3[nH]c(=O)cc(C)c3c2)CC1)NC(=O)c1ccc(-c2ccc(C)cc2C)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (60% yield, white solid). ¹H NMR (600 MHz, DMSO-d₆) δ 11.55 (s, 1H), 8.66 (t, J=9.4 Hz, 1H), 8.63 (s, 1H), 8.09-8.08 (m, 2H), 7.98 (dd, J=7.9, 2.1 Hz, 1H), 7.53 (brs, 1H), 7.34 (d, J=7.9 Hz, 1H), 7.23 (d, J=8.3, Hz, 1H), 7.18-7.17 (m, 2H), 7.13 (d, J=7.8 Hz, 1H), 6.38 (s, 1H), 4.59-4.54 (m, 1H), 4.36-4.31 (m, 1H), 3.98-3.92 (m, 1H), 3.75 (s, 2H), 2.98-2.89 (m, 1H), 2.59 (d, J=4.5 Hz, 2H), 2.54-2.46 (m, 1H), 2.38 (d, J=3.1 Hz, 3H), 2.33 (s, 3H), 2.22 (s, 3H), 1.8 (brs, 1H), 1.72-1.52 (m, 4H), 0.99-0.93 (m, 2H); 13C NMR (150 MHz, DMSO-d₆) δ 171.8, 168.4, 163.1, 161.5, 148.3, 147.6, 147.5, 139.6, 138.0, 137.8, 137.2, 134.9, 134.0, 131.4, 131.3, 129.7, 129.4, 126.9, 124.7, 121.5, 120.9, 119.4, 115.3, 50.3, 45.4, 41.3, 32.6, 32.1, 31.9, 31.5, 30.9, 25.6, 20.7, 19.9, 18.5; HRMS (ESI) m/z calcd for C₃₅H₃₉N₅O₄ [M+H]+ 594.3075, found 594.3073; [α]25/D−16.7±1°, c=1% in DMF. Chiral HPLC: Enantiomer I (100.0%, tᵣ=7.595 min); Enantiomer II (not observed).

### (R)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide (CDD-956) (4)

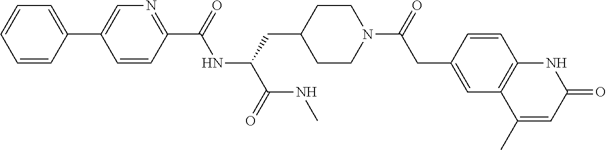 [SMILES: CNC(=O)[C@@H](CC1CCN(C(=O)Cc2ccc3[nH]c(=O)cc(C)c3c2)CC1)NC(=O)c1ccc(-c2ccccc2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (60% yield, white solid). ¹H NMR (600 MHz, DMSO-d₆) δ 11.54 (s, 1H), 8.98 (s, 1H), 8.65 (t, J=9.0 Hz, 1H), 8.29 (dd, J=8.1, 2.1 Hz, 1H), 8.12-8.09 (m, 2H), 7.80 (d, J=7.3 Hz, 2H), 7.56-7.53 (m, 3H), 7.4 (t, J=7.3 Hz, 1H), 7.34 (d, J=8.3 Hz, 1H), 7.23 (d, J=8.3 Hz, 1H), 6.38 (s, 1H), 4.61-4.56 (m, 1H), 4.36-4.32 (m, 1H), 3.98-3.93 (m, 1H), 3.77-3.72 (m, 2H), 2.97-2.89 (m, 1H), 2.61 (d, J=4.5 Hz, 1H), 2.54-2.46 (m, 1H), 2.38 (d, J=2.5 Hz, 3H), 1.80 (s, 1H), 1.73-1.52 (m, 4H), 0.99-0.93 (m, 2H); 13C NMR (150 MHz, DMSO-d₆) δ 171.7, 168.4, 163.0, 161.4, 148.1, 147.5, 146.5, 138.1, 137.1, 136.1, 135.5, 131.2, 129.3, 129.2, 128.7, 127.1, 124.6, 122.0, 120.8, 119.3, 115.2, 50.2, 45.3, 41.2, 32.5, 32.0, 31.8, 31.5, 30.9, 25.5, 18.4; HRMS (ESI) m/z calcd for C₃₃H₃₅N₅O₄ [M+H]+ 566.2762, found 566.2762; [α]25/D+19.1±1°, c=1% in DMF. Chiral HPLC: Enantiomer I (1.919%, tᵣ=2.78 min); Enantiomer II (98.081%, tᵣ=5.137 min).

### (S)-N-(3-(1-(2-(4-methyl-2-oxo-1,2-dihydroquinolin-6-yl) acetyl) piperidin-4-yl)-1-(methylamino)-1-oxo propan-2-yl)-5-phenylpicolinamide (CDD-2107) (5)

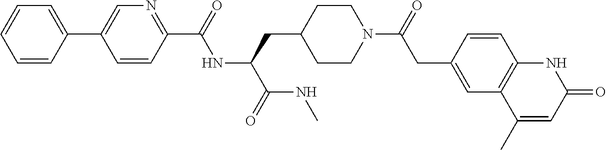 [SMILES: CNC(=O)[C@H](CC1CCN(C(=O)Cc2ccc3[nH]c(=O)cc(C)c3c2)CC1)NC(=O)c1ccc(-c2ccccc2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (55% yield, white solid). ¹H NMR (600 MHz, CD₃OD) δ 8.88 (s, 1H), 8.18 (dd, J=8.0, 1.5 Hz, 1H), 8.14-8.12 (m, 1H), 7.69 (d, J=7.3 Hz, 2H), 7.66 (s, 1H), 7.51 (t, J=7.5 Hz, 2H), 7.44 (t, J=8.3 Hz, 2H), 7.31 (d, J=8.4 Hz, 1H), 6.49 (s, 1H), 4.69-4.64 (m, 1H), 4.52-4.51 (m, 1H), 4.05-4.03 (m, 1H), 3.89-3.85 (m, 2H), 3.07-2.99 (m, 1H), 2.65-2.57 (m, 1H), 2.49 (s, 3H), 1.92-1.65 (m, 5H), 1.18-0.95 (m, 2H); 13C NMR (150 MHz, CD₃OD) δ 174.6, 171.6, 166.2, 164.8, 151.4, 149.1, 148.0, 138.2, 137.9, 136.8, 132.8, 131.5, 130.3, 129.9, 128.3, 125.9, 125.9, 125.9, 123.4, 121.9, 120.9, 117.4, 52.3, 47.5, 43.4, 40.6, 33.9, 33.3, 32.9, 32.3, 26.4, 19.1; HRMS (ESI) m/z calcd for C₃₃H₃₅N₅O₄ [M+H]+ 566.2762, found 566.2748; [α]25/D−17.7±1°, c=1% in DMF. Chiral HPLC: Enantiomer I (100%, tᵣ=5.137); Enantiomer II (not observed).

### (R)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide (CDD-818) (6)

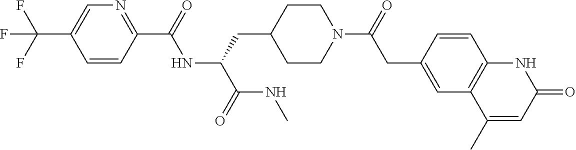 [SMILES: CNC(=O)[C@@H](CC1CCN(C(=O)Cc2ccc3[nH]c(=O)cc(C)c3c2)CC1)NC(=O)c1ccc(C(F)(F)F)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (65% yield, white solid). ¹H NMR (600 MHz, CD₃OD) δ 8.92 (s, 1H), 8.28-8.22 (m, 2H), 7.60 (s, 1H), 7.39 (d, J=8.2 Hz, 1H), 7.25 (d, J=8.3 Hz, 1H), 6.44 (s, 1H), 4.70-4.65 (m, 1H), 4.52-4.50 (m, 1H), 4.05-4.03 (m, 1H), 3.87-3.80 (m, 2H), 3.06-2.96 (m, 1H), 2.73 (s, 3H), 2.61-2.55 (m, 1H), 2.45 (s, 3H), 1.90-1.64 (m, 5H), 1.14-0.98 (m, 2H); 13C NMR (150 MHz, DMSO-d₆) δ 174.4, 171.5, 164.9, 153.8, 151.1, 146.6, 138.1, 136.4, 132.7, 131.3, 130.0, 129.8, 125.9, 123.4, 121.7, 121.0, 117.3, 52.4, 47.3, 43.3, 40.6, 40.0, 33.9, 33.3, 32.9, 32.2, 26.4, 19.0; HRMS (ESI) m/z calcd for C₂₈H₃₀F₃N₅O₄ [M+H]+ 558.2323, found 558.2310; [α]25/D+8.5+10, c=1% in DMF.

### (S)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide (CDD-819) (7)

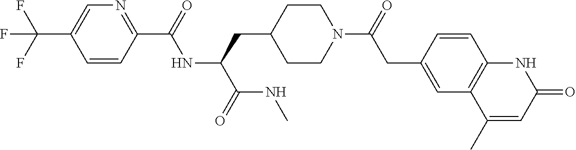 [SMILES: CNC(=O)[C@H](CC1CCN(C(=O)Cc2ccc3[nH]c(=O)cc(C)c3c2)CC1)NC(=O)c1ccc(C(F)(F)F)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (80% yield, white solid). ¹H NMR (600 MHz, CD₃OD) δ 8.95 (s, 1H), 8.31-8.21 (m, 2H), 7.66 (s, 1H), 7.43 (d, J=8.4 Hz, 1H), 7.30 (d, J=8.4 Hz, 1H), 6.49 (s, 1H), 4.68-4.63 (m, 1H), 4.53-4.51 (m, 1H), 4.06-4.04 (m, 1H), 3.89-3.82 (m, 2H), 3.07-2.99 (m, 1H), 2.73 (s, 3H), 2.63-2.56 (m, 1H), 2.49 (s, 3H), 1.91-1.63 (m, 5H), 1.15-0.96 (m, 2H); 13C NMR (150 MHz, CD₃OD) δ 174.4, 171.6, 164.8, 153.8, 151.3, 146.7, 138.2, 136.4, 132.7, 131.4, 130.1, 129.9, 125.9, 123.4, 121.9, 121.0, 117.4, 52.4, 47.5, 43.3, 40.7, 40.0, 33.9, 33.3, 32.9, 32.2, 26.4, 19.0; HRMS (ESI) m/z calcd for C₂₈H₃₀F₃N₅O₄ [M+H]+ 558.2323, found 558.2309; [α]25/D−7.5±1°, c=1% in DMF.

### (R)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (CDD-981) (8)

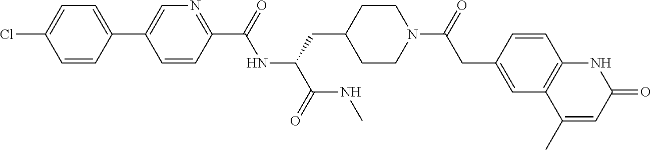 [SMILES: CNC(=O)[C@@H](CC1CCN(C(=O)Cc2ccc3[nH]c(=O)cc(C)c3c2)CC1)NC(=O)c1ccc(-c2ccc(Cl)cc2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (55% yield, white solid). ¹H NMR (600 MHz, CD₃OD) δ 8.82 (s, 1H), 8.14-8.09 (m, 2H), 7.65 (d, J=8.5 Hz, 2H), 7.61 (s, 1H), 7.47 (d, J=8.5 Hz, 2H), 7.39 (d, J=8.4 Hz, 1H), 7.26 (d, J=8.4 Hz, 1H), 6.45 (s, 1H), 4.69-4.64 (m, 1H), 4.53-4.50 (m, 1H), 4.04-4.02 (m, 1H), 3.87-3.80 (m, 2H), 3.05-2.97 (m, 1H), 2.74 (s, 3H), 2.64-2.56 (m, 1H), 2.45 (s, 3H), 1.91-1.64 (m, 5H), 1.16-0.95 (m, 2H); 13C NMR (150 MHz, CD₃OD) δ 174.6, 171.5, 166.0, 164.7, 151.2, 149.4, 147.9, 139.4, 138.1, 136.6, 136.5, 136.1, 132.7, 131.3, 130.4, 129.8, 125.9, 123.4, 121.8, 121.0, 117.3, 52.3, 47.4, 43.3, 40.6, 40.2, 33.9, 33.3, 32.9, 32.3, 26.4, 19.0; HRMS (ESI) m/z calcd for C₃₃H₃₅ClN₅O₄ [M+H]+ 600.2372, found 600.2343; [α]25/D+16.5±1°, c=1% in DMF.

### (R)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (CDD-982) (9)

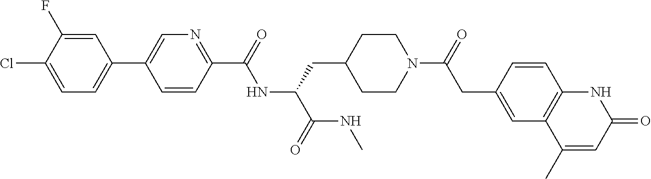 [SMILES: CNC(=O)[C@@H](CC1CCN(C(=O)Cc2ccc3[nH]c(=O)cc(C)c3c2)CC1)NC(=O)c1ccc(-c2ccc(Cl)c(F)c2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (58% yield, white solid). ¹H NMR (600 MHz, CD₃OD) δ 8.87 (s, 1H), 8.18 (dd, J=8.1, 1.0 Hz, 1H), 8.13-8.11 (m, 1H), 7.63-7.61 (m, 2H), 7.58 (t, J=7.9 Hz, 1H), 7.51 (dd, J=8.3, 1.4 Hz, 1H), 7.41 (d, J=8.4 Hz, 1H), 7.28 (d, J=8.4 Hz, 1H), 6.4 (brs, 1H), 4.69-4.64 (m, 1H), 4.53-4.51 (m, 1H), 4.05-4.03 (m, 1H), 3.88-3.81 (m, 2H), 3.06-2.99 (m, 1H), 2.74 (s, 3H), 2.64-2.57 (m, 1H), 2.47 (s, 3H), 1.92-1.65 (m, 5H), 1.17-0.96 (m, 2H); 13C NMR (150 MHz, CD₃OD) δ 174.6, 171.6, 165.9, 164.8, 160.6, 158.9, 151.2, 149.8, 147.9, 138.8, 138.2, 136.8, 132.5, 131.4, 125.9, 125.0, 123.4, 122.4, 121.8, 121.0, 117.3, 116.5, 52.3, 43.4, 40.6, 40.2, 33.9, 33.3, 33.0, 32.3, 26.4, 19.0; HRMS (ESI) m/z calcd for C₃₃H₃₃ClFN₅O₄ [M+H]+ 618.2278, found 618.2275; [α]25/D+15.8+1°, c=1% in DMF.

### (R)-N-(1-(methylamino)-1-oxo-3-(1-(2-(2-oxo-1,2-dihydroquinolin-6-yl)acetyl)piperidin-4-yl)propan-2-yl)-5-phenylpicolinamide (CDD-986) (10)

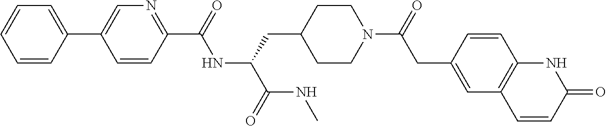 [SMILES: CNC(=O)[C@@H](CC1CCN(C(=O)Cc2ccc3[nH]c(=O)ccc3c2)CC1)NC(=O)c1ccc(-c2ccccc2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (58% yield, white solid). ¹H NMR (600 MHz, CD₃OD) δ 8.88 (s, 1H), 8.19-8.12 (m, 2H), 7.9 (d, J=8.4 Hz, 1H), 7.69 (d, J=7.3 Hz, 2H), 7.52-7.49 (m, 3H), 7.45-7.42 (m, 2H), 7.31 (d, J=9.0 Hz, 1H), 6.59 (d, J=9.4 Hz, 1H), 4.69-4.64 (m, 1H), 4.53-4.47 (m, 1H), 4.01 (t, J=8.3 Hz, 1H), 3.82 (s, 2H), 3.06-2.97 (m, 1H), 2.74 (s, 3H), 2.61-2.54 (m, 1H), 1.92-1.64 (m, 5H), 1.15-0.96 (m, 2H); 13C NMR (150 MHz, CD₃OD) δ 174, 171.4, 166.2, 165.1, 149.1, 148.0, 142.5, 140.9, 138.5, 137.9, 136.7, 132.8, 131.5, 130.3, 129.0, 128.3, 123.4, 122.0, 121.4, 116.9, 52.3, 47.5, 43.3, 40.5, 40.3, 33.9, 33.2, 32.9, 32.2, 26.4; HRMS (EST) m/z calcd for C₃₂H₃₃N₅O₄ [M+H]+ 552.2605, found 552.2601; [α]25/D+10.7±1°, c=1% in DMF.

### N-(3-(1-(2-(4-methyl-2-oxo-1,2-dihydroquinolin-6-yl)acetyl)piperidin-4-yl)-1-(methylamino)-1-oxopropan-2-yl)picolinamide (CDD-784) (11)

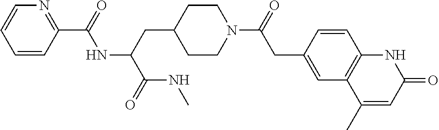 [SMILES: CNC(=O)C(CC1CCN(C(=O)Cc2ccc3[nH]c(=O)cc(C)c3c2)CC1)NC(=O)c1ccccn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (72% yield, white solid). ¹H NMR (600 MHz, DMSO-d₆) δ 11.58 (s, 1H), 8.67 (d, J=4.5 Hz, 1H), 8.63 (t, J=8.5 Hz, 1H), 8.08-8.00 (m, 3H), 7.64-7.62 (m, 1H), 7.53 (s, 1H), 7.34 (d, J=8.3 Hz, 1H), 7.24 (d, J=8.3 Hz, 1H), 6.38 (brs, 1H), 4.56-4.53 (m, 1H), 4.35-4.30 (m, 1H), 3.97-3.92 (m, 1H), 3.74 (s, 2H), 2.96-2.88 (m, 1H), 2.59 (d, J=4.5 Hz, 3H), 2.54 (s, 2H), 2.39 (brs, 3H), 1.78-1.76 (m, 1H), 1.69-1.50 (m, 4H), 1.01-0.90 (m, 2H); 13C NMR (150 MHz, DMSO-d₆) δ 172.2, 168.9, 162.0, 149.7, 148.9, 138.4, 137.6, 131.8, 129.9, 127.2, 125.1, 122.4, 121.2, 119.9, 116.6, 50.7, 45.8, 41.7, 40.9, 33.0, 32.5, 32.3, 32.0, 31.4, 26.0, 18.9; HRMS (ESI) m/z calcd for C₂₇H₃₁N₅O₄ [M+H]+ 490.2449, found 490.2451.

### N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide (CDD-785) (12)

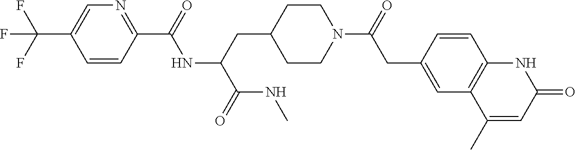 [SMILES: CNC(=O)C(CC1CCN(C(=O)Cc2ccc3[nH]c(=O)cc(C)c3c2)CC1)NC(=O)c1ccc(C(F)(F)F)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (72% yield, white solid). ¹H NMR (600 MHz, DMSO-d₆) δ 11.55 (s, 1H), 9.08 (s, 1H), 8.76 (t, J=9.2 Hz, 1H), 8.43 (d, J=8.2 Hz, 1H), 8.23 (dd, J=7.9, 5.1 Hz, 1H), 8.07 (s, 1H), 7.52 (s, 1H), 7.33 (d, J=8.3 Hz, 1H), 7.23 (d, J=8.3 Hz, 1H), 6.38 (s, 1H), 4.59-4.53 (m, 1H), 4.35-4.31 (m, 1H), 3.97-3.92 (m, 1H), 3.74 (s, 2H), 2.96-2.87 (m, 1H), 2.59 (d, J=4.5 Hz, 3H), 2.50-2.45 (m, 1H), 2.38 (s, 3H), 1.78-1.69 (m, 2H), 1.64-1.60 (m, 2H), 1.51-1.50 (m, 1H), 0.98-0.92 (m, 2H); 13C NMR (150 MHz, DMSO-d₆) δ 172.0, 168.9, 162.6, 161.9, 153.2, 148.0, 145.9, 137.6, 136.1, 131.8, 129.9, 125.1, 124.7, 122.7, 121.4, 119.9, 115.8, 50.9, 45.8, 41.7, 40.9, 33.0, 32.5, 32.3, 32.3, 32.0, 31.3, 26.0, 18.9; HRMS (ESI) m/z calcd for C₂₈H₃₀F₃N₅O₄ [M+H]+ 558.2323, found 558.2321.

### N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide (CDD-1146) (13)

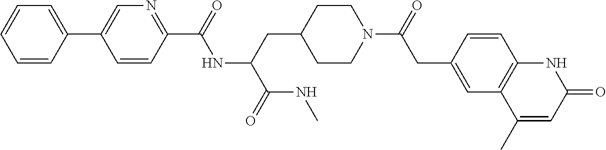 [SMILES: CNC(=O)C(CC1CCN(C(=O)Cc2ccc3[nH]c(=O)cc(C)c3c2)CC1)NC(=O)c1ccc(-c2ccccc2)cn1]

The title compound was prepared in a manner analogous to that which is described for 5-(2,4-dimethylphenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide (62% yield, white solid). ¹H NMR (600 MHz, DMSO-d₆) δ 11.56 (s, 1H), 8.98 (s, 1H), 8.66 (t, J=8.8 Hz, 1H), 8.29-8.27 (m, 1H), 8.12-8.10 (m, 2H), 7.8 (d, J=7.5 Hz, 2H), 7.55-7.53 (m, 3H), 7.49-7.46 (m, 1H), 7.34 (d, J=8.3 Hz, 1H), 7.24 (d, J=8.3 Hz, 1H), 6.38 (s, 1H), 4.60-4.58 (m, 1H), 4.36-4.32 (m, 1H), 3.98-3.93 (m, 1H), 3.74 (s, 2H), 2.97-2.88 (m, 1H), 2.61 (d, J=4.5 Hz, 3H), 2.5-2.46 (m, 1H), 2.38 (brs, 3H), 1.80 (brs, 1H), 1.73-1.52 (m, 4H), 1.02-0.92 (m, 2H); 13C NMR (150 MHz, DMSO-d₆) δ 172.2, 168.9, 163.5, 162.0, 148.6, 148.0, 147.0, 138.6, 137.6, 136.6, 136.0, 131.7, 131.8, 129.9, 129.7, 129.2, 127.6, 125.1, 122.5, 121.3, 119.9, 115.8, 50.7, 45.8, 41.7, 40.9, 33.0, 32.6, 32.3, 32.1, 31.4, 26.0, 18.9; HRMS (ESI) m/z calcd for C₃₃H₃₅N₅O₄ [M+H]+ 566.2762, found 566.2766. Chiral HPLC: Enantiomer I (50.285%, tᵣ=5.14 min); Enantiomer II (49.715%, tᵣ=9.29 min)

### tert-butyl 4-(2-(5-(2,4-dimethylphenyl)picolinamido)propyl)piperidine-1-carboxylate

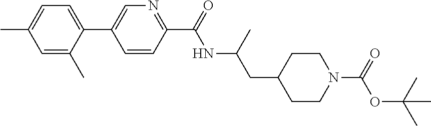 [SMILES: Cc1ccc(-c2ccc(C(=O)NC(C)CC3CCN(C(=O)OC(C)(C)C)CC3)nc2)c(C)c1]

To a solution of tert-butyl 5-(2,4-dimethylphenyl) picolinic acid (228 mg, 1 mmol, 1 equiv.), tert-butyl 4-(2-aminopropyl) piperidine-1-carboxylate (242 mg, 1 mmol, 1 equiv.), and HATU (570 mg, 1.5 mmol, 1.5 equiv.) in anhydrous DMF (5 mL) was added DIEA (175 μL, 1.5 mmol, 1.5 equiv.) under nitrogen. The reaction mixture was stirred at room temperature for 3 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to afford the title compound as a sticky solid (507 mg, 98% yield). ¹H NMR (600 MHz, DMSO-d₆) δ 8.58 (d, J=1.5 Hz, 1H), 8.51 (d, J=9.1 Hz, 1H), 8.08 (d, J=8.4 Hz, 1H), 7.95 (dd, J=8.0, 2.2 Hz, 1H), 7.17-7.16 (m, 2H), 7.13 (d, J=7.8 Hz, 1H), 4.21-4.16 (m, 1H), 3.89 (s, 2H), 2.33 (s, 3H), 2.22 (s, 3H), 1.77-1.75 (m, 1H), 1.67-1.62 (m, 1H), 1.59-1.57 (m, 1H), 1.48-1.42 (m, 1H), 1.37-1.31 (m, 11H), 1.17 (d, J=6.5 Hz, 3H), 1.04-1.57 (m, 2H); HRMS (ESI) m/z calcd for C₂₇H₃₇N₃O₃ [M+H]+ 452.2908, found 452.2912.

### 5-(2,4-dimethylphenyl)-N-(1-(1-(2-(4-methyl-2-oxo-1,2-dihydroquinolin-6-yl)acetyl)piperidin-4-yl)propan-2-yl)picolinamide (CDD-906) (14)

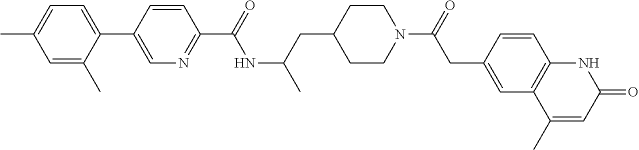 [SMILES: Cc1ccc(-c2ccc(C(=O)NC(C)CC3CCN(C(=O)Cc4ccc5[nH]c(=O)cc(C)c5c4)CC3)nc2)c(C)c1]

To a mixture of tert-butyl 4-(2-(5-(2,4-dimethylphenyl)picolinamido)propyl)piperidine-1-carboxylate (451 mg, 1 mmol, 1 equiv.), in 1,4-dioxane (20 mL) was added HCl/1,4-dioxane (4 M, 4 mL, 1.5 equiv.) dropwise via syringe at 20° C., and the resulting reaction mixture stirred for 1 h. After completion of the reaction all solvents were removed in vacuo and placed under high vacuum overnight. To the same vessel was added a mixture of DIEA (263 μL, 1.5 mmol, 1.5 equiv.), 2-(4-methyl-2-oxo-1,2-dihydroquinolin-6-yl) acetic acid (217 mg, 1 mmol, 1 equiv.), and HATU (570 mg, 1.5 mmol, 1.5 equiv.) in anhydrous DMF (10 mL) under nitrogen. The aqueous layer was extracted twice with EtOAc, and the combined organic extracts were washed with brine, dried over anhydrous Na₂SO₄, filtered and concentrated. The residue was purified by column chromatography on silica gel (CH₃OH/CH₂Cl₂, 1:99 to 5:95) to afford the title compound as a white solid (510 mg, 84% yield). ¹H NMR (600 MHz, DMSO-d₆) δ 11.57 (s, 1H), 8.55 (s, 1H), 8.51-8.49 (m, 1H), 8.08 (dd, J=7.9, 3.1 Hz, 1H), 7.9 (dd, J=7.9, 2.0 Hz, 1H), 7.5 (s, 1H), 7.33 (d, J=8.3 Hz, 1H), 7.24 (d, J=8.3 Hz, 1H), 7.13-7.12 (m, 2H), 7.08 (d, J=7.9 Hz, 1H), 6.37 (s, 1H), 4.37-4.33 (m, 1H), 4.18-4.17 (m, 1H), 3.96-3.92 (m, 1H), 3.73 (s, 2H), 3.39 (s, 3H), 2.95-2.85 (m, 1H), 2.49-2.43 (m, 1H), 2.37 (d, 3.1 Hz, 3H), 2.29 (s, 3H), 2.18 (s, 3H), 1.80-1.78 (m, 1H), 1.63-1.57 (m, 2H), 1.51-1.50 (m, 1H), 1.29-1.27 (m, 1H), 1.15 (d, J=6.3 Hz, 3H), 0.98-0.88 (m, 2H); 13C NMR (150 MHz, DMSO-d₆) δ 168.9, 164.4, 162.0, 150.7, 148.0, 146.4, 138.2, 137.6, 131.8, 130.8, 129.9, 129.6, 129.2, 128.5, 128.4, 125.1, 121.3, 119.9, 119.1, 115.8, 45.9, 41.8, 40.5, 39.6, 39.5, 36.9, 36.2, 33.4, 32.6, 31.9, 18.9 (2×); HRMS (ESI) m/z calcd for C₃₄H₃₈N₄O₃ [M+H]+ 551.3017, found 551.3004.

### tert-butyl (2-(1-(2-(4-methyl-2-oxo-1,2-dihydroquinolin-6-yl)acetyl)piperidin-4-yl)ethyl)carbamate

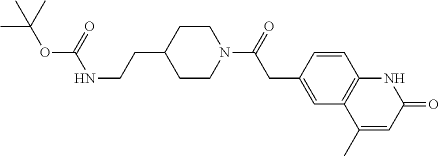 [SMILES: Cc1cc(=O)[nH]c2ccc(CC(=O)N3CCC(CCNC(=O)OC(C)(C)C)CC3)cc12]

To a solution of tert-butyl (2-(piperidin-4-yl) ethyl) carbamate (228 mg, 1 mmol, 1 equiv.), 2-(4-methyl-2-oxo-1,2-dihydroquinolin-6-yl) acetic acid (217 mg, 1 mmol, 1 equiv.), and HATU (570 mg, 1.5 mmol, 1.5 equiv.) in anhydrous DMF (5 mL) was added DIEA (175 μL, 1.5 mmol, 1.5 equiv.) under nitrogen. The reaction mixture was stirred at room temperature for 3 h and then quenched by the addition of water. The aqueous layer was extracted twice with EtOAc, and the combined organic extracts were washed with brine, dried over anhydrous Na₂SO₄, filtered and concentrated. The residue was purified by column chromatography on silica gel (CH₃OH/CH₂Cl₂, 1:99 to 5:95) to provide the title compound as a sticky oil (507 mg, 98% yield). ¹H NMR (600 MHz, CD₃OD) δ 7.65 (s, 1H), 7.43 (dd, J=8.4, 1.4 Hz, 1H), 7.29 (d, J=8.4 Hz, 1H), 6.49 (s, 1H), 4.54-4.51 (m, 1H), 4.05-4.03 (m, 1H), 3.89-3.83 (m, 2H), 3.07-3.03 (m, 3H), 2.66-2.61 (m, 1H), 2.49 (s, 3H), 1.75-1.69 (m, 2H), 1.59-1.54 (m, 1H), 1.41 (s, 9H), 1.36-1.34 (m, 2H), 1.07-0.90 (m, 2H); HRMS (ESI) m/z calcd for C₂₄H₃₃N₃O₄ [M+H]+ 428.2544, found 428.2534.

### tert-butyl (2-(4-(2-(4-methyl-2-oxo-1,2-dihydroquinolin-6-yl)acetyl)piperazin-1-yl)ethyl)carbamate

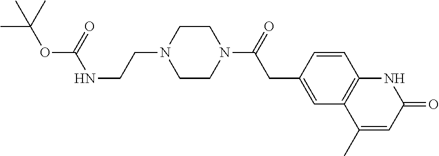 [SMILES: Cc1cc(=O)[nH]c2ccc(CC(=O)N3CCN(CCNC(=O)OC(C)(C)C)CC3)cc12]

The title compound was prepared in a manner analogous to that which is described for tert-butyl (2-(1-(2-(4-methyl-2-oxo-1,2-dihydroquinolin-6-yl)acetyl)piperidin-4-yl)ethyl)carbamate (92% yield, white solid). ¹H NMR (600 MHz, DMSO-d₆) δ 11.56 (s, 1H), 7.58-7.53 (m, 1H), 7.40-7.39 (m, 1H), 7.34 (dd, J=8.4, 1.5 Hz, 1H), 7.25 (t, J=8.4 Hz, 1H), 6.65 (s, 1H), 6.38 (s, 1H), 3.76 (s, 2H), 3.64 (s, 1H), 3.49-3.45 (m, 4H), 2.40-2.39 (m, 4H), 2.33-2.31 (m, 6H), 1.36 (s, 9H); HRMS (ESI) m/z calcd for C₂₃H₃₂N₄O₄ [M+H]+ 429.2496, found 429.2495.

### 5-(2,4-dimethylphenyl)-N-(2-(1-(2-(4-methyl-2-oxo-1,2-dihydroquinolin-6-yl)acetyl)piperidin-4-yl)ethyl)picolinamide (CDD-813) (15)

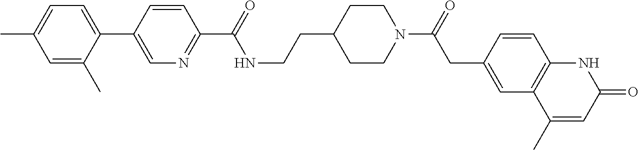 [SMILES: Cc1ccc(-c2ccc(C(=O)NCCC3CCN(C(=O)Cc4ccc5[nH]c(=O)cc(C)c5c4)CC3)nc2)c(C)c1]

Tert-butyl (2-(1-(2-(4-methyl-2-oxo-1,2-dihydroquinolin-6-yl)acetyl)piperidin-4-yl)ethyl)carbamate (536 mg, 1 mmol, 1 equiv.) was dissolved in DMF (3 mL) containing piperidine (10% v/v) and stirred 1 h in room temperature. Next, all solvents were removed in vacuo and the material was stored under high vacuum overnight. To the same vessel was added DIEA (439 μL, 2.5 mmol, 3 eq), 5-(2,4-dimethylphenyl) picolinic acid (140 mg, 0.7 mmol, 0.8 eq) and HATU (957.6 mg, 2.5 mmol, 3 equiv.) in anhydrous DMF (8 mL) under nitrogen. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to provide the title compound as a white solid (327 mg, 68% yield). ¹H NMR (600 MHz, DMSO-d₆) δ 11.56 (s, 1H), 8.82 (t, J=5.9 Hz, 1H), 8.57 (d, J=1.5 Hz, 1H), 8.07 (d, J=8.0 Hz, 1H), 8.07 (d, J=8.0 Hz, 1H), 7.94 (dd, J=7.9, 2.14 Hz, 1H), 7.54 (s, 1H), 7.35 (dd, J=7.9, 2.14 Hz, 1H), 7.25 (d, J=8.3 Hz, 1H), 7.17-7.15 (m, 2H), 7.11 (d, J=8.3 Hz, 1H), 6.38 (s, 1H), 4.39-4.39 (m, 1H), 3.37-3.33 (m, 2H), 3.98-3.96 (m, 1H), 3.76 (s, 2H), 2.96-2.94 (m, 1H), 2.54-2.50 (m, 1H), 2.39 (s, 3H), 2.32 (s, 3H), 2.21 (s, 3H), 1.73-1.70 (m, 2H), 1.55-1.45 (m, 3H), 0.97-0.91 (m, 2H); 13C NMR (150 MHz, DMSO-d₆) δ 148.9, 148.6, 148.0, 139.6, 138.3, 138.2, 137.6, 135.4, 134.6, 131.8, 131.7, 130.1, 131.7, 129.9, 127.3, 125.1, 121.8, 121.3, 119.9, 115.7, 54.0, 46.0, 41.9, 36.7, 36.2, 33.3, 32.6, 33.3, 32.6, 31.9, 21.1, 20.4, 18.9; HRMS (ESI) m/z calcd for C₃₃H₃₆N₄O₃ [M+H]+ 537.2860, found 537.2858.

### N-(2-(1-(2-(4-methyl-2-oxo-1,2-dihydroquinolin-6-yl)acetyl)piperidin-4-yl)ethyl)picolinamide (CDD-767) (16)

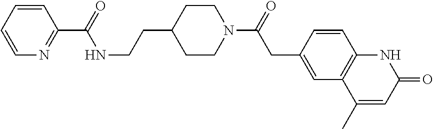 [SMILES: Cc1cc(=O)[nH]c2ccc(CC(=O)N3CCC(CCNC(=O)c4ccccn4)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (80% yield, white solid). ¹H NMR (600 MHz, DMSO-d₆) δ 11.55 (s, 1H), 8.77 (t, J=5.9 Hz, 1H), 8.62 (d, J=2.1 Hz, 1H), 8.05-8.02 (m, 1H), 7.99-7.96 (m, 1H), 7.59-7.57 (m, 1H), 7.53 (s, 1H), 7.34 (dd, J=8.4, 1.8 Hz, 1H), 7.24 (d, J=8.3 Hz, 1H), 6.38 (s, 1H), 4.37-4.35 (m, 1H), 3.97-3.95 (m, 1H), 3.75 (s, 2H), 3.343.31 (m, 2H), 2.97-2.93 (m, 1H), 2.54-2.50 (m, 1H), 2.38 (s, 3H), 1.70-1.69 (m, 2H), 1.53-1.43 (m, 3H), 0.97-0.89 (m, 2H); 13C NMR (150 MHz, DMSO-d₆) δ 168.5, 163.7, 161.5, 150.1, 148.3, 147.6, 137.7, 137.2, 131.4, 129.4, 126.3, 124.7, 121.8, 120.9, 119.4, 115.3, 45.5, 41.4, 39.0, 36.2, 35.7, 32.9, 32.1, 31.4, 18.4; HRMS (ESI) m/z calcd for C₂₅H₂₈N₄O₃ [M+H]+ 433.2234, found 433.2225.

### N-(2-(1-(2-(4-methyl-2-oxo-1,2-dihydroquinolin-6-yl)acetyl)piperidin-4-yl)ethyl)benzamide (CDD-779) (17)

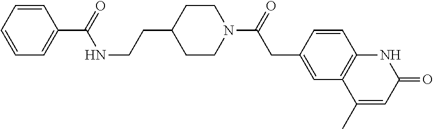 [SMILES: Cc1cc(=O)[nH]c2ccc(CC(=O)N3CCC(CCNC(=O)c4ccccc4)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (80% yield, white solid). ¹H NMR (600 MHz, DMSO-d₆) δ 11.55 (s, 1H), 8.41 (t, J=5.4 Hz, 1H), 7.82 (d, J=4.2 Hz, 1H), 7.54 (s, 1H), 7.52-7.49 (m, 1H), 7.47-7.43 (m, 2H), 7.35 (dd, J=8.4, 1.5 Hz, 1H), 7.24 (d, J=8.3 Hz, 1H), 6.38 (s, 1H), 4.39-4.37 (m, 1H), 3.98-3.96 (m, 1H), 3.76 (s, 2H), 3.30-3.27 (m, 2H), 2.98-2.94 (m, 1H), 2.55-2.50 (m, 1H), 2.39 (s, 3H), 1.70 (t, J=11.4 Hz, 2H), 1.56-1.51 (m, 1H), 1.45-1.42 (m, 2H), 0.98-0.90 (m, 2H); 13C NMR (150 MHz, DMSO-d₆) δ 168.5, 166.0, 161.5, 150.5, 147.5, 139.6, 137.2, 134.6, 131.3, 130.9, 128.5, 127.0, 124.7, 120.9, 120.3, 119.4, 115.3, 45.5, 41.4, 39.0, 36.6, 35.6, 32.9, 32.2, 31.4, 18.4; HRMS (ESI) m/z calcd for C₂₆H₂₉N₃O₃ [M+H]+ 432.2282, found 432.2275.

### N-(2-(1-(2-(4-methyl-2-oxo-1,2-dihydroquinolin-6-yl)acetyl)piperidin-4-yl)ethyl)quinoline-2-carboxamide (CDD-855) (18)

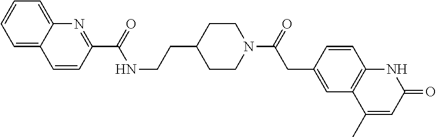 [SMILES: Cc1cc(=O)[nH]c2ccc(CC(=O)N3CCC(CCNC(=O)c4ccc5ccccc5n4)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (80% yield, white solid). ¹H NMR (600 MHz, DMSO-d₆) δ 11.58 (s, 1H), 8.92 (t, J=6.0 Hz, 1H), 8.52 (d, J=8.4 Hz, 1H), 8.15 (d, J=8.4 Hz, 1H), 8.12 (d, J=8.4 Hz, 1H), 8.04 (d, J=8.0 Hz, 1H), 7.85-7.82 (m, 1H), 7.70-7.67 (m, 1H), 7.52 (s, 1H), 7.34 (dd, J=8.9, 2.0 Hz, 1H), 7.25 (d, J=8.4 Hz, 1H), 6.38 (s, 1H), 4.38-4.36 (m, 1H), 3.96-3.94 (m, 1H), 3.75 (s, 2H), 3.41-3.37 (m, 2H), 2.96-2.92 (m, 1H), 2.53-2.49 (m, 1H), 2.37 (s, 3H), 1.71-1.69 (m, 1H), 1.54-1.47 (m, 3H), 0.98-0.91 (m, 2H); 13C NMR (150 MHz, DMSO-d₆) δ 168.9, 162.9, 161.5, 148.4, 148.0, 147.5, 139.2, 137.8, 137.7, 137.2, 134.9, 134.1, 131.2, 129.6, 129.4, 126.8, 124.6, 121.4, 119.4, 115.3, 45.5, 41.8, 41.4, 32.4, 31.8, 31.2, 20.6, 19.8, 18.4; HRMS (ESI) m/z calcd for C₂₉H₃₀N₄O₃ [M+H]+ 483.2391, found 483.2382.

### N-(2-(1-(2-(4-methyl-2-oxo-1,2-dihydroquinolin-6-yl)acetyl)piperidin-4-yl)ethyl)isoquinoline-3-carboxamide (CDD-854) (19)

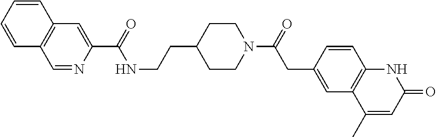 [SMILES: Cc1cc(=O)[nH]c2ccc(CC(=O)N3CCC(CCNC(=O)c4cc5ccccc5cn4)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (81% yield, white solid). ¹H NMR (600 MHz, DMSO-d₆) δ 11.54 (s, 1H), 9.37 (s, 1H), 8.91 (t, J=5.9 Hz, 1H), 8.54 (s, 1H), 8.24 (d, J=8.0 Hz, 1H), 8.15 (d, J=8.1 Hz, 1H), 7.87 (t, J=4.0 Hz, 1H), 7.80 (t, J=4.0 Hz, 1H), 7.54 (s, 1H), 7.35 (dd, J=8.4, 1.5 Hz, 1H), 7.24 (d, J=8.3 Hz, 1H), 6.38 (s, 1H), 4.38-4.36 (m, 1H), 3.99-3.96 (m, 1H), 3.76 (s, 2H), 3.41-3.37 (m, 2H), 2.99-2.95 (m, 1H), 2.56-2.50 (m, 1H), 2.39 (s, 3H), 1.74 (s, 2H), 1.57-1.47 (m, 3H), 0.98-0.94 (m, 2H); 13C NMR (150 MHz, DMSO-d₆) δ 168.4, 163.9, 161.4, 151.4, 147.5, 143.9, 137.1, 135.3, 131.3, 131.2, 129.4, 129.1, 128.9, 129.1, 128.9, 127.9, 127.7, 124.6, 120.8, 119.5, 119.3, 115.2, 45.5, 41.8, 41.4, 35.8, 32.9, 32.1, 18.4, 18.0, 16.7; HRMS (ESI) m/z calcd for C₂₉H₃₀N₄O₃ [M+H]+ 483.2391, found 483.2387.

### N-(2-(1-(2-(4-methyl-2-oxo-1,2-dihydroquinolin-6-yl)acetyl)piperidin-4-yl)ethyl)isoquinoline-1-carboxamide (CDD-853) (20)

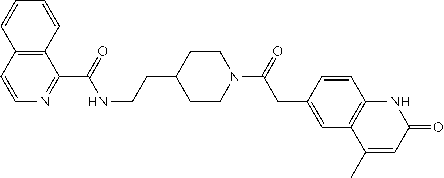 [SMILES: Cc1cc(=O)[nH]c2ccc(CC(=O)N3CCC(CCNC(=O)c4nccc5ccccc45)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (84% yield, white solid). ¹H NMR (600 MHz, DMSO-d₆) δ 11.57 (s, 1H), 8.91 (d, J=8.5 Hz, 1H), 8.83 (t, J=5.8 Hz, 1H), 8.52 (d, J=5.5 Hz, 1H), 8.02 (d, J=8.2 Hz, 1H), 7.98 (d, J=5.5 Hz, 1H), 7.81-7.79 (m, 1H), 7.72-7.70 (m, 1H), 7.54 (s, 1H), 7.35 (dd, J=8.4, 1.5 Hz, 1H), 7.25 (d, J=8.3 Hz, 1H), 6.38 (s, 1H), 4.40-4.38 (m, 1H), 3.99-3.97 (m, 1H), 3.76 (s, 2H), 3.40-3.37 (m, 2H), 2.98-2.94 (m, 1H), 2.55-2.50 (m, 1H), 2.38 (s, 3H), 1.74-1.72 (m, 2H), 1.59-1.55 (m, 1H), 1.51-1.47 (m, 2H), 1.00-0.93 (m, 2H); 13C NMR (150 MHz, DMSO-d₆) δ 168.5, 166.0, 161.5, 151.4, 147.5, 140.7, 137.1, 136.5, 131.3, 130.5, 129.4, 128.2, 127.0, 126.5, 125.4, 124.6, 123.1, 120.8, 119.4, 115.3, 45.5, 41.8, 41.4, 36.3, 35.6, 32.9, 32.1, 18.4, 18.0, 16.6; HRMS (ESI) m/z calcd for C₂₃H₃₀N₄O₃ [M+H]+ 483.2391, found 483.2389.

### N-(2-(4-(2-(4-methyl-2-oxo-1,2-dihydroquinolin-6-yl)acetyl)piperazin-1-yl)ethyl)picolinamide (CDD-778) (21)

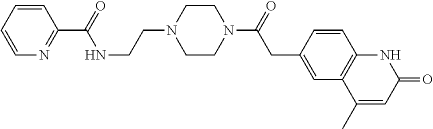 [SMILES: Cc1cc(=O)[nH]c2ccc(CC(=O)N3CCN(CCNC(=O)c4ccccn4)CC3)cc12]

The title compound was prepared in a manner analogous to that which is described for 5-(2,4-dimethylphenyl)-N-(2-(1-(2-(4-methyl-2-oxo-1,2-dihydroquinolin-6-yl)acetyl)piperidin-4-yl)ethyl)picolinamide (81% yield, white solid). ¹H NMR (600 MHz, CD₃OD) δ 8.63 (d, J=4.4 Hz, 1H), 8.11 (d, J=7.8 Hz, 1H), 7.99-7.96 (m, 1H), 7.68 (s, 1H), 7.58-7.96 (m, 1H), 7.46 (dd, J=8.4, 1.6 Hz, 1H), 7.33 (d, J=8.4 Hz, 1H), 6.52 (s, 1H), 3.95 (s, 2H), 8.63 (d, J=4.4 Hz, 1H), 3.73-3.35 (m, 6H), 3.31-3.30 (m, 4H), 2.51 (s, 3H); 13C NMR (150 MHz, DMSO-d₆) δ 172.1, 168.1, 164.8, 151.5, 150.3, 149.8, 138.9, 138.3, 133.1, 130.7, 128.1, 126.4, 123.3, 121.9, 120.9, 117.4, 58.0, 53.09 (2×), 43.8, 39.9, 35.2, 19.08 (2×); HRMS (ESI) m/z calcd for C₂₄H₂₇N₅O₄ [M+H]+ 434.2187, found 434.2186.

### Example 3: Mosher's Acid Stereochemical Analysis

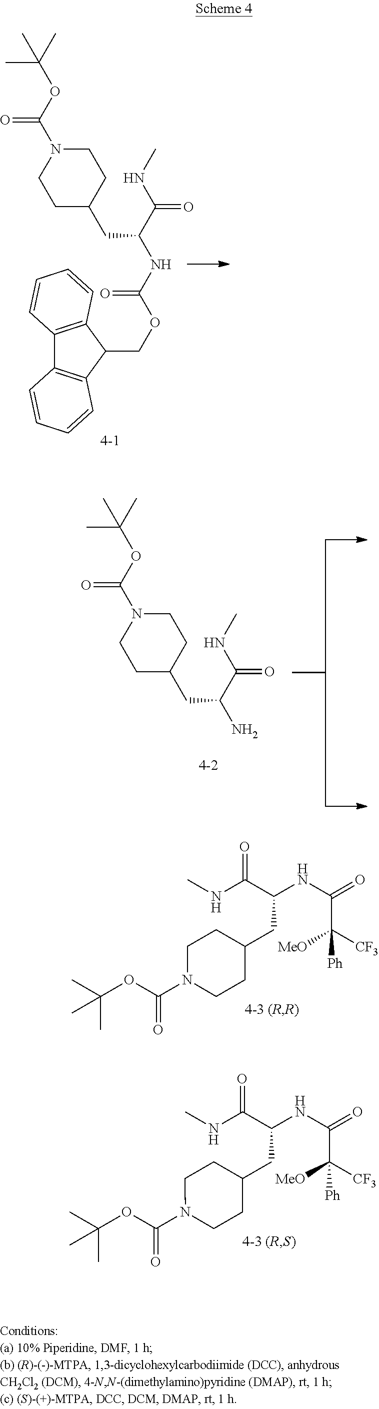

Mosher's method was utilized to determine the absolute stereochemistry of α-chiral carbon of 4-1 (Scheme 4), which has been extrapolated to define the stereochemistry of the compounds of the present disclosure. First, the FMOC protecting group was removed from 4-1 to provide 4-2. Compound 4-2 was subsequently coupled with both enantiomers of α-methoxy-α-trifluoromethylphenylacetic acid (MTPA), independently, to provide the MTPA adducts 4-3 (R,R) (R-MTPA amide) and 4-3 (R,S) (S-MTPA anide).

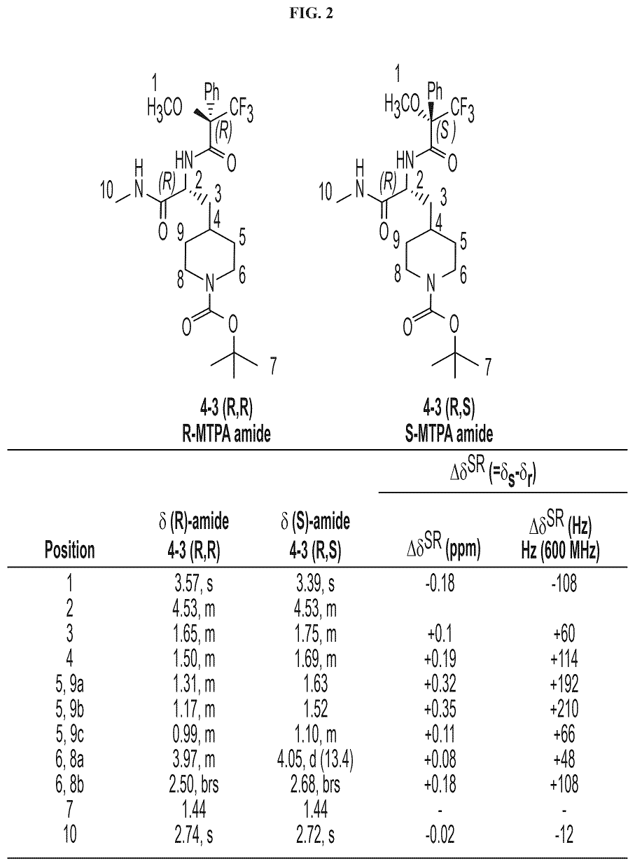

Next, the compounds were subjected to chiral analysis by determining ΔδSR (ppm). A clear difference was observed in the chemical shift at positions 3, 4, 5 and 9, which are affected by shielding effect of phenyl group of MTPA. Detailed chemical shifts, ΔδSR (ppm) and ΔδSR (Hz) are depicted in FIG. 2. Protons on position 3 and 4, adjacent to the α-carbon have shown positive values of ΔδSR (ppm) and ΔδSR (Hz), which indicates below the plane. Whereas protons on position 10 shows above the plane and shown negative values of ΔδSR (ppm) and ΔδSR (Hz). Detailed H1 NMR spectra of MTPA amides are mentioned in supporting data. From this experiment it is clear that compound 4-1 comprises a stereocenter having the (R)-configuration. Thus, compounds 2 (CDD-787) and 4 (CDD-956) also comprise a stereocenter having the (R)-configuration, which is in agreement with the observed positive optical rotation.

### Example 4: Validation of BD1 DEC-Tec Selection Hits

Candidate hit molecules were synthesized by truncating the DNA barcode linkage down to a methyl amide. The prioritization of compounds to synthesize off-DNA for hit-confirmation was based on sequence count and structural features that were common among the enriched sequences. Accordingly, the 3-cycle library hit compound 2 (CDD-787) (138 counts), its enantiomer, compound 4 (CDD-786), and racemic compound 1 (CDD-724) (267 counts), were selected for initial synthesis. The synthetic route and detailed experimental of the DEC-Tec hits is described elsewhere herein.

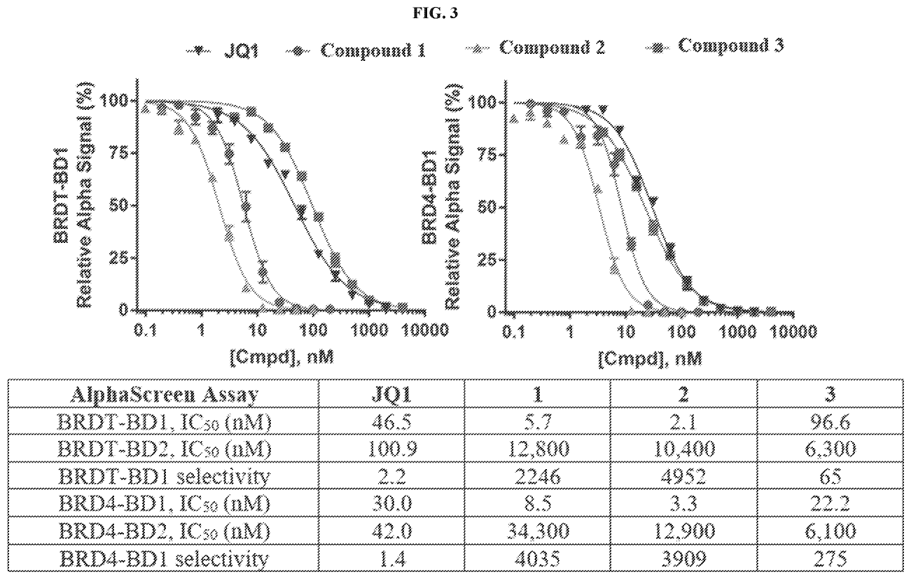

Compound 1 and each enantiomer thereof (i.e., compounds 2 and 3) were functionally screened for BRDT-BD1 activity using an AlphaScreen competition assay with biotinylated JQ1 as the ligand. All three compounds demonstrated very potent BRDT-BD1 inhibitory activity with nanomolar IC₅₀ values (FIG. 3). These compounds were improved in their selectivity of BRDT-BD1 over the pan-BET inhibitor (+)-JQ1. They also had significantly less affinity for BRDT-BD2 with IC₅₀ values of 6.3 μM-12.8 μM or ˜65-4952-fold lower activity, compared to the 2-fold BD1 selectivity observed with JQ1 (FIG. 3). Using a similar AlphaScreen assay, the BRD4 activity of the compounds was also tested and a similar BD1 preference was observed (FIG. 3). It was noted that compound 3 has 4-fold greater affinity for BRD4-BD1 than BRDT-BD1. From the initial AlphaScreen assay, compound 2, having the (R)-configuration, emerged as the most selective BRDT-BD1 inhibitor with single digit nanomolar activity (IC₅₀ 2.1 nM) and ˜5000-fold selectivity over BRDT-BD2. Thus, compound 2 is 22-fold more potent than, and ˜2200 fold more selective than, (+)-JQ1 for BRDT-BD1. The (S)-enantiomer (i.e., compound 3), and the racemate (i.e., compound 1), gave IC₅₀ values of 96.6 nM and 5.7 nM, respectively to BRDT-BD1 and displayed 65-fold and 2246-fold selectivity, respectively, over BRDT-BD2.

### Example 5: Exploration of Structure-Activity Relationships (SAR)

Based on the promising inhibition of the compounds that were most enriched by the selection, SAR around the pyridine and 1,2-dihydroquinolinone rings was pursued. The goal was to identify compounds that struck an optimal balance between potency and selectivity to BET BD1 over BET BD2 domains. First, it was explored whether the 3,5-dimethyl phenyl substituent of the pyridine ring could be replaced with smaller groups such as a trifluoromethyl or a simple phenyl ring. Removal of the methyl groups on the phenyl ring of the (R) isomer provided compound 4, which exhibited enhanced potency (IC₅₀ 2.1 nM and 4.4 nM to BRDT-BD1 and BRD4-BD1, respectively). However, the similar deletion of the methyl groups of the (S)-isomer (i.e., compound 5, CDD-2107) and racemate (i.e., compound 13, CDD-1146), resulted in 3.6-fold and 21-fold drop in potency to BRDT BD1 compared to parent compound 2.

An improvement in potency was observed when the phenyl moiety of the 3 was replaced with a trifluoromethyl group to yield compound 7 (CDD-819), having IC₅₀ 3.8 nM and 7.4 nM to BRDT-BD1 and BRD4-BD1, respectively. However, the selectivity over BRD4-BD2 was reduced.

Interestingly, for the (R)-isomeric series, monohalogenation (i.e., compound 8, CDD-981) and dihalogenation (i.e., compound 9, CDD-982) on the phenyl ring maintained excellent potency to BRDT-BD1 (IC₅₀ 5.1 nM and 4.7 nM respectively) but their BRDT-BD1: BRDT-BD2 selectivity was decreased compared to compound 2. Compound 11 (CDD-784), which contained no phenyl substitution on the pyridine ring, sustained BD1 potency (BRDT-BD1 IC₅₀ 20.2 nM and BRD4-BD1 15.7 nM), but underwent a 5-fold loss of selectivity to BRDT-BD2 compared to compound 2.

TABLE 2
Activities of Compounds 4-5 and 8-9

| | BRDT-BD1 | BRDT-BD2 | BRDT-BD1 | BRD4-BD1 | BRD4-BD2 | BRD4-BD1 |
|---|---|---|---|---|---|---|
| | IC₅₀ | IC₅₀ | vs. BRDT-BD2 | IC₅₀ | IC₅₀ | vs. BRD4-BD2 |
| Cmpd | (nM) | (nM) | fold | (nM) | (nM) | fold |
| 4 | 2.1 | 12,895 | 6,140 | 4.4 | 2,640 | 600 |
| 5 | 45.4 | 5,066 | 111 | 41.1 | 7,158 | 174 |
| 8 | 5.1 | 33,832 | 6,633 | 12.8 | 19,883 | 1,553 |
| 9 | 4.7 | 22,559 | 4,799 | 9.8 | 13,951 | 1,423 |

Next, focus of the SAR studies was shifted to the 1,2-dihydroquinoline moiety. Removal of the methyl group on 1,2-dihydroquinolinone (i.e., compound 10, CDD-986) provided single digit nanomolar potency for both BRDT-BD1 and BRD4-BD1 but gave a 1.6-fold loss of selectivity over BRD4-BD2 (Table 3).

TABLE 3
Activities of Compounds 10-13

| Assay | 10 | 11 | 12 | 13 |
|---|---|---|---|---|
| BRDT-BD1 IC₅₀ (nM) | 5.4 | 20.2 | 14.0 | 7.6 |
| BRDT-BD2 IC₅₀ (nM) | 20,930 | 19,400 | 7,186 | —^{a} |
| BRDT-BD1 vs. BRDT-BD2 fold | 3,875 | 960 | 513 | —^{a} |
| BRD4-BD1 IC₅₀ (nM) | 7.1 | 15.7 | 6.9 | —^{a} |
| BRD4-BD2 IC₅₀ (nM) | 16,701 | 32,640 | 8,800 | —^{a} |
| BRD4-BD1 vs. BRD4-BD2 fold | 2,352 | 2,078 | 1,275 | —^{a} |

^{a}not performed.

A second round of SAR was conducted in which focus was placed on focused racemates, truncation of the amide on propenamide linker, and the replacement of 5-substituted pyridines with diverse quinolines (Table 4).

TABLE 4
Activities of Compounds 14-21

| | BRDT-BD1 | BRDT-BD2 | BRDT-BD1 | BRD4-BD1 | BRD4-BD2 | BRD4-BD1 |
|---|---|---|---|---|---|---|
| | IC₅₀ | IC₅₀ | vs. BRDT-BD2 | IC₅₀ | IC₅₀ | vs. BRD4-BD2 |
| Cmpd | (nM) | (nM) | fold | (nM) | (nM) | fold |
| 14 | 31 | 23,449 | 756 | 52.4 | 20,000 | 381 |
| 15 | 33.7 | 67,836 | 2012 | 27.8 | 8,689 | 312 |
| 16 | 35.4 | 6,800 | 192 | 13.8 | 75,200 | 5,449 |
| 17 | 148.2 | 10,400 | 70 | 47.9 | —^{a} | —^{a} |
| 18 | 81.3 | —^{a} | —^{a} | 122.4 | —^{a} | —^{a} |
| 19 | 115.7 | —^{a} | —^{a} | 119.4 | —^{a} | —^{a} |
| 20 | 638.1 | —^{a} | —^{a} | 341.4 | —^{a} | —^{a} |
| 21 | 5,200 | 71,800 | 13.8 | 1300 | —^{a} | —^{a} |

^{a}not efficient

In case of compound 14 (CDD-906), replacing methyl instead of amide on linker exhibited reduced potency (IC₅₀ 31 nM) and 6.5-fold loss of selectivity on BRDT-BD1. Further modifications on phenyl pyridines such as observed in compounds 15-17 (CDD-813, CDD-767 and CDD-779), containing truncated amides demonstrates dramatic drop of activities.

Introduction of quinoline (i.e., compound 18, CDD-855), iso-quinolines (i.e., compounds 19-20, CDD-854 and CDD-853), and piperazine (i.e., compound 21, CDD-778) moieties did not improve either selectivity or potency. From this it is noted that both amide and phenyl pyridines functionalities may be considered important for maintaining activity. Overall, it was concluded from these SAR studies that compounds in the (R)-isomeric series, such as compounds 4 and 8-10), gave excellent BRDT-BD1 potency with superior selectivity over BRDT-BD2.

### Example 6: Metabolic Stability Studies

The metabolic stabilities of compounds 2 and 4 in mouse and human liver microsomes was determined. In mouse liver microsomes, compound 2 is labile, like JQ1, while compound 4 is relatively more stable. In human liver microsomes, the half-life of compound 4 is 139 minutes, while compound 2 is 33 minutes, and JQ1 is quite low (Table 5).

TABLE 5
Metabolic stability of compound 2 and 4 in mouse liver
microsomes (MLM) and human liver microsomes (HLM)

| Entry | Assay | 2 | 4 | JQ1 | Alprazolam |
|---|---|---|---|---|---|
| 1 | MLM t_{1/2} (min) | 13 | 35 | 13 | 302 |
| 2 | HLM t_{1/2} (min) | 33 | 139 | 10.3 | 864 |

Final concentrations: liver microsomes: 0.5 mg protein/mL; compound concentration: 2.0 μM; NADPH concentration: 1.0 mM; JQ1: short half-life control; Alprazolam: long half-life control. In duplicate at 0, 5, 10, 20, 40, and 60 min. Half-lives less than 30 min in MLM and HLM was generally considered unstable.

### Example 7: Picomolar Activity of BET BD1 Inhibitors

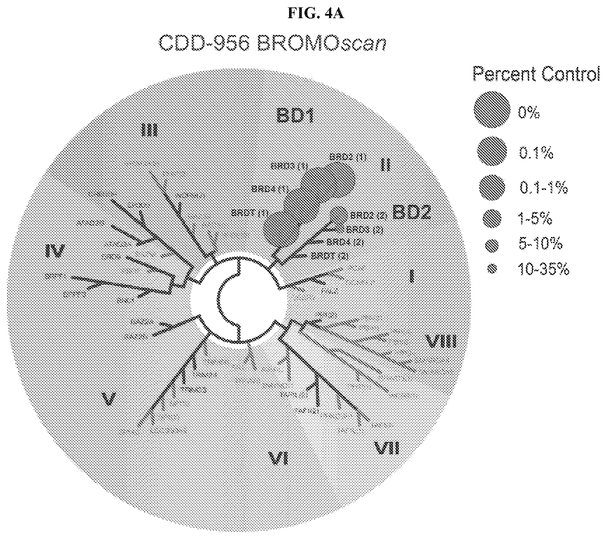

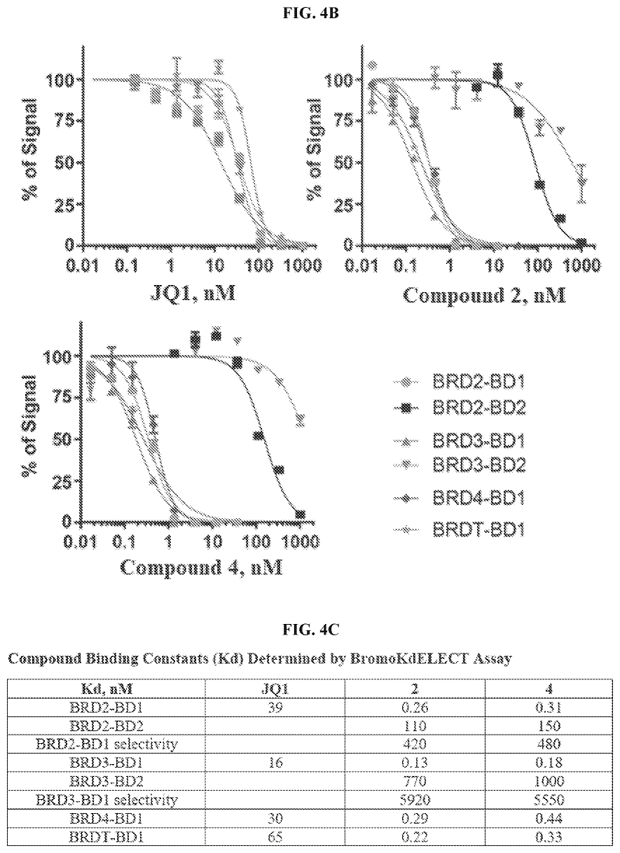

To assess the selectivity of compound 4, a BROMOscan was performed, which resulted in identification of high selectivity for the BET family, and primarily BD1 of all BET family members (FIG. 4A). Compound 4 also had some binding to BD2 of BRD2 and BRD3, while exhibiting no binding in the BROMOscan to the BRD4-BD2 nor BRDT-BD2. Next, the binding affinity was compared in the BromoKdELECT assay (FIGS. 4B-4C). Both of compounds 2 and 4 exhibited picomolar inhibition against BRD2-BDT1 (IC₅₀ 260 pM and 310 pM), BRD3-BD1 (IC₅₀ 130 pM and 180 pM), BRD4-BD1 (IC₅₀ 290 pM and 440 pM) and BRDT-BD1 (IC₅₀ 220 pM and 330 pM), respectively; which is outstanding compared with reference JQ1 which has an IC₅₀ ranging from about 39 nM to about 65 nM. In the case of the BRD2-BD1 selectivity, both compounds 2 and 4 showed superior selectivity (420-fold for compound 2 and 480-fold for compound 4) over BRD2-BD2. Whereas in case of BRD3-BD1 selectivity, compound 2 exhibited more selectivity than compound 4, and showed 5920-fold selectivity over BRD3-BD2.

TABLE 6
Matrix of BROMOscan assay screen of CDD-956 (compound 4)^{a}

| | | | Compound |
|---|---|---|---|
| DiscoveRx Gene | Entrez Gene | Percent | Concentration |
| Symbol | Symbol | Control | (nM) |
| ATAD2A | ATAD2 | 100 | 1000 |
| ATAD2B | ATAD2B | 97 | 1000 |
| BAZ2A | BAZ2A | 100 | 1000 |
| BAZ2B | BAZ2B | 100 | 1000 |
| BRD1 | BRD1 | 96 | 1000 |
| BRD2(1) | BRD2 | 0 | 1000 |
| BRD2(2) | BRD2 | 2 | 1000 |
| BRD3(1) | BRD3 | 0 | 1000 |
| BRD3(2) | BRD3 | 26 | 1000 |
| BRD4(1) | BRD4 | 0 | 1000 |
| BRD4(2) | BRD4 | 78 | 1000 |
| BRD7 | BRD7 | 95 | 1000 |
| BRD9 | BRD9 | 90 | 1000 |
| BRDT(1) | BRDT | 0 | 1000 |
| BRDT(2) | BRDT | 68 | 1000 |
| BRPF1 | BRPF1 | 66 | 1000 |
| BRPF3 | BRPF3 | 100 | 1000 |
| CECR2 | CECR2 | 100 | 1000 |
| CREBBP | CREBBP | 100 | 1000 |
| EP300 | EP300 | 100 | 1000 |
| FALZ | BPTF | 100 | 1000 |
| GCN5L2 | KAT2A | 99 | 1000 |
| PBRM1(2) | PBRM1 | 100 | 1000 |
| PBRM1(5) | PBRM1 | 100 | 1000 |
| PCAF | KAT2B | 100 | 1000 |
| SMARCA2 | SMARCA2 | 100 | 1000 |
| SMARCA4 | SMARCA4 | 100 | 1000 |
| TAF1(2) | TAF1 | 88 | 1000 |
| TAF1L(2) | TAF1L | 100 | 1000 |
| TRIM24(PHD, Bromo.) | TRIM24 | 100 | 1000 |
| TRIM33(PHD, Bromo.) | TRIM33 | 84 | 1000 |
| WDR9(2) | BRWD1 | 100 | 1000 |

^{a}Percent control (% Ctrl): Compound 4 was screened at 1000 nM and results for binding interactions are reported as “% Ctrl”, wherein lower numbers indicate stronger hits in the matrix; % Ctrl Calculation: (4 signal-positive control signal)/(negative control signal-positive control signal)*100; negative control is DMS (100% Ctrl); positive control is control compound (0% Ctrl).

### Example 8: Crystal Structure Validates Importance of R Stereochemistry in BD1 Selectivity

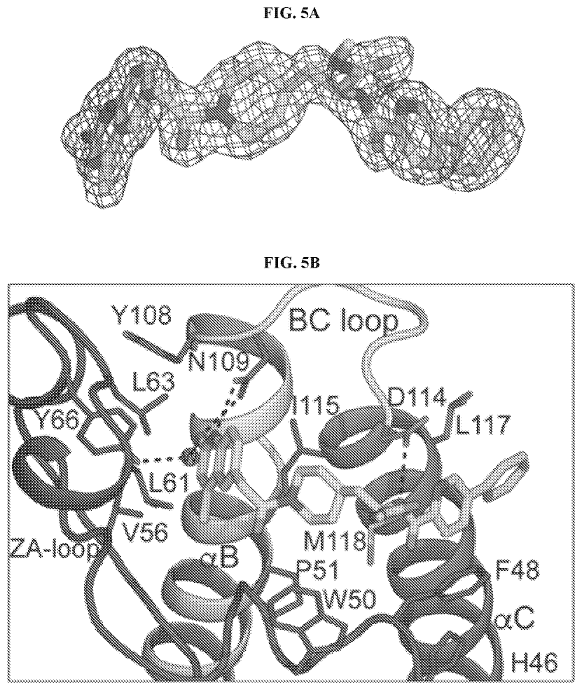

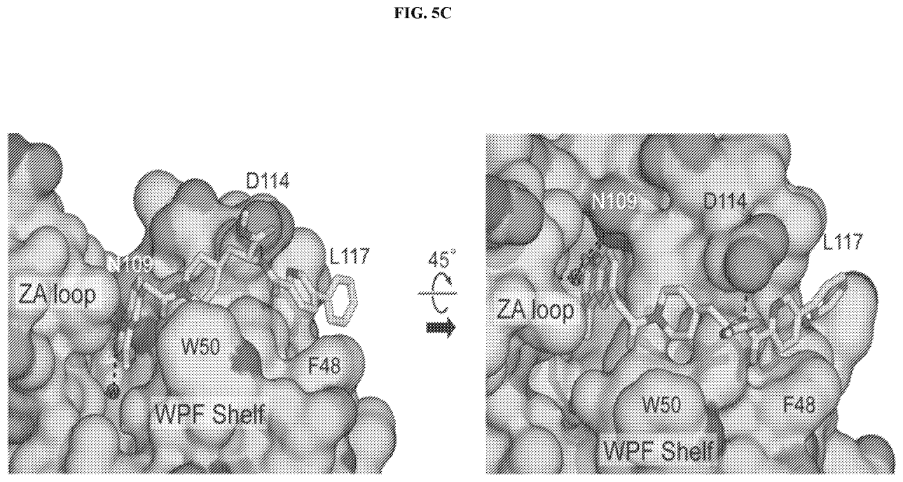

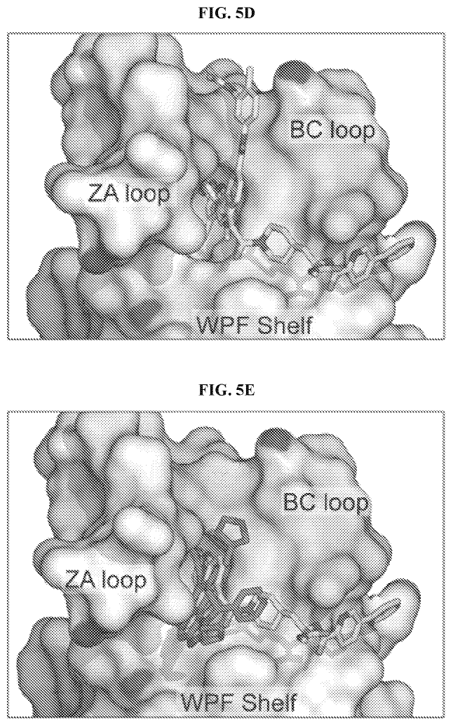

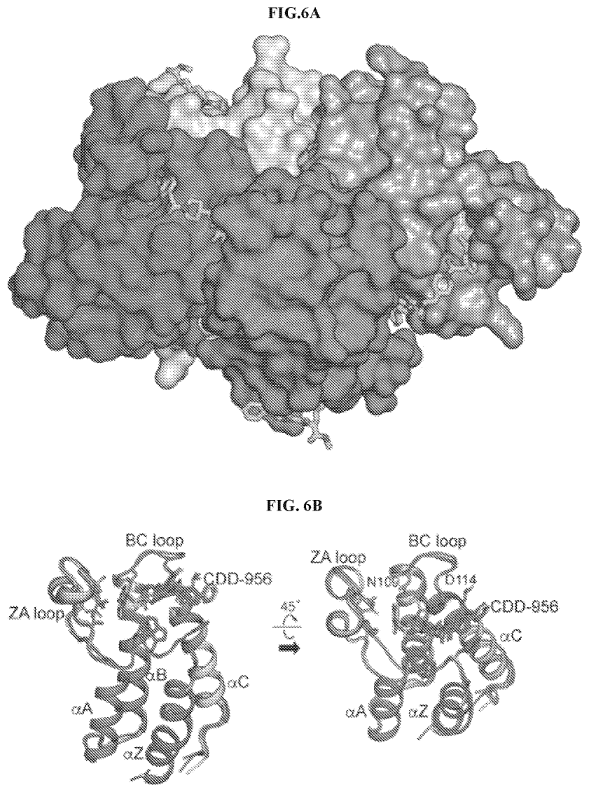

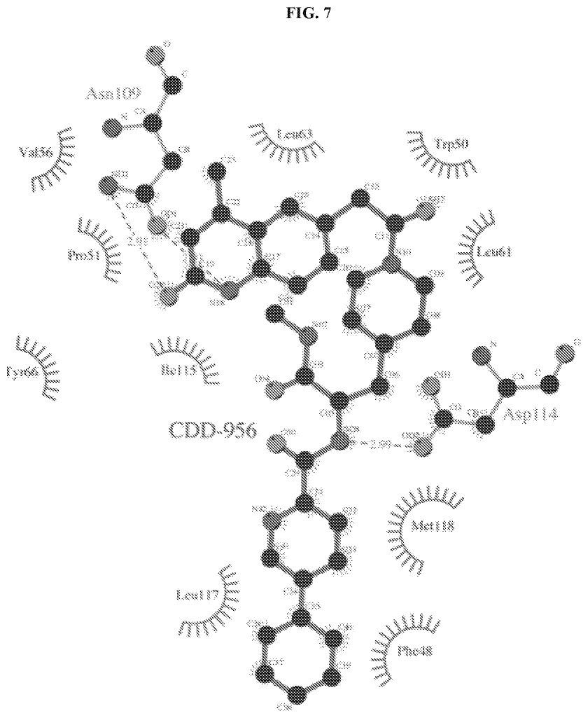

To understand the basis for the high affinity BET BD1 interaction and selectivity, the crystal structure of BRDT-BD1 with compound 4 was determined at 1.82 Å resolution (FIG. 5A/Table 7); Protein Data Bank (PDB) ID: 7UBO). The BRDT-BD1/CDD-956 (compound 4) crystal contained four molecules per asymmetric unit (FIG. 6A) that are very similar, showing RMSD values of >0.5 Å between shared ˜100 CA atoms (FIG. 6B). These four domains all show very similar poses of the compound 4 ligand contacting the KAc binding pocket and a neighboring hydrophobic groove. Although all four compound 4 molecules participate in crystal packing interactions, the binding poses of compound 4 are nearly identical, suggesting that these contacts minimally influence the binding mode.

Compound 4 binds to the KAc binding pocket, the WPF shelf, and a shallow hydrophobic groove formed between αZ and αC helices (FIGS. 5A-5B). The methyl-quinoline binds to the KAc pocket via hydrogen bonds directly with the conserved asparagine (N109) at the B-C loop and indirectly with Y66 at the ZA loop through ordered water. The methyl-quinoline interacts with hydrophobic residues F52, V56, L61, and L63 that line the outer rim of the KAc pocket. Several ordered waters are present in the pocket, including one that mediates the Y66: methyl-quinoline interaction. The piperidine-amide linker interacts with the WPF shelf and the αC helix, and the amide forms a hydrogen bond with D114 at the beginning of the αC helix (FIG. 5B). The phenyl-pyridine docks to a shallow hydrophobic groove between the αZ and αC helices. Side chains of F48 (αZ), D114 (αC), LI 17(αC), and M118 (αC) form the hydrophobic groove (FIG. 5C). The pyridine orients perpendicular to the side chains of F48 and D114 on either side and interacts with these residues and the side chain of M118 through van der Waals (vdW) contacts. The plane of the phenyl ring is twisted 38 degrees relative to the pyridine ring and packs against the side chain of L117(αC) (FIG. 5C). Comparison of the binding modes of compound 4 with BDT, CDD-1302 with BRDT-BD2, and iBET-BD1 (GSK778) with BRD4-BD1 (FIGS. 5D-5E) shows that the BD1-selective and BD2-selective DECL-derived inhibitors each occupy the same KAc pocket as GSK778 but also access adjacent grooves that differ between the two domain types.

Structural comparisons between this structure and previous BD domains explain the high BD1:BD2 selectivity seen for compound 4 based on three key residues that differ between the domains: F48, L117, and D114. Phenylalanine 48 (αZ) is replaced with tyrosine in all BD2s (Y291 in BRDT). The hydrophobic groove to which compound 4 binds is missing in BRDT BD2 because the side chain of Y291 points away from the BC loop and hydrogen bonds with H287 located one helix turn below. L117(αC) is replaced in BD2s with threonine (BRDT) or alanine (BRD2, BRD3, and BRD4) reducing the vdW contact surface with the phenyl group of compound 4. Aspartate 114, which packs with and forms hydrogen bonds to the amide linker, is replaced with glutamate in all BD2s except BRD2. While other BD2s cannot form the critical hydrogen bond with the amide linker seen in the crystal structure, and may clash with the phenyl-pyridine, BRD2-BD2 can presumably make similar interactions to those seen in the complex described herein. The presence of this aspartate may explain why BRD2-BD2 binds compound 4 tighter than BRD3-BD2 (FIG. 5C).

In one aspect, the crystal structure provides an explanation as to the potency of the DECL library hit and the relative affinities of the R and S enantiomers of the initial hit and subsequent analogues. The methyl groups of compound 2 (i.e., R-enantiomer of the original hit compound 1), can be accommodated in the structure without steric clash. Without wishing to be bound by theory, intra-ligand forces on the ortho-methyl substituent of 2 may cause the phenyl ring to twist out of the plane with respect to the pyridine, as observed in the crystal structure of BRDT-BDx/CDD-956 (compound 4) complex even in the absence of such a methyl/pyridine steric repulsion. The R enantiomer positions the methyl-amide DNA linker site such that it is oriented away from the protein surface, as observed in the crystal structure, which, without wishing to be bound by theory, may facilitate robust interaction in the DECL selection process. The mid-nanomolar affinities of compounds 3 and 5 (FIG. 3 and Table 2) suggest that the methyl-quinoline and phenyl-pyridine moieties of the S enantiomer may interact with BRDT-BD1 like the R enantiomer of compound 4 in the crystal structure provided herein; thereby orienting the methyl-amide of the S enantiomer towards W50 rather than towards solvent, incurring steric clashes for many of the possible rotamers of this substituent. Like the methyl of compound 2, the halides of compounds 8 and 9 can be accommodated without steric clashes.

TABLE 7
Data collection and refinement statistics^{a}

| Protein:ligand complex | BRDT-BD1:CDD-956 | |
|---|---|---|
| | (compound 4) | |
| Wavelength | 1.000 | |
| Resolution range | 48.37-1.82 | (1.885-1.82) |
| Space group | P 2₁ 2₁ 2₁ | |
| Unit cell | 61.69, 94.38, | |
| | 112.7 90 90 90 | |
| Total reflections | 759141 | (35916) |
| Unique reflections | 59693 | (5882) |
| Multiplicity | 12.7 | (6.1) |
| Completeness (%) | 99.98 | (100.00) |
| Mean I/sigma(I) | 8.30 | (0.72) |
| Wilson B-factor | 25.72 | |
| R-merge | 0.2041 | (2.094) |
| R-meas | 0.2122 | (2.288) |
| R-pim | 0.05729 | (0.9129) |
| CC1/2 | 0.991 | (0.306) |
| CC | 0.998 | (0.684) |
| Reflections used in refinement | 59690 | (5882) |
| Reflections used for R-free | 1909 | (189) |
| R-work | 0.1777 | (0.3321) |
| R-free | 0.1834 | (0.3363) |
| CC(work) | 0.967 | (0.641) |
| CC(free) | 0.971 | (0.548) |

| | |
|---|---|
| Number of non-hydrogen atoms | 4230 |
| macromolecules | 3581 |
| ligands | 178 |
| solvent | 477 |
| Protein residues | 434 |
| RMS(bonds) | 0.005 |
| RMS(angles) | 0.84 |
| Ramachandran favored (%) | 98.83 |
| Ramachandran allowed (%) | 0.94 |
| Ramachandran outliers (%) | 0.23 |
| Rotamer outliers (%) | 0.51 |
| Clash Score | 6.70 |
| Average B-factor | 33.91 |
| macromolecules | 33.15 |
| ligands | 29.71 |
| solvent | 41.15 |
| Number of TLS groups | 26 |

^{a}Statistics for the highest-resolution shell are shown in parentheses

### Example 9: BD1 Selectivity in Cells

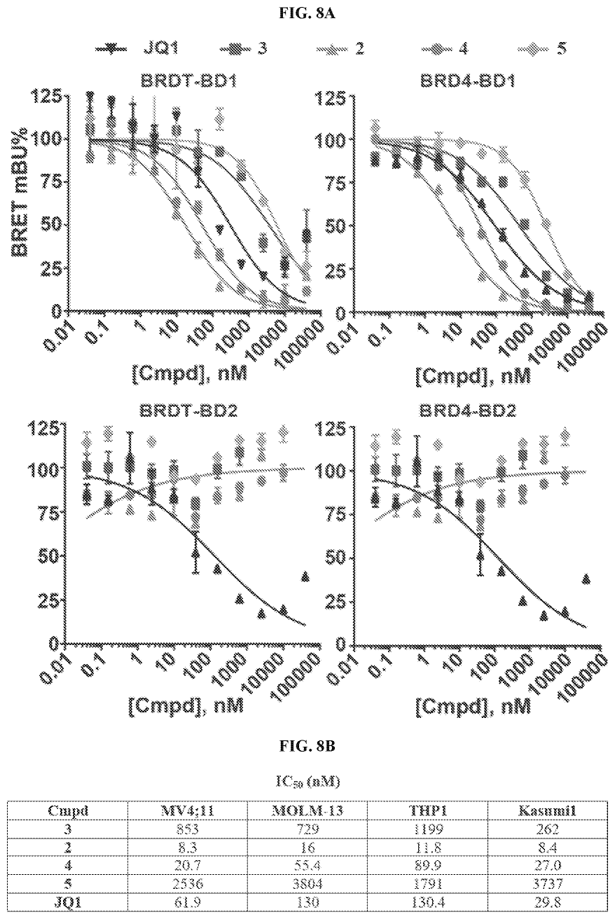

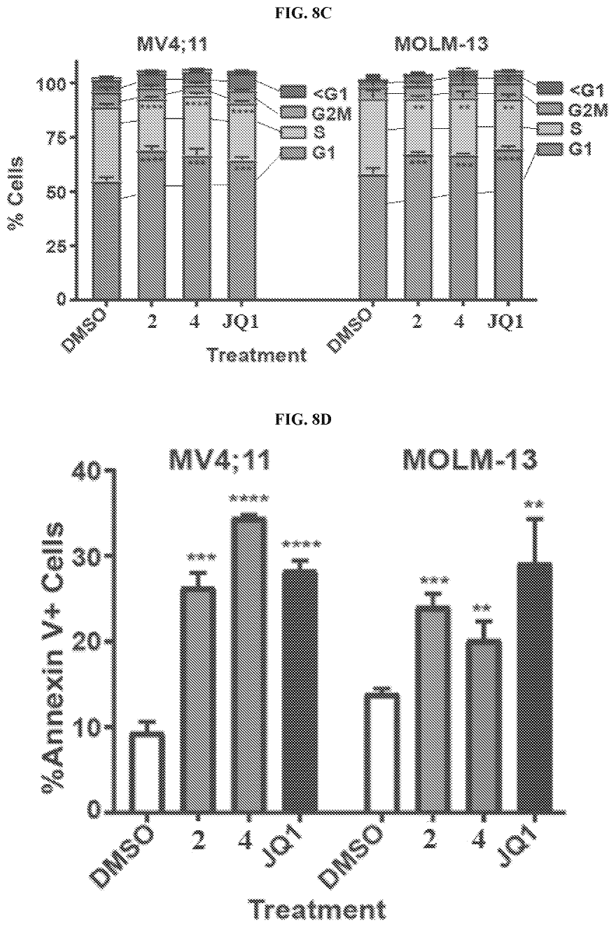

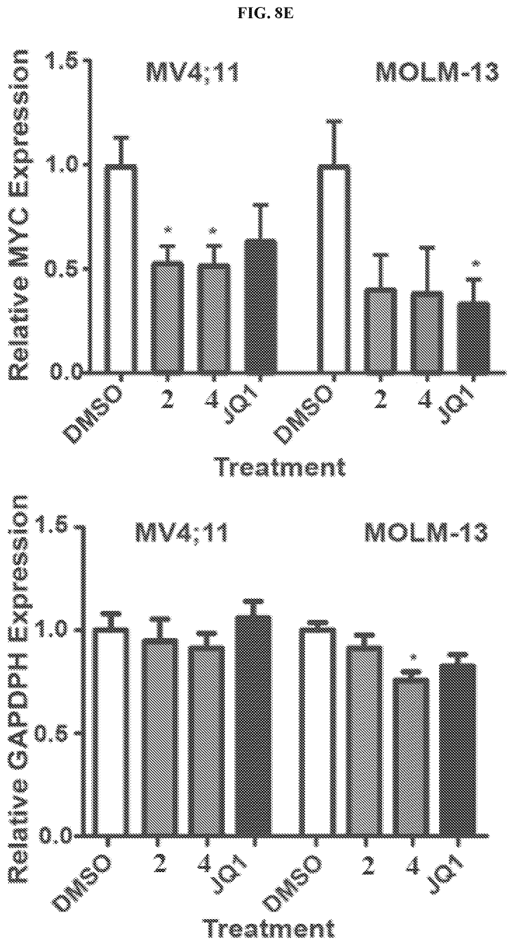

To confirm that the compounds were cell penetrant and active, the cellular binding of compounds 2-5 to BET bromodomains was evaluated using a NanoBRET target engagement assay that measures compound binding at select target proteins within intact cells. The inhibition of compounds 2 and 4 on tracer binding was found to be more potent and selective for NanoLuc-BET BD1 in transiently transfected HEK293 cells (FIG. 8A). There was no effect of compounds 2 and 4 against BRDT-BD2 or BRD4-BD2, compared to JQ1, which binds both BD1 and BD2 equally.

### Example 10: Activity in Acute Myeloid Leukemia (AML) Cell Lines

High levels of BRD4 are observed in multiple cancers, including leukenmias. To determine the effects of the BD1-selective compounds described herein, BRD4-dependent AML cell lines were treated with a range of doses of compounds to determine the IC₅₀ of each agent. A similar cellular potency as was detected in the NanoBRET assays was observed, with compound 3 having minimal effect, and compounds 2 and 4 having similar or slightly improved cellular potency than JQ1 (FIG. 8B). Compound 5 had minimal activity in the AML cell lines in analogous fashion to compound 3.

It was confirmed that both compounds 2 and 4 induced Gi arrest and apoptosis in both MV4; 11 and MOLM-13 cells (FIGS. 8C-8D). MYC expression was also suppressed after 8 hours of treatment with both agents in both cell lines, with no/minimal effects on GAPDH (FIG. 8E). These findings were compared to those of other SAR data and it was found that cellular potency and biochemical binding efficacy are correlated (Table 8).

TABLE 8
Viability EC₅₀ of selected compounds
against AML cell line MV4; 11

| Compound | MV4; 11 EC₅₀ (nM) |
|---|---|
| 1 | —^{a} |
| 2 | 8 |
| 4 | 21 |
| 7 | —^{a} |
| 8 | 58 |
| 9 | 100 |
| 10 | 143 |
| 13 | —^{a} |

^{a}not efficient.

### Enumerated Embodiments

The following exemplary embodiments are provided, the numbering of which is not to be construed as designating levels of importance:

Embodiment 1 provides a compound of formula (I), or a salt, solvate, prodrug, isotopologue, tautomer, or stereoisomer thereof:

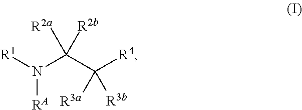

wherein:
   R¹ is

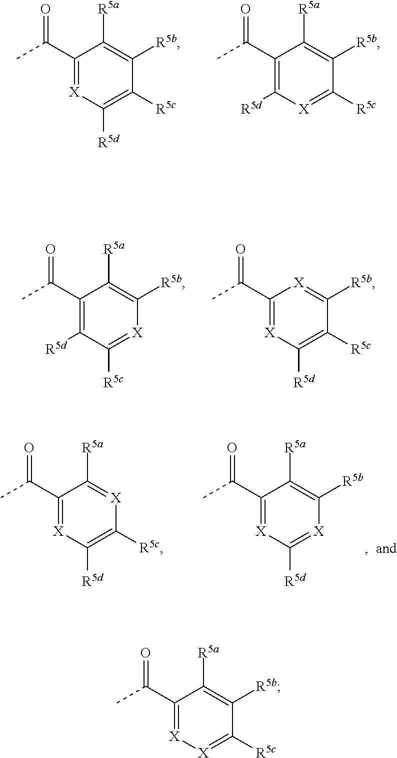

R^{2a} and R^{2b} are each independently selected from the group consisting of H, optionally substituted C₁-C₆ alkyl, and C(═O)N(R^{a})(R^{b}),
   wherein at least one of R^{2a} and R^{2b} is H;
R^{3a} and R^{3b} are each independently selected from the group consisting of H and C₁-C₆ alkyl;
R⁴ is

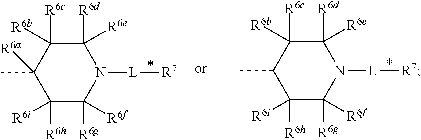

R^{5a}, R^{5b}, R^{5c}, R^{5d}, and R^{5e}, if present, are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂₀R^{a}, S(═O)₂R^{a}, S(═O)₂N(R^{a})(R^{b}), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
wherein two vicinal substituents selected from the group consisting of R^{5a}, R^{5b}, R^{5c}, R^{5d}, and R^{5e}, if present, may combine to form an optionally substituted phenyl or optionally substituted C₂-C₈ heteroaryl;
R^{6a}, R^{6b}, R^{6c}, R^{6d}, R^{6e}, R^{6f}, R^{6g}, R^{6h}, and R⁶ⁱ, if present, are each independently selected from the group consisting of H, halogen, optionally substituted C₁-C₆ alkyl, C₁-C₆ alkoxy, C₁-C₆ haloalkyl, C₁-C₆ haloalkoxy, optionally substituted C₃-C₈ cycloalkyl, and optionally substituted C₂-C₈ heterocyclyl;
R⁷ is

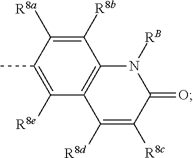

R^{8a}, R^{8b}, R^{8c}, R^{8d}, and R^{8e} are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂OR^{a}, S(═O)₂R^{a}, S(═O)₂N(R^{a})(R^{b}), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
X is selected from the group consisting of N and C(R^{5e});
L is selected from the group consisting of —C(═O)(optionally substituted C₁-C₃ alkylene)-*, —(C═O)—*, and -(optionally substituted C₁-C₃ alkylene)C(═O)—*,
   wherein * indicates the bond between L and R⁷;
R^{A} and R^{B} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl; and
R^{a} and R^{b} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl.

Embodiment 2 provides the compound of formula (I), which is selected from the group consisting of:

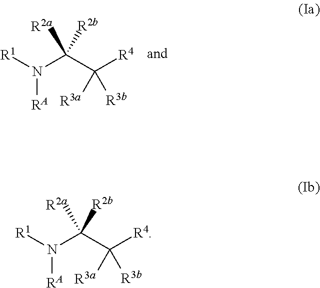

Embodiment 3 provides the compound of Embodiment 1 or 2, wherein X is N or CH.

Embodiment 4 provides the compound of any one of Embodiments 1-3, wherein R^{5a}, R^{5b}, R^{5c}, R^{5d}, and R^{5e}, if present, are each independently selected from the group consisting of H, CF₃, and phenyl optionally substituted with at least one substituent selected from the group consisting of C₁-C₆ alkyl and halogen.

Embodiment 5 provides the compound of Embodiment 4, wherein the phenyl in any one of R^{5a}, R^{5b}, R^{5c}, R^{5d}, and R^{5e} is selected from the group consisting of 2,4-dimethylphenyl, 4-chlorophenyl, and 3-fluoro-4-chlorophenyl.

Embodiment 6 provides the compound of any one of Embodiments 1-5, wherein R¹ is

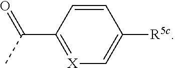

Embodiment 7 provides the compound of Embodiment 1 or 2, wherein R¹ is selected from the group consisting of

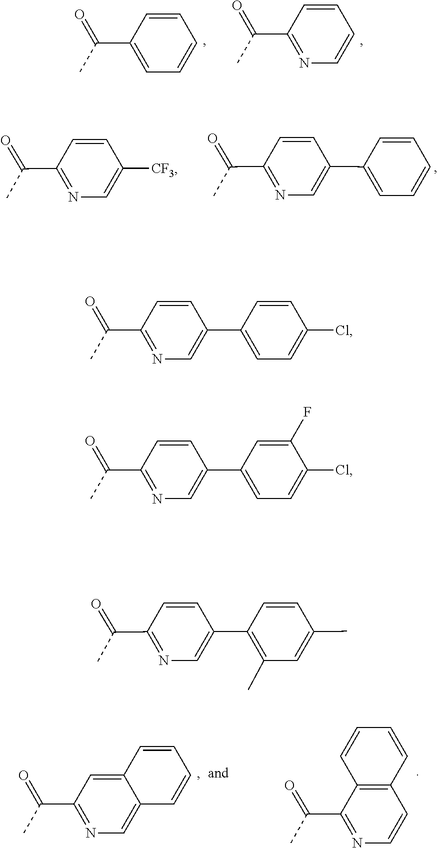

Embodiment 8 provides the compound of any one of Embodiments 1-7, wherein one of the following applies:
(a) R^{2a} is H and R^{2b} is H;
(b) R^{2a} is C(═O)NHMe and R^{2b} is H;
(c) R^{2a} is H and R^{2b} is C(═O)NHMe,
(d) R^{2a} is Me and R^{2b} is H; or
(e) R^{2a} is H and R^{2b} is Me.

Embodiment 9 provides the compound of any one of Embodiments 1-8, wherein at least one of the following applies:
(a) at least one of R^{6a}, R^{6b}, R^{6c}, R^{6d}, R^{5e}, R^{6f}, R_{6g}, R^{6h}, and R⁶ⁱ is H;
(b) at least two of R^{6a}, R^{6b}, R^{6c}, R^{6d}, R^{6e}, R^{6f}, R^{6g}, R^{6h}, and R⁶ⁱ are H;
(c) at least three of R^{6a}, R^{6b}, R^{6c}, R^{6d}, R^{6e}, R^{6f}, R^{6g}, R^{6h}, and R⁶ⁱ are H; (d) at least four of R^{6a}, R^{6b}, R^{6c}, R^{6d}, R^{6e}, R^{6f}, R^{6g}, R^{6h}, and R⁶ⁱ are H;
(e) at least five of R^{6a}, R^{6b}, R^{6c}, R^{6d}, R^{6e}, R^{6f}, R^{6g}, R^{6h}, and R⁶ⁱ are H;
(f) at least six of R^{6a}, R^{6b}, R^{6c}, R^{6d}, R^{6e}, R^{6f}, R^{6g}, R^{6h}, and R⁶ⁱ are H;
(g) at least seven of R^{6a}, R^{6b}, R^{6c}, R^{6d}, R^{6e}, R^{6f}, R^{6g}, R^{6h}, and R⁶ⁱ are H;
(h) at least eight of R^{6a}, R^{6b}, R^{6c}, R^{6d}, R^{6e}, R^{6f}, R^{6g}, R^{6h}, and R⁶ⁱ are H; and
(i) each of R^{6a}, R^{6b}, R^{6c}, R^{6d}, R^{6e}, R^{6f}, R^{6g}, R^{6h}, and R⁶ⁱ is H.

Embodiment 10 provides the compound of any one of Embodiments 1-9, wherein L is —C(═O)CH₂—*.

Embodiment 11 provides the compound of any one of Embodiments 1-10, wherein R^{8a}, R^{8b}, R^{8c}, R^{8d}, and R^{8e} are each independently selected from the group consisting of H and Me.

Embodiment 12 provides the compound of any one of Embodiments 1-11, wherein R⁷ is selected from the group consisting of:

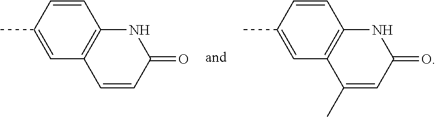

Embodiment 13 provides the compound of any one of Embodiments 1-12, wherein R⁴ is selected from the group consisting of:

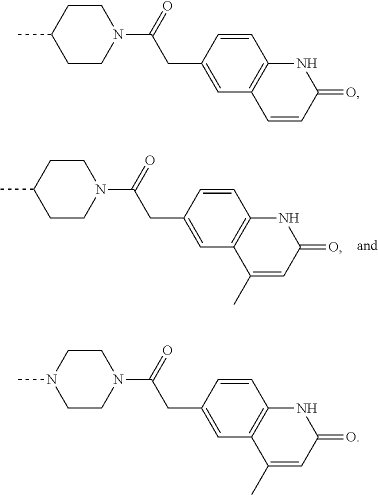

Embodiment 14 provides the compound of any one of Embodiments 1-13, wherein each occurrence of alkyl, haloalkyl, alkoxy, haloalkoxy, cycloalkyl, heterocyclyl, benzyl, phenyl, naphthyl, and heteroaryl is independently optionally substituted with at least one substituent selected from the group consisting of C₁-C₆ alkyl, C₃-C₈ cycloalkyl, C₃-C₆ allyl, C₃-C₆ propargyl, C₁-C₆ hydroxyalkyl, halogen, NO₂, CN, OH, NH₂, NH(C₁-C₆ alkyl), N(C₁-C₆ alkyl)₂, NH(C₆-C₁₀ aryl), N(C₆-C₁₀ aryl)₂, C₁-C₆ alkoxy, C₃-C₈ cycloalkoxy, C₁-C₃ haloalkyl, C₁-C₆ haloalkoxy, C₃-C₈ halocycloalkoxy, benzyl, phenyl, naphthyl, C₂-C₈ heterocyclyl, C(═O)H, C(═O)(C₁-C₆ alkyl), C(═O)(C₆-C₁₀ aryl), C(═O)O(benzyl), C(═O)(C₃-C₈ cycloalkyl), C(═O)OH, C(═O)O(C₁-C₆ alkyl), C(═O)O(C₆-C₁₀ aryl), OC(═O)H, OC(═O)(C₁-C₆ alkyl), OC(═O)(C₆-C₁₀ aryl), OC(═O)OH, OC(═O)O(C-C₆ alkyl), OC(═O)O(C₆-C₁₀ aryl), SH, S(C₁-C₆ alkyl), S(C₆-C₁₀ aryl), S(═O)(C₁-C₆ alkyl), S(═O)(C₆-C₁₀ aryl), S(═O)₂₀H, S(═O)₂O(C₁-C₆ alkyl), S(═O)₂O(C₆-C₁₀ aryl), S(═O)₂(C₁-C₆ alkyl), S(═O)₂(C₆-C₁₀ aryl), S(═O)₂NH₂, S(═O)₂NH(C₁-C₆ alkyl), S(═O)₂N(C₁-C₆ alkyl)₂, S(═O)₂NH(C₆-C₁₀ aryl), S(═O)₂N(C₆-C₁₀ aryl)₂, S(═O)₂NHC(═O)NH₂, S(═O)₂N(C₁-C₆ alkyl)C(═O)NH₂, S(═O)₂NHC(═O)NH(C₁-C₆ alkyl), S(═O)₂N(C₁-C₆ alkyl)C(═O)NH(C₁-C₆ alkyl), S(═O)₂NHC(═O)NH(C₆-C₁₀ aryl), NHS(═O)₂(C₁-C₆ alkyl), N(C₁-C₆ alkyl)S(═O)₂(C₁-C₆ alkyl), NHS(═O)₂(C₆-C₁₀ aryl), N(C₁-C₆ alkyl)S(═O)₂(C₆-C₁₀ aryl), NHC(═O)H, NHC(═O)(C₁-C₆ alkyl), N(C₁-C₆ alkyl)C(═O)(C₁-C₆ alkyl), NHC(═O)(C₆-C₁₀ aryl), N(C₁-C₆ alkyl)C(═O)(C₆-C₁₀ aryl), C(═O)NH₂, C(═O)NH(C₁-C₆ alkyl), C(═O)N(C₁-C₆ alkyl)₂, C(═O)NH(C₆-C₁₀ aryl), C(═O)N(C₁-C₆ alkyl)(C₆-C₁₀ aryl), and C(═O)N(C₆-C₁₀ aryl)₂.

Embodiment 15 provides the compound of Embodiment 14, wherein each optional substituent in the alkyl, haloalkyl, alkoxy, haloalkoxy, cycloalkyl, heterocyclyl, benzyl, phenyl, naphthyl, and heteroaryl is further optionally substituted with at least one substituent selected from the group consisting of C₁-C₆ alkyl, C₁-C₃ haloalkyl, C₁-C₃ haloalkoxy, C₁-C₆ heteroalkyl, halogen, CN, NO₂, OH, O(C₁-C₆ alkyl), NH₂, NH(C₁-C₆ alkyl), N(C₁-C₆ alkyl)₂, C(═O)OH, C(═O)O(C₁-C₆ alkyl), C(═O)NH₂, C(═O)NH(C₁-C₆ alkyl), C(═O)N(C₁-C₆ alkyl)₂, NH(C═NH)NH₂, and imidazolyl.

Embodiment 16 provides the compound of any one of Embodiments 1-15, which is selected from the group consisting of.
5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(R)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(S)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
N-(1-(methylamino)-1-oxo-3-(1-(2-(2-oxo-1,2-dihydroquinolin-6-yl)acetyl)piperidin-4-yl)propan-2-yl)-5-phenylpicolinamide;
(R)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
(S)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(R)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(S)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
5-(2,4-dimethylphenyl)-N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)benzamide;
N-(2-(1-(2-(4-methyl-2-oxo-1,2-dihydroquinolin-6-yl)acetyl)piperidin-4-yl)ethyl)quinoline-2-carboxamide;
N-(2-(1-(2-(4-methyl-2-oxo-1,2-dihydroquinolin-6-yl)acetyl)piperidin-4-yl)ethyl)isoquinoline-3-carboxamide;
N-(2-(1-(2-(4-methyl-2-oxo-1,2-dihydroquinolin-6-yl)acetyl)piperidin-4-yl)ethyl)isoquinoline-1-carboxamide; and
N-(2-(4-(2-(4-methyl-2-oxo-1,2-dihydroquinolin-6-yl)acetyl)piperazin-1-yl)ethyl)picolinamide

Embodiment 17 provides a pharmaceutical composition comprising the compound of any one of Embodiments 1-16 and a pharmaceutically acceptable carrier.

Embodiment 18 provides a method of inhibiting bromodomain testis (BRDT) in a male subject, the method comprising administering to the male subject a therapeutically effective amount of at least one compound of any one of Embodiments 1-16 and/or the pharmaceutical composition of Embodiment 17.

Embodiment 19 provides a method of promoting male contraception and/or infertility in a male subject, the method comprising administering to the male subject a therapeutically effective amount of at least one compound of any one of Embodiments 1-16 and/or the pharmaceutical composition of Embodiment 17.

Embodiment 20 provides a method of minimizing and/or reducing spermatozoa number and/or motility in a male subject, the method comprising administering to the male subject a therapeutically effective amount of at least one compound of any one of Embodiments 1-16 and/or the pharmaceutical composition of Embodiment 17.

Embodiment 21 provides the method of any one of Embodiments 18-20, wherein the compound provides a contraceptive effect in the male.

Embodiment 22 provides a method of treating, preventing, and/or ameliorating cancer, an inflammatory condition, and/or a metabolic disorder in a subject, the method comprising administering to the subject a therapeutically effective amount of at least one compound of any one of Embodiments 1-16 and/or the pharmaceutical composition of Embodiment 17.

Embodiment 23 provides a method of treating, preventing, and/or ameliorating an infectious disease in a subject, the method comprising administering to the subject a therapeutically effective amount of at least one compound of any one of Embodiments 1-16 and/or the pharmaceutical composition of Embodiment 17.

Embodiment 24 provides the method of Embodiment 23, wherein the infectious disease is a viral infection.

Embodiment 25 provides the method of Embodiment 24, wherein the viral infection is caused by a coronavirus.

Embodiment 26 provides the method of Embodiment 25, wherein the coronavirus is SARS-CoV-2.

Embodiment 27 provides the method of any one of Embodiments 18-26, wherein the compound inhibits BRDT bromodomain-1 (BRDT-BD1).

Embodiment 28 provides the method of any one of Embodiments 18-27, wherein the compound selectively inhibits BRDT-BD1 over BRDT bromodomain-2 (BRDT-BD2).

Embodiment 29 provides the method of any one of Embodiments 18-28, wherein the compound inhibits BRD4 bromodomain-1 (BRD4-BD1).

Embodiment 30 provides the method of any one of Embodiments 18-29, wherein the compound selectively inhibits BRD4-BD1 over BRD4 bromodomain-2 (BRD4-BD2).

Embodiment 31 provides the method of any one of Embodiments 18-30, wherein the compound inhibits BRD3 bromodomain-1 (BRD3-BD1).

Embodiment 32 provides the method of any one of Embodiments 18-31, wherein the compound selectively inhibits BRD3-BD1 over BRD3 bromodomain-2 (BRD3-BD2).

Embodiment 33 provides the method of any one of Embodiments 18-32, wherein the compound inhibits BRD2 bromodomain-1 (BRD2-BD1).

Embodiment 34 provides the method of any one of Embodiments 18-33, wherein the compound selectively inhibits BRD2-BD1 over BRD2 bromodomain-2 (BRD2-BD2).

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I), or a salt, solvate, prodrug, isotopologue, tautomer, or stereoisomer thereof: 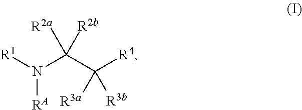
wherein:
R¹ is 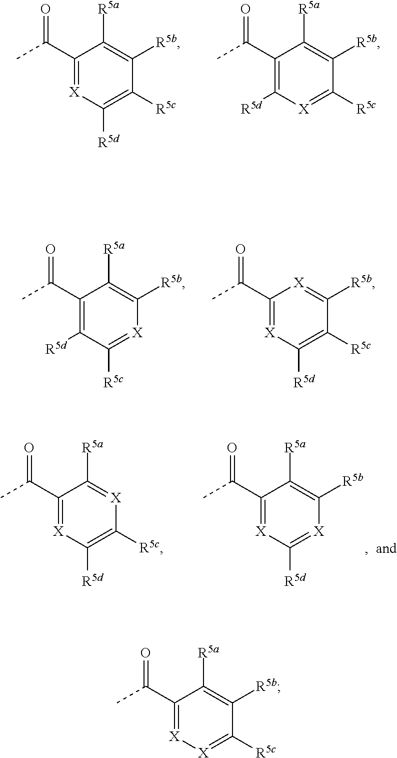
R^{2a} and R^{2b} are each independently selected from the group consisting of H, optionally substituted C₁-C₆ alkyl, and C(═O)N(R^{a})(R),
wherein at least one of R^{2a} and R^{2b} is H;
R^{3a} and R^{3b} are each independently selected from the group consisting of H and C₁-C₆ alkyl;
R⁴ is 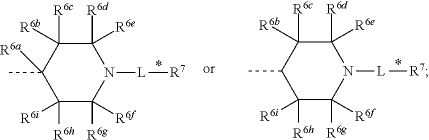
R^{5a}, R^{5b}, R^{5c}, R^{5d}, and R^{5e}, if present, are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂OR^{a}, S(═O)₂R^{a}, S(═O)₂N(R^{a})(R), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
wherein two vicinal substituents selected from the group consisting of R^{5a}, R^{5b}, R^{5c}, R^{5d}, and R^{5e}, if present, may combine to form an optionally substituted phenyl or optionally substituted C₂-C₈ heteroaryl;
R^{6a}, R^{6b}, R^{6c}, R^{6d}, R^{6e}, R^{6f}, R^{6g}, R^{6h}, and R⁶ⁱ, if present, are each independently selected from the group consisting of H, halogen, optionally substituted C₁-C₆ alkyl, C₁-C₆ alkoxy, C₁-C₆ haloalkyl, C₁-C₆ haloalkoxy, optionally substituted C₃-C₈ cycloalkyl, and optionally substituted C₂-C₈ heterocyclyl;
R⁷ is 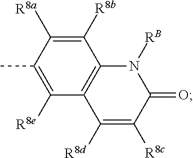
R^{8a}, R^{8b}, R^{8c}, R^{8d}, and R^{8e} are each independently selected from the group consisting of H, halogen, CN, NO₂, OR^{a}, N(R^{a})(R^{b}), C(═O)R^{a}, C(═O)OR^{a}, C(═O)N(R^{a})(R^{b}), N(R^{a})C(═O)R^{a}, OC(═O)R^{a}, C(═O)H, S(═O)R^{a}, S(═O)₂OR^{a}, S(═O)₂R^{a}, S(═O)₂N(R^{a})(R^{b}), optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, optionally substituted naphthyl, and optionally substituted C₂-C₈ heterocyclyl;
X is selected from the group consisting of N and C(R^{5e});
L is selected from the group consisting of —C(═O)(optionally substituted C₁-C₃ alkylene)-*, —(C═O)—*, and -(optionally substituted C₁-C₃ alkylene)C(═O)—*,
wherein * indicates the bond between L and R⁷;
R^{A} and R^{B} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl; and
R^{a} and R^{b} are each independently selected from the group consisting of H, optionally substituted C₁-C₆ alkyl, C₃-C₈ cycloalkyl, C₁-C₆ haloalkyl, C₂-C₈ heterocyclyl, optionally substituted benzyl, and optionally substituted phenyl.

2. The compound of claim 1, which is selected from the group consisting of:

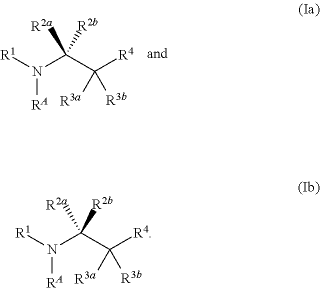

3. The compound of claim 1, wherein X is N or CH.

4. The compound of claim 1, wherein R^{5a}, R^{5b}, R^{5c}, R^{5d}, and R^{5f}, if present, are each independently selected from the group consisting of H, CF₃, and phenyl optionally substituted with at least one substituent selected from the group consisting of C₁-C₆ alkyl and halogen, optionally wherein the phenyl in any one of R^{5a}, R^{5b}, R^{5c}, R^{5d}, and R^{5e} is selected from the group consisting of 2,4-dimethylphenyl, 4-chlorophenyl, and 3-fluoro-4-chlorophenyl.

5. (canceled)

6. The compound of claim 1, wherein R¹ is

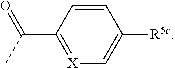

7. The compound of claim 1, wherein R¹ is selected from the group consisting of:

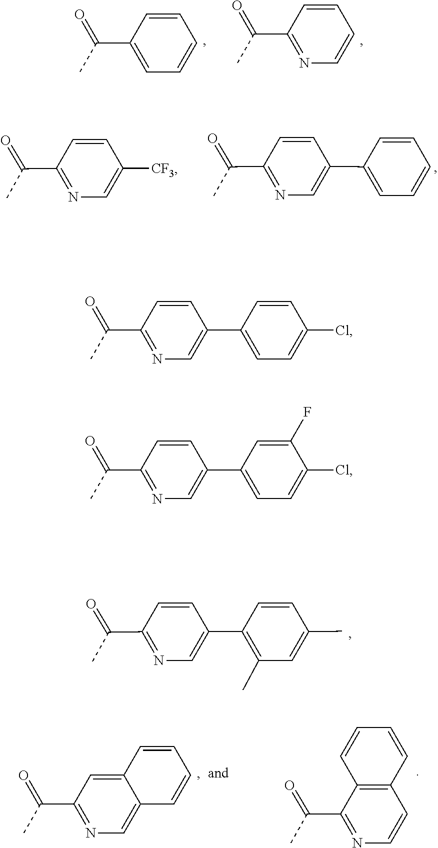

8. The compound of claim 1, wherein one of the following applies:
(a) R^{2a} is H and R^{2b} is H;
(b) R^{2a} is C(═O)NHMe and R^{2b} is H;
(c) R^{2a} is H and R^{2b} is C(═O)NHMe;
(d) R^{2a} is Me and R^{2b} is H; or
(e) R^{2a} is H and R^{2b} is Me.

9. The compound of claim 1, wherein at least one of the following applies:
(a) at least one of R^{6a}, R^{6b}, R^{6c}, R^{6d}, R^{6e}, R^{6f}, R^{6g}, R^{6h}, and R⁶ⁱ is H;
(b) at least two of R^{6a}, R^{6b}, R^{6c}, R^{6d}, R^{6e}, R^{6f}, R^{6g}, R^{6h}, and R⁶ⁱ are H;
(c) at least three of R^{6a}, R^{6b}, R^{6c}, R^{6d}, R^{6e}, R^{6f}, R^{6g}, R^{6h}, and R⁶ⁱ are H;
(d) at least four of R^{6a}, R^{6b}, R^{6c}, R^{6d}, R^{6e}, R^{6f}, R^{6g}, R^{6h}, and R⁶ⁱ are H;
(e) at least five of R^{6a}, R^{6b}, R^{6c}, R^{6d}, R^{6e}, R^{6f}, R^{6g}, R^{6h}, and R⁶ⁱ are H;
(f) at least six of R^{6a}, R^{6b}, R^{6c}, R^{6d}, R^{6e}, R^{6f}, R^{6g}, R^{6h}, and R⁶ⁱ are H;
(g) at least seven of R₆ₐ, R^{6b}, R^{6c}, R^{6d}, R^{6e}, R^{6f}, R^{6g}, R^{6h}, and R⁶ⁱ are H;
(h) at least eight of R^{6a}, R^{6b}, R^{6c}, R^{6d}, R^{6e}, R^{6f}, R^{6g}, R^{6h}, and R⁶ⁱ are H; and
(i) each of R^{6a}, R^{6b}, R^{6c}, R^{6d}, R^{6e}, R^{6f}, R^{6g}g, R^{6h}, and R⁶ⁱ is H.

10. The compound of claim 1, wherein L is —C(═O)CH₂—*.

11. The compound of claim 1, wherein R^{8a}, R^{8b}, R^{8c}, R^{8d}, and R^{8e} are each independently selected from the group consisting of H and Me.

12. The compound of claim 1, wherein R⁷ is selected from the group consisting of:

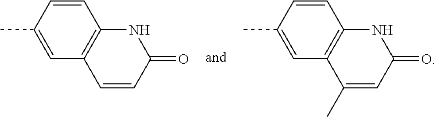

13. The compound of claim 1, wherein R⁴ is selected from the group consisting of:

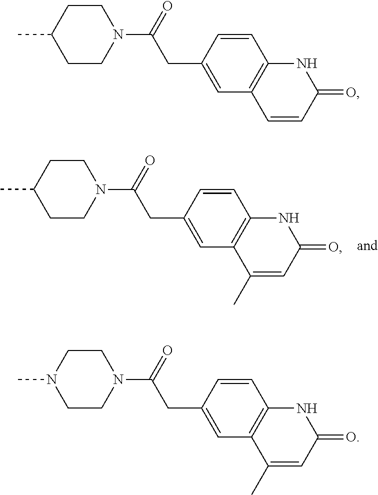

14. The compound of claim 1, wherein each occurrence of alkyl, haloalkyl, alkoxy, haloalkoxy, cycloalkyl, heterocyclyl, benzyl, phenyl, naphthyl, and heteroaryl is independently optionally substituted with at least one substituent selected from the group consisting of C₁-C₆ alkyl, C₃-C₈ cycloalkyl, C₃-C₆ allyl, C₃-C₆ propargyl, C₁-C₆ hydroxyalkyl, halogen, NO₂, CN, OH, NH₂, NH(C₁-C₆ alkyl), N(C₁-C₆ alkyl)₂, NH(C₆-C₁₀ aryl), N(C₆-C₁₀ aryl)₂, C₁-C₆ alkoxy, C₃-C₈ cycloalkoxy, C₁-C₃ haloalkyl, C₁-C₆ haloalkoxy, C₃-C₈ halocycloalkoxy, benzyl, phenyl, naphthyl, C₂-C₈ heterocyclyl, C(═O)H, C(═O)(C₁-C₆ alkyl), C(═O)(C₆-C₁₀ aryl), C(═O)O(benzyl), C(═O)(C₃-C₈ cycloalkyl), C(═O)OH, C(═O)O(C₁-C₆ alkyl), C(═O)O(C₆-C₁₀ aryl), OC(═O)H, OC(═O)(C₁-C₆ alkyl), OC(═O)(C₆-C₁₀ aryl), OC(═O)OH, OC(═O)O(C₁-C₆ alkyl), OC(═O)O(C₆-C₁₀ aryl), SH, S(C₁-C₆ alkyl), S(C₆-C₁₀ aryl), S(═O)(C₁-C₆ alkyl), S(═O)(C₆-C₁₀ aryl), S(═O)₂₀H, S(═O)₂O(C₁-C₆ alkyl), S(═O)₂O(C₆-C₁₀ aryl), S(═O)₂(C₁-C₆ alkyl), S(═O)₂(C₆-C₁₀ aryl), S(═O)₂NH₂, S(═O)₂NH(C₁-C₆ alkyl), S(═O)₂N(C₁-C₆ alkyl)₂, S(═O)₂NH(C₆-C₁₀ aryl), S(═O)₂N(C₆-C₁₀ aryl)₂, S(═O)₂NHC(═O)NH₂, S(═O)₂N(C₁-C₆ alkyl)C(═O)NH₂, S(═O)₂NHC(═O)NH(C₁-C₆ alkyl), S(═O)₂N(C₁-C₆ alkyl)C(═O)NH(C₁-C₆ alkyl), S(═O)₂NHC(═O)NH(C₆-C₁₀ aryl), NHS(═O)₂(C₁-C₆ alkyl), N(C₁-C₆ alkyl)S(═O)₂(C₁-C₆ alkyl), NHS(═O)₂(C₆-C₁₀ aryl), N(C₁-C₆ alkyl)S(═O)₂(C₆-C₁₀ aryl), NHC(═O)H, NHC(═O)(C₁-C₆ alkyl), N(C₁-C₆ alkyl)C(═O)(C₁-C₆ alkyl), NHC(═O)(C₆-C₁₀ aryl), N(C₁-C₆ alkyl)C(═O)(C₆-C₁₀ aryl), C(═O)NH₂, C(═O)NH(C₁-C₆ alkyl), C(═O)N(C₁-C₆ alkyl)₂, C(═O)NH(C₆-C₁₀ aryl), C(═O)N(C₁-C₆ alkyl)(C₆-C₁₀ aryl), and C(═O)N(C₆-C₁₀ aryl)₂.

15. The compound of claim 14, wherein each optional substituent in the alkyl, haloalkyl, alkoxy, haloalkoxy, cycloalkyl, heterocyclyl, benzyl, phenyl, naphthyl, and heteroaryl is further optionally substituted with at least one substituent selected from the group consisting of C₁-C₆ alkyl, C₁-C₃ haloalkyl, C₁-C₃ haloalkoxy, C₁-C₆ heteroalkyl, halogen, CN, NO₂, OH, O(C₁-C₆ alkyl), NH₂, NH(C₁-C₆ alkyl), N(C₁-C₆ alkyl)₂, C(═O)OH, C(═O)O(C₁-C₆ alkyl), C(═O)NH₂, C(═O)NH(C₁-C₆ alkyl), C(═O)N(C₁-C₆ alkyl)₂, NH(C═NH)NH₂, and imidazolyl.

16. The compound of claim 1, which is selected from the group consisting of:
5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(R)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
(S)-5-(2,4-dimethylphenyl)-N-(3-(1-(2-(4-methyl-2-oxo-1,2-dihydroquinolin-6-yl) acetyl) piperidin-4-yl)-1-(methylamino)-1-oxopropan-2-yl)picolinamide;
N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)-5-(trifluoromethyl)picolinamide;
5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chlorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-5-(4-chloro-3-fluorophenyl)-N-(3-(1-(2-(4-methyl-2-oxo-1,2-dihydroquinolin-6-yl)acetyl)piperidin-4-yl)-1-(methylamino)-1-oxopropan-2-yl)picolinamide;
N-(1-(methylamino)-1-oxo-3-(1-(2-(2-oxo-1,2-dihydroquinolin-6-yl)acetyl)piperidin-4-yl)propan-2-yl)-5-phenylpicolinamide;
(R)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
(S)-N-(1-(methylamino)-1-oxo-3-(1-(2-(2-oxo-1,2-dihydroquinolin-6-yl)acetyl)piperidin-4-yl)propan-2-yl)-5-phenylpicolinamide;
N-(3-(1-(2-(4-methyl-2-oxo-1,2-dihydroquinolin-6-yl)acetyl)piperidin-4-yl)-1-(methylamino)-1-oxopropan-2-yl)picolinamide;
(R)-N-(3-(1-(2-(4-methyl-2-oxo-1,2-dihydroquinolin-6-yl)acetyl)piperidin-4-yl)-1-(methylamino)-1-oxopropan-2-yl)picolinamide;
(S)-N-(3-(1-(2-(4-methyl-2-oxo-1,2-dihydroquinolin-6-yl)acetyl)piperidin-4-yl)-1-(methylamino)-1-oxopropan-2-yl)picolinamide;
5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(R)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
(S)-5-(2,4-dimethylphenyl)-N-(1-(1-(2-(4-methyl-2-oxo-1,2-dihydroquinolin-6-yl)acetyl)piperidin-4-yl)propan-2-yl)picolinamide;
5-(2,4-dimethylphenyl)-N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)picolinamide;
N-(2-(1-(2-(4-methyl-2-oxo-1,2-dihydroquinolin-6-yl)acetyl)piperidin-4-yl)ethyl)benzamide;
N-(2-(1-(2-(4-methyl-2-oxo-1,2-dihydroquinolin-6-yl)acetyl)piperidin-4-yl)ethyl)quinoline-2-carboxamide;
N-(2-(1-(2-(4-methyl-2-oxo-1,2-dihydroquinolin-6-yl)acetyl)piperidin-4-yl)ethyl)isoquinoline-3-carboxamide;
N-(2-(1-(2-(4-methyl-2-oxo-1,2-dihydroquinolin-6-yl)acetyl)piperidin-4-yl)ethyl)isoquinoline-1-carboxamide; and
N-(2-(4-(2-(4-methyl-2-oxo-1,2-dihydroquinolin-6-yl)acetyl)piperazin-1-yl)ethyl)picolinamide,
or a salt, solvate, prodrug, isotopologue, tautomer, or stereoisomer thereof.

17. A pharmaceutical composition comprising the compound of claim 1 and a pharmaceutically acceptable carrier.

18. A method of inhibiting bromodomain testis (BRDT) in a male subject, the method comprising administering to the male subject a therapeutically effective amount of at least one compound of claim 1, optionally wherein the compound provides a contraceptive effect in the male.

19. A method of promoting male contraception or infertility in a male subject, the method comprising administering to the male subject a therapeutically effective amount of at least one compound of claim 1.

20. A method of minimizing or reducing spermatozoa number or motility in a male subject, the method comprising administering to the male subject a therapeutically effective amount of at least one compound of claim 1, optionally wherein the compound provides a contraceptive effect in the male.

21. (canceled)

22. A method of treating, preventing, or ameliorating cancer, an inflammatory condition, or a metabolic disorder in a subject, the method comprising administering to the subject a therapeutically effective amount of at least one compound of claim 1.

23. A method of treating, preventing, or ameliorating an infectious disease in a subject, the method comprising administering to the subject a therapeutically effective amount of at least one compound of claim 1.

24. The method of claim 23, wherein the infectious disease is a viral infection, optionally wherein the viral infection is caused by a coronavirus, preferably wherein the coronavirus is SARS-CoV-2.

25. (canceled)

26. (canceled)

27. The method of claim 18, wherein at least one of the following applies:
(a) the compound inhibits BRDT bromodomain-1 (BRDT-BD1);
(b) the compound selectively inhibits BRDT-BD1 over BRDT bromodomain-2 (BRDT-BD2);
(c) the compound inhibits BRD4 bromodomain-1 (BRD4-BD1);
(d) the compound selectively inhibits BRD4-BD1 over BRD4 bromodomain-2 (BRD4-BD2);
(e) the compound inhibits BRD3 bromodomain-1 (BRD3-BD1);
(f) the compound selectively inhibits BRD3-BD1 over BRD3 bromodomain-2 (BRD3-BD2);
(g) the compound inhibits BRD2 bromodomain-1 (BRD2-BD1);
(h) the compound selectively inhibits BRD2-BD1 over BRD2 bromodomain-2 (BRD2-BD2).

28-34. (canceled)
